(19) United States
(12) Patent Application Publication
(10) Pub. No.: US 2010/0330120 A1
(43) Pub. Date: Dec. 30, 2010

(54) HUMAN PARAINFLUENZA VIRUSES HAVING SEPARATED P AND C GENES

(76) Inventors: Peter L. Collins, Silver Spring, MD (US); Brian R. Murphy, Bethesda, MD (US); Mario H. Skiadopoulos, Potomac, MD (US); Emmalene J. Bartlett, Chevy Chase, MD (US); Alexander C. Schmidt, Bethesda, MD (US); Ann-Marie M. Cruz, Bethesda, MD (US)

Correspondence Address: OTT- NIH;c/o WOODCOCK WASHBURN LLP, CIRA CENTRE, 12TH FLOOR, 2929 ARCH STREET, PHILADELPHIA, PA, 19104-2891, US

(21) Appl. No.: 12/599,095

(22) PCT Filed: May 8, 2008
(86) PCT No.: PCT/US08/63033

§ 371 Date: Sep. 16, 2010

Related U.S. Application Data
(60) Provisional application No. 60/916,799, filed on May 8, 2007.

(51) Int. Cl. A61K 39/155; C12N 7/01; C07H 21/04; C12N 7/04; A61P 37/04; A61P 31/14

(52) U.S. Cl. 424/211.1; 435/235.1; 536/23.72; 435/236

(57) ABSTRACT

The invention provides self replicating infectious recombinant paramyxoviruses where the P and C genes are separated rated. The recombinant paramyxoviruses preferably have one or more attenuating mutations and/or at least one temperature sensitive mutation and one non-temperature sensitive mutation. In some embodiments, the recombinant paramyxovirus has a separate variant polynucleotide encoding a C protein and a separate monocistronic polynucleotide encoding a P protein. Also provided are compositions and methods for using the recombinant paramyxoviruses as described herein.

### BACKGROUND OF THE INVENTION

Human parainfluenza viruses (HPIVs) are members of the Paramyxovirinae subfamily of the Paramyxoviridae family of viruses. Paramyxoviruses are enveloped viruses that replicate in the cytoplasm and bud at the plasma membrane and have a single-stranded negative-sense non-segmented RNA genome of approximately 13-19 kb. HPIVs are important pathogens in human populations, causing severe lower respiratory tract infections in infants and young children. Human parainfluenza virus type 1 (HPIV1) and type 2 (HPIV2) are the principal etiologic agents of laryngotracheobronchitis (croup) and also cause pneumonia and bronchitis (Chanock et al., 2001, Parainfluenza Viruses 4th Ed., Knipe et al. eds., Lippincott (Philadelphia, Pa.) 1341-1379). Human parainfluenza virus type 3 (HPIV3) is a leading cause of hospitalization for viral lower respiratory tract disease in infants and young children (Chanock et al., 2001, supra). HPIVs are also important causes of respiratory tract disease in adults. Collectively, HPIV1, HPIV2, and HPIV3 have been identified as the etiologic agents responsible for approximately 18% of hospitalizations for pediatric respiratory disease (Murphy et al., 1988, Virus Res., 11:1-15). HPIVs have also been implicated in a significant proportion of cases of virally induced middle ear effusions in children with otitis media (Heikkinen et al., 1999, N. Engl. J. Med., 340:260-264).

Despite considerable efforts, there are currently no parainfluenza virus vaccines available. Attenuated paramyxoviruses suitable for use in vaccines are currently under development. Two live attenuated HPIV3 vaccine candidates, a temperature sensitive (ts) derivative of the wild type HPIV3 JS strain and a bovine PIV3 strain, are currently being tested. (Karron et al, Pediatric Infectious Dis. J., 15:650, 1996; Karron et al, J. Infect. Dis., 171:1107, 1995; Karron et al., J. Infect. Dis., 172:1445, 1995). A chimeric PIV1 vaccine candidate has been generated by replacing the PIV3 HN and F open reading frames with those of PIV1 in a PIV3 full length cDNA (Tao et al., 2000a). A chimeric HPIV3 bearing the glycoproteins of HPIV2 was also generated previously (Tao et al., 2000b). Attenuated HPIV2 strains have previously been made by introducing mutations into the L protein (WO 04/027037). Recombinant viruses include HPIV3 recombinant viruses having three identified mutations in the L gene. (Skiadopoulos et al, J. Virol. 72:1762, 1998; Tao et al, J. Virol. 72:2955, 1998; Tao et al, Vaccine, 17:1100, 1999). These live attenuated vaccine candidates can induce protection against HPIV3 infection in some experimental animal models. (Karron et al, J. Infect. Dis., 172:1445, 1995b; Skiadopoulos et al, Vaccine 18:503, 1999; Skiadopoulos, Virology, 297: 136, 2002). However immunity to previous HPIV3 infection could limit the use of chimeric HPIV3 vaccines bearing heterologous HPIV1 or HPIV2 glycoproteins. Strategies to develop live viral vaccines are important in the design of safe and stable viral vaccine candidates.

In addition to providing possible vaccine candidates for protection against parainfluenza virus infection and disease, candidate vaccines may also be useful in expressing heterologous viral antigens. Studies demonstrate that foreign genes may be inserted into a paramyxovirus genome and are well expressed. (Bukreyev et al. J. Virol. 80:10293, 2006; Bukereyev et al, J. Virol., 70:6634, 1996; Hassan et al, Virology, 237:249, 1997; Jin et al, Virology 251:206, 1998; Schmidt et al., 2001; Skiadopoulos et al., 2002). However, in order to develop vectors for vaccine use, the level of protein expression is not the only influencing factor. Factors to be considered in the design of a vector for delivery of heterologous antigens include viral host range, immunogenicity, and pathogenicity. Some negative strand viruses are undesirable as vectors because of their pathogenicity, such as measles and rabies virus.

Thus, there remains a need to develop effective immunogenic compositions to alleviate health problems associated with HPIVs and other pathogens, and to immunize against multiple HPIV serotypes. There is also a need to develop immunogenic compositions to deliver heterologous antigens.

Each HPIV1 gene encodes a single protein, with the exception of the P/C gene, which contains two open reading frames (ORFs), one coding for the essential P protein and one coding for the non-essential C proteins, i.e., four related accessory proteins referred to as C′, C, Y1 and Y2. The P/C genes of HPIV3 are similarly organized, although the P gene of HPIV3 further encodes a D protein accessed by RNA editing and an internal ORF encoding a V protein. (See, e.g. WO 98/53078.) The C ORFs of SeV and HPIV1 are accessed by more than one translational start site to give rise to a nested set of carboxy-coterminal proteins called C′, C, Y1 and Y2, listed in the order of appearance of their respective translational start sites. Expression of the Y2 protein, whose existence is predicted based on the nucleotide sequence and by analogy to Sendai virus (SeV, murine PIV1), has not been directly confirmed for HPIV1. C proteins are expressed by viruses of the Respirovirus, Morbillivirus and Henipahvirus genera, but not by viruses of the Rubulavirus and Avulavirus genera. The C proteins are multifunctional, based mainly on studies with SeV. Their most prominent roles are (i) inhibition of the induction of interferon (IFN) a/11 by the host cell in response to viral infection and (ii) inhibition of intracellular signaling mediated by IFN α/β and γ. Other functions of C include down-regulating viral RNA synthesis, facilitating virion budding and release, and inhibiting cellular apoptosis (Karron and Collins, Chapter 42 in Field's Virology, 5^{th} ed., 2007).

The P protein also has several functions. It interacts with the N and L proteins and is essential in forming the active RNA-dependant RNA polymerase (N, P, L). The P protein acts as a chaperone and it might play a role in modulation of the host's immune response.

### DESCRIPTION OF THE INVENTION

The invention provides self-replicating, infectious, recombinant paramyxoviruses (PIV), methods of making the paramyxoviruses of the invention, and uses thereof. The PIV of the invention may be a complete virus, or may be a subviral particle comprising at least nucleocapsid (N), phosphoprotein (P) and large polymerase (L) proteins and a genomic or antigenomic RNA comprising all of the cis-acting sequences necessary for replication of the subviral particle and transcription and translation of the proteins encoded by the packaged genome and for packaging of the replicated genome into another viral or sub-viral particle.

The PIV of the invention may have a genome or antigenome in which the genes encoding P and C proteins, which normally overlap and are expressed from two ORFs of a common polynucleotide sequence, have been genetically separated into two polynucleotides, each providing only a single “substantial open reading frame” (SSRF) so that mutations can be independently introduced into either or both of the P and C genes. Separation of the two ORFs of the P/C gene allows intentional introduction of mutations and other variations in one ORF without deleterious effects on the other ORF. For example, the IFN-antagonist function or another function of the C proteins can be inhibited by introducing mutations into the C ORF without affecting the P protein. In this manner, the viral RNA dependent RNA polymerase activity can be maintained and virus growth in vitro is unaffected by C ORF mutations.

The PIV of the invention can also have one or more amino acid or nucleic acid mutations that confer an attenuated phenotype. In instances where mutations are made by insertions or deletions, the number of nucleotides inserted or deleted is such that the total number of nucleotides in the variant viral genome is divisible by six (known as the “rule of six”). Mutations can be stabilized by at least two changes in the codon specifying the mutation. The PIV of the invention can be human parainfluenza virus (HPIV), such as for example HPIV1 or HPIV3. The “background” genome into which mutations are introduced according to the invention may be that of HPIV1, the “wild-type” nucleotide sequence of which is represented in SEQ ID NO:1, or HPIV3, the wild-type nucleotide sequence of which is represented by SEQ ID NO: 21.

Furthermore, the PIV of the invention can be a “chimeric” PIV, having a genome in which one or more genes or genome segments of a different PIV are introduced, either as an additional gene beyond the normal complement of genes in the native viral genome (a so-called “supernumerary” gene) or as an exchange, replacing the counterpart of the gene or gene segment from the wild-type genome. The PIV of the invention can also include one or more supernumerary genes or gene segments from another source, such as a gene encoding a protein that affects the host immune response or a gene encoding an epitope from a non-PIV pathogen, such as another virus or a bacterium or parasite.

The PIV of the invention can minimally include a phosphoprotein (P), major nucleocapsid (N) protein, and/or large polymerase (L) protein. The proteins may be variant or naturally occurring. One or more of these proteins may carry one or more mutations that provide for attenuation of replication of a virus or sub-viral particle according to the invention in vivo or that provide for favorable properties of the virus during growth in vitro.

In some embodiments, the PIV of the invention comprises a partial or complete polyhexameric genome or antigenome comprising a polynucleotide having a single substantial ORF encoding a P protein and a polynucleotide having a single substantial ORF encoding one or more of the C proteins. In this context and throughout this patent application, a “SSRF polynucleotide” is a polynucleotide that has only one substantial reading frame that is transcribed from a genomic (or antigenomic) nucleotide sequence. A SSRF expresses primarily one transcript as a true and exact copy of the genomic or antigenomic nucleotide sequence. Shifted reading frames of the transcript are completely or largely silenced. A SSRF may give rise to one or more gene products that begin from different translational start sites and/or end at different translation stop sites within the SSRF. A polynucleotide that includes two shifted, expressed ORFs, such as for example the naturally occurring C/P gene of HPIV1 or HPIV3, is not a SSRF polynucleotide. However, a SSRF polynucleotide may encompass instances, such as the P/D/V gene complex of HPIV3, wherein additional transcripts are generated by RNA editing.

A “substantial reading frame” (“substantial RF”) is an ORF that encodes one or more complete viral proteins. A “complete viral protein” can include minor internal deletions or insertions of one, two or a few amino acids, but translation of the viral protein will end at the native stop codon. An ORF into which stop codons have been introduced so as to terminate translation of a protein in order to ablate expression of that protein is not a substantial open reading frame for that protein.

In some embodiments, the PIV of the invention comprises a partial or complete polyhexameric genome or antigenome comprising a first SSRF polynucleotide expressing one or more C proteins and a second SSRF polynucleotide expressing a P protein. Thus, mutations can be introduced into one or more of the C proteins without at the same time affecting the P protein, and vice versa. The PIV of the invention can comprise a partial or complete polyhexameric genome or antigenome comprising a polynucleotide encoding truncated C proteins and a SSRF polynucleotide encoding a P protein. The PIV of the invention can comprise a partial or complete polyhexameric genome or antigenome in which a SSRF polynucleotide encodes a P protein and any polynucleotide encoding C proteins is deleted.

The polynucleotide encoding a P protein and the polynucleotide encoding a C protein can be separated by a non-coding polynucleotide spacer sequence and, optionally, can be on separate vectors for use in cells for producing stocks of infectious particles. In some embodiments, the non-coding spacer comprises a gene end transcription signal, intergenic transcription signal, and gene start transcription signal. The non-coding spacer can be upstream of the P ORF. The ordering of the polynucleotides encoding the P protein and C proteins can also be changed to P followed by C, and the spacer polynucleotide can be upstream of the polynucleotide encoding the C proteins.

The polynucleotide encoding the C proteins can encode a variant C protein containing a mutation that inhibits the functions of one or more of the C proteins, e.g. ability to interrupt production of IFN in an infected host or signaling though an IFN receptor. The mutation can be amino acid or nucleic acid substitution(s) or deletion(s). In instances of insertions or deletions, the number of nucleotides inserted or deleted is such that the total number of nucleotides in the variant viral genome is divisible by six. Point mutations can be stabilized by at least two changes in the codon specifying the mutation. In some embodiments, the C protein comprises one or more amino acid substitutions at amino acid residues corresponding to codon positions 83, 84, 85, 169, 170 or 171 or deletions of one or more of these amino acids.

The SSRF polynucleotide encoding the P protein can comprise a P ORF having one or more nucleotide substitutions wherein the substitution introduces one or more stop codons in an overlapping C ORF reading frame but does not alter the amino acid sequence of P protein encoded by the SSRF polynucleotide encoding the P protein. Alternatively, or in addition, the SSRF polynucleotide encoding the P protein can comprise a P ORF having one or more nucleotide substitutions wherein the substitution destroys the start codon in an overlapping C ORF reading frame but does not alter the amino acid sequence of P protein encoded by the SSRF polynucleotide encoding the P protein.

The polynucleotide encoding one or more C proteins can comprise a C ORF having one or more nucleotide substitutions wherein the substitution introduces one or more stop codons in an overlapping P ORF but does not alter the amino acid sequence of C proteins encoded by the polynucleotide encoding the C protein. Alternatively, or in addition, the polynucleotide encoding the C protein can comprise a C ORF having one or more nucleotide substitutions wherein the substitution destroys the start codon in an overlapping P ORF reading frame but does not alter the amino acid sequence of any C protein encoded by the polynucleotide encoding the C proteins. Such polynucleotides encoding the C protein can also include one or more point mutations or deletions that reduce or abolish one or more functions of the C proteins, e.g., the ability of the C proteins to inhibit production of IFN by an infected host cell or IFN signaling as described above.

The polynucleotide encoding one or more C proteins can be a SSRF polynucleotide, or can be a polynucleotide that has at least 85% identity to SEQ ID NO:14 (wild type PIV1 C′ protein) or to SEQ ID NO: 23 (PIV3 C′ protein) and having at least one nucleotide change that ablates or reduces expression of said C protein or that expresses a truncated C protein.

Due to physical separation of the P and C genes, the mutation(s) introduced into a polynucleotide encoding the C proteins ORF will not alter the P protein. This is important since unwanted mutations of the P protein could have deleterious effects on the biological properties of the recombinant virus, such as restricted growth in vitro.

The invention also includes polynucleotides and methods of using polynucleotides. In some embodiments, an isolated nucleic acid comprises a polynucleotide having at least 85% sequence identity to a polynucleotide of SEQ ID NO: 14 (wild type HPIV1 C′ ORF) or to SEQ ID NO: 23 (wild type HPIV3 C ORF). In other embodiments, an isolated nucleic acid comprises a polynucleotide having a sequence of SEQ ID NO: 15 (mutated C′ ORF eliminating P). In further embodiments, an isolated nucleic acid comprises a polynucleotide having a sequence of one of SEQ ID NOs: 2 through 8 (cDNAs for viruses 1-7, FIG. 3). The invention also includes an isolated nucleic acid comprising a polynucleotide encoding a polypeptide having at least 85% sequence identity to a C polypeptide of SEQ ID NO: 17 (wild-type C′ protein of HPIV1) or to SEQ ID NO: 23 (wild type C protein of HPIV3). Other embodiments include an isolated nucleic acid comprising a polynucleotide encoding a polypeptide having at least 85% sequence identity to a polypeptide of SEQ ID NO: 11 (wild-type P protein of HPIV1) or to SEQ ID NO: 22 (wild type P protein of HPIV3). The invention also includes vectors including any of the polynucleotides as well as a partial or complete genome or antigenome. Also provided are methods of producing a paramyxovirus particle or sub-viral particle by culturing a host cell comprising any of the polynucleotides described herein.

The invention also encompasses incorporation of any one or more of the many known mutations from negative stranded RNA viruses into homologous positions of the HPIV1 genome that provide for a cold-passage (cp), temperature sensitive (ts), and/or attenuated (att) phenotype. For example, known mutations in the L protein of HPIV1 include mutation at one amino acid residue corresponding to position 456, 942, 992, 1710, or 1711 of an L protein having an amino acid sequence of SEQ ID NO: 13 (wild-type L protein sequence of HPIV1). The L protein may be modified by one or more of the following amino acid substitutions: position 456 is substituted with L, position 942 is substituted with A or T, position 992 is substituted with C or N, or deletion of positions 1710 and 1711. These, and some additional mutations that are useful for attenuating a PTV of the invention, are shown in Table 1 and 2:

TABLE 1
Attenuating mutations in the P/C and L genes of HPIV1 and HPIV3

| | | | | | # nt. | Growth | | |
|---|---|---|---|---|---|---|---|---|
| | | | | ORFs | reversion | restriction | Attenuation in vivo | |
| Mutation | | Gene | Virus | Affected | to wt aa | in vitro | Hamsters | AGMs |
| 1 | Δ10-15 | P/C | HPIV1 | P/C′/C/Y1/Y2 | 18 | no | yes | no |
| 2 | R84G | P/C | HPIV1 | P/C′/C/Y1/Y2 | 1 | no | yes | no |
| 3 | Δ83-84 | P/C | HPIV1 | P/C′/C/Y1/Y2 | 6 | yes | — | — |
| 4 | Δ84-85 | P/C | HPIV1 | P/C′/C/Y1/Y2 | 6 | yes | — | — |
| 5 | F170S | P/C | HPIV1 | C′/C/Y1/Y2 | 1 | no | yes | yes |
| 6 | Δ170 | P/C | HPIV1 | P/C′/C/Y1/Y2 | 6 | no | yes | yes |
| 7 | I96T | P/C/D/V | HPIV3 | P/C | 1 | no | yes | — |
| 8 | F164S | P/C/D/V | HPIV3 | P/C | | no | yes | yes |
| 9 | F456L | L | HPIV1 | L | 2 | yes | yes | yes |
| 10 | Y942H | L | HPIV1 | L | 1 | yes | yes | yes |
| 11 | Y942A | L | HPIV1 | L | 3 | yes | yes | yes |
| 12 | Y942T | L | HPIV1 | L | 3 | yes | yes | — |
| 13 | L992F | L | HPIV1 | L | 1 | no | no | yes |
| 14 | L992C | L | HPIV1 | L | 2 | yes | yes | yes |
| 15 | L992N | L | HPIV1 | L | 2 | yes | yes | — |
| 16 | L1558I | L | HPIV1 | L | 2 | yes | yes | yes |
| 17 | Δ1710-11 | L | HPIV1 | L | 6 | yes | yes | yes |
| 18 | 456 | L | HPIV3 | L | 2 | yes | no | — |
| 19 | Y942H | L | HPIV3 | L | 1 | yes | yes | — |
| 20 | L992F | L | HPIV3 | L | 1 | yes | yes | — |
| 21 | T1558I | L | HPIV3 | L | 2 | yes | yes | — |

“—” means not tested.

The PIV of the invention comprises one or more attenuating mutations. The attenuating mutations(s) can be temperature sensitive. Replication of PIV of the invention comprising one or more temperature sensitive mutations is attenuated in vitro at about 35° C. or greater, as compared to the corresponding wild type PIV. Temperature sensitive mutations can comprise amino acid substitution or deletion of one or more amino acid residues corresponding to position 456, 942, 992, 1710 or 1711 of an L protein having an amino acid sequence of SEQ ID NO: 13 (wild-type L of PIV1). In an embodiment, the substitution comprises F456L. In an embodiment, the substitution comprises Y942A or Y942T. In an embodiment, the substitution comprises L992C or L992N. In an embodiment, amino acid residues at positions 1710 and 1711 are deleted.

The attenuating mutation(s) can be non-temperature sensitive. Non-temperature sensitive mutations include a nucleotide substitution at a position corresponding to amino acid position 170 of the HPIV1 C protein, for example F170S. The polynucleotide encoding C proteins may have a nucleic acid sequence of SEQ ID NO: 16 (nucleotide sequence of F1705 C ORF). Non-temperature sensitive mutations can be host range restricted. For example, the PIV of the invention might replicate well in hamsters but might be restricted in replication in African green monkeys.

The PIV of the invention can comprise at least one temperature sensitive mutation and at least one non-temperature sensitive mutation. In an embodiment, at least one of the temperature sensitive mutations comprises an amino acid substitution or deletion of one or more amino acid residues corresponding to position 456, 942, 992, 1710, or 1711, or mixtures thereof in an L protein having an amino acid sequence of SEQ ID NO: 13 (wild-type L of HPIV1) and at least one of the non-temperature sensitive mutations comprising a nucleic acid substitution at a position corresponding to amino acid position 170 of the HPIV1 C protein.

In some embodiments, the PIV of the invention comprises one or more supernumerary heterologous polynucleotides or genome segments encoding one or more antigenic determinants of a heterologous pathogen. The heterologous pathogen can be HPIV1, HP1V2, HPIV3, measles virus, subgroup A or subgroup B respiratory syncytial virus, mumps virus, human papilloma virus, type 1 or type 2 human immunodeficiency virus, herpes simplex virus, cytomegalovirus, rabies virus, Epstein Barr virus, filovirus, bunyavirus, flavivirus, alphavirus, human metapneumovirus, or influenza virus. In an embodiment, the antigenic determinant comprises measles HA, HPIV1 HN or F (e.g. for a HPIV3 background), 1-1PIV2 FIN or F, HPIV3 HN or F (e.g. for a HPIV1 background) or respiratory syncytial virus (RSV) F or G protein. The antigenic determinant can also be one from an antigen of a bacterial pathogen, or a parasite.

In some embodiments, the supernumerary polynucleotide or genome segment can include an ORF of an immunomodulatory gene, for example an interleukin or a colony stimulating factor or other cytokine or chemokine. Such genes are well-known in the art.

Another aspect of the invention includes methods of making the PIV and polynucleotides of the invention. In some embodiments, the methods of the invention comprise removing a multicistronic polynucleotide encoding P and C proteins from the viral genome or antigenome of a PIV and inserting a polynucleotide encoding one or more C proteins, which is optionally a SSRF polynucleotide, and a SSRF polynucleotide encoding a P protein into a full length or partial genome or antigenome of a PIV. In embodiments used in cell culture, the polynucleotide encoding one or more C proteins and the SSRF polynucleotide encoding a P protein can be on the same vector or separate vectors. In some embodiments, the polynucleotide encoding a P protein and the polynucleotide encoding one or more C proteins are separated by a non-coding polynucleotide spacer sequence comprising a gene end transcription signal, intergenic transcription signal, and gene start transcription signal. The SSRF polynucleotide encoding a P protein may include various attenuating mutations affecting replication of the virus in vivo or in vitro.

In an embodiment, the removing step comprises introducing unique restriction enzyme recognition sequences into the genome or antigenome such that the recognition sequences flank the polycistronic polynucleotide, and digesting the genome with one or more restriction enzymes that cut the genome at the restriction sites flanking the polycistronic polynucleotide. In an embodiment, the inserting step comprises inserting at the cleaved restriction sites a polynucleotide encoding one or more C proteins and having at least one nucleotide change that ablates or reduces expression of said C protein(s) or that expresses a truncated C protein or that expresses a C protein with reduced activity compared to the corresponding native C protein, and a second, SSRF polynucleotide encoding a P protein and relegating the genome or antigenome. In an embodiment, the inserting step comprises inserting a SSRF polynucleotide encoding at least one C protein and a SSRF polynucleotide encoding a P protein at the cleaved restriction sites, and religating the genome or antigenome. The SSRF polynucleotide encoding a P protein may include various attenuating mutations affecting replication of the virus in vivo or in vitro.

In some embodiments, the methods of the invention comprise coexpressing in a cell an expression vector comprising a partial or complete polyhexameric genome or antigenome encoding a PIV of the invention and one or more polynucleotides encoding a N protein, a P protein and a L protein, and incubating the cell under conditions that allow for viral replication. The cells can be, for example, BHK-T7 cells, HEp-2 cells, Vero cells, or LLC-MK2 cells. Methods for obtaining viral or sub-viral particles from such cultures are considered known in the art.

Another aspect of the invention is a composition comprising PIV of the invention. The PIVs of the invention are useful, for example, in immunogenic compositions for eliciting an immune response in an animal, including live virus vaccines (which are typically attenuated live virus vaccines) and/or vectors for expressing heterologous antigens. PTV of the invention can be combined with viruses of other PIV serotypes, strains, or genera in a composition to elicit an immune response against multiple genera, serotypes, and strains.

The compositions of the invention comprise an immunogenic effective amount of a PIV of the invention and a physiologically acceptable carrier. The compositions of the invention can also comprise an adjuvant. In an embodiment, the composition of the invention comprises PIV from two or more serotypes. In an embodiment, at least one or more of the serotypes is HPIV1, HPIV2, HPIV3, or HPIV4. The HPIV2 can be strain V94, V98, or Greet In an embodiment, the composition of the invention comprises PIV from two or more genera. In an embodiment, at least one genus is Respirovirus genus.

Another aspect of the invention is methods of eliciting an immune response in an animal. The methods generally comprise administering an immunogenic effective amount of a composition of the invention to the animal. Preferably the immune response produces anti-PIV antibodies that are protective. In an embodiment, the antibodies are IgA. In an embodiment, the immune response produces antibodies that bind one or more antigenic determinants of a heterologous pathogen encoded by a supernumerary gene or genome segment of the PIV of the invention. The heterologous pathogen can be HPIV1 (to a HPIV3 background genome or antigenome), HPIV2, HPIV3 (to a HPIV1 background genome or antigenome), measles virus, subgroup A or subgroup B respiratory syncytial virus, mumps virus, human papilloma virus, type 1 or type 2 human immunodeficiency virus, herpes simplex virus, cytomegalovirus, rabies virus, Epstein Barr virus, filovirus, bunyavirus, flavivirus, alphavirus, human metapneumovirus, or influenza virus. The heterologous pathogen can be a bacterium or a parasite, such as malaria. In an embodiment, the antigenic determinant is from measles HA, HPIV1 UN or F, HPIV2 HN or F, HPIV3 HN or F or RSV G or F.

### BRIEF DESCRIPTION OF THE DRAWINGS

FIG. 1. Schematic representation of the rHPIV1 wt P/C gene and gene products. The order of the naturally occurring HPIV1 wt genome is: 3′-N-P/C-M-F-HN-L-5′. The P/C gene is highlighted since it encodes multiple proteins from two overlapping ORFs, one encoding the phosphoprotein (P) and the second encoding a group of non-structural, non-essential, C accessory proteins (C′/C/Y1/Y2). In this setting any mutation introduced into the P/C gene has the potential to affect both the P and C proteins.

FIG. 2. Schematic representation of the rHPIV1-C+P C and P gene units and gene products. The P/C gene of HPIV1 wt has been manipulated such that the C and P ORFs have been separated into individual gene units, gene unit 1 and gene unit 2, respectively. This new viral cDNA has been termed pFLC-rHPIV1-C+P, and the new order of the genome is: 3′-N-C-P-M-F-HN-L-5′. Briefly, using site-directed mutagenesis, the new C gene unit contains nucleotide changes designed to convert the P start codon and to introduce stop codons into the P ORF such that these changes are silent in C and that only the C proteins and not the P protein are expressed from this gene unit. Conversely, the new P gene unit contains nucleotide changes designed to convert the C start codon and to introduce stop codons into the C ORF such that these changes are silent in P and that only the P protein and none of the C proteins are expressed from this gene unit. These gene units are flanked by unique restriction endonuclease digestion sites to facilitate cloning.

FIG. 3. Schematic of the rHPIV1-C+P and P/C mutants. A representation of the rHPIV1 wt genome is shown at the top and below is a list of rHPIV1-C+P viruses including the rHPIV1-C+P wt and various C mutants. The C and P gene units of the rHPIV1-C+P viruses are highlighted to show the changes that have been introduced into the C and P gene units to allow the C gene unit to express the C proteins but not the P protein and, similarly, for the P gene unit to express the P protein but not the C proteins, to produce the rHPIV1-C+P virus.

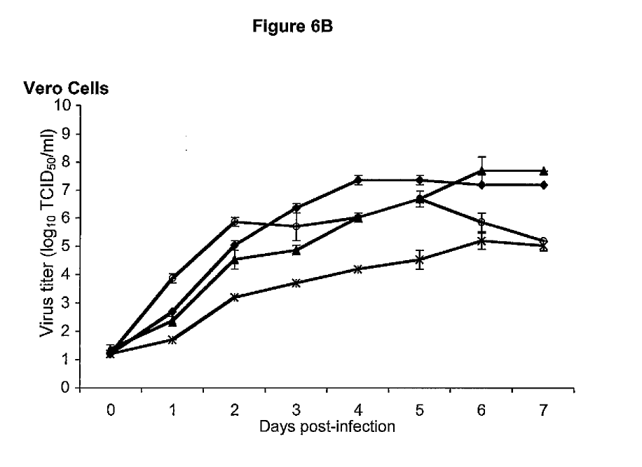

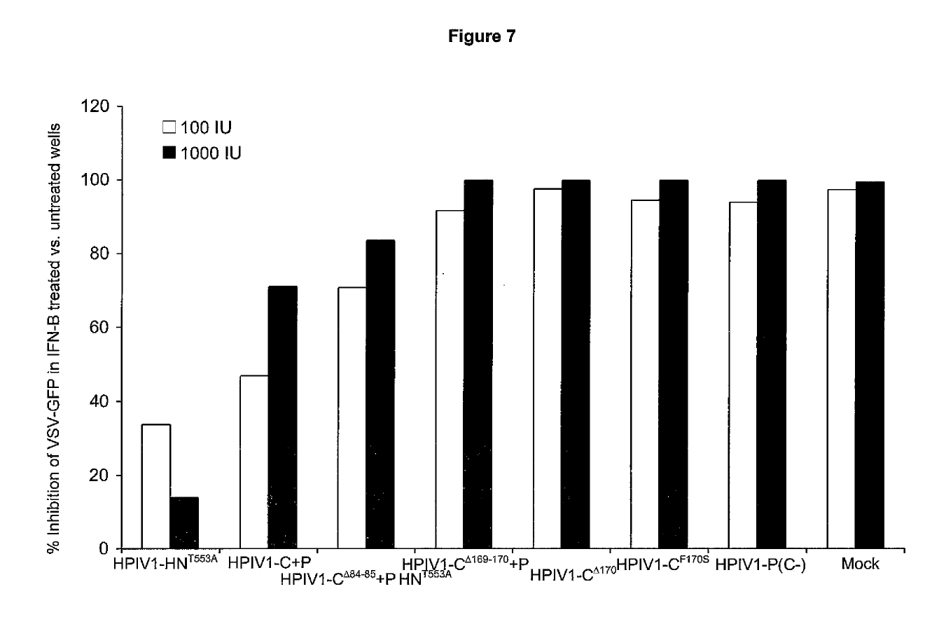

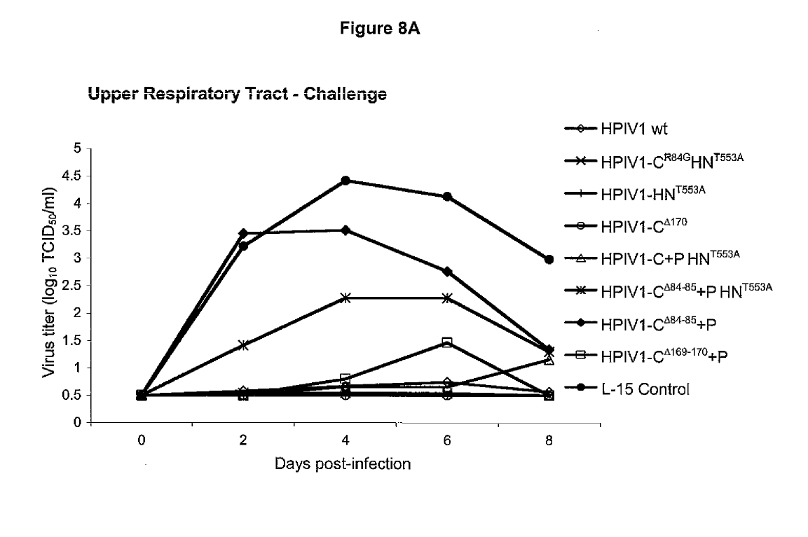

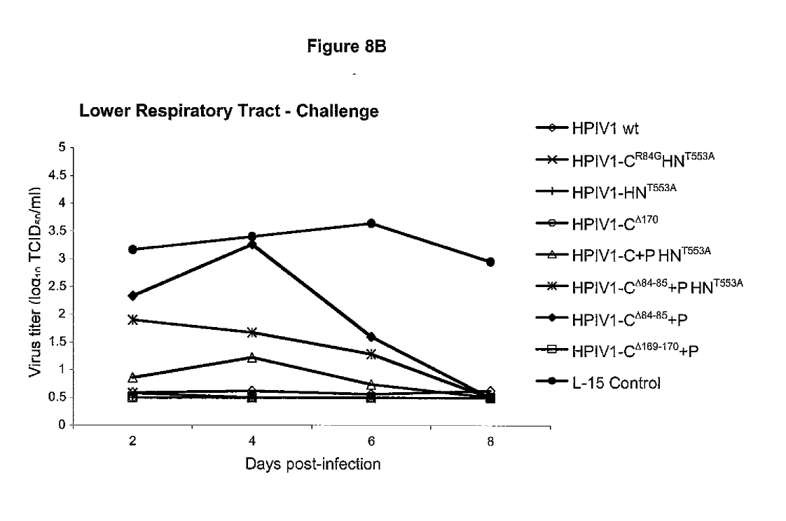

Changes include conversion of start codons (dashed lines), introduction of stop codons into the C ORF (dashed lines), introduction of stop codons into the P ORF (dotted lines) and truncation of non-coding regions (black triangles), for more detail refer to “Example 1”. Mutations that have been introduced into the C gene unit are highlighted (crosses) and include C^{Δ83-84} (rHPIV1-C^{Δ83-84}+P, virus #2, FIG. 3; Group 2, Table 1), C^{Δ84-85}+P, virus #3, FIG. 3; Group 3, Table 1), C^{Δ169-170} (rHPIV1-C^{Δ169-170}+P, virus #6, FIG. 3; Group 6, Table 1), C^{Δ170-171} (rHPIV1-C^{Δ170-171}+P, virus #7, FIG. 3; Group 7, Table 1).

In addition, rHPIV1-C+P variants that do not express any wild type C proteins have been recovered, and these include rHPIV1-C⁻(P⁻)+P (virus #4, FIG. 3) and rHPIV1-C⁻+P (virus #5, FIG. 3). The rHPIV1-C⁻(P⁻)+P mutant has been designed to include the C and P gene units of the rHPIV1-C+P, however, the C gene unit incorporates the same changes that were introduced into the P gene unit such that this gene unit

(C⁻(P⁻)) expresses neither C nor P proteins. This virus is the same length as the rHPIV1-C+P. The rHPIV1-C⁻-FP has been designed such that the C gene unit has been removed and the virus contains the P gene unit of rHPIV1-C+P such that this virus will not express C proteins. This virus is similar in length to rHPIV1 wt. “Cs stops” indicates the native stop codon that is carboxy co-terminal for all C proteins.

FIG. 4. Kinetics of replication of rHPIV1 wt and rHPIV1-C+P viruses including the C^{Δ83-84} and C^{Δ84-85} deletion mutations. Viruses including rHPIV1 wt, rHPIV1-C+P, rHPIV1-P/C^{Δ83-84}HN^{T553A}, rHPIV1-P/C^{Δ84-85}HN^{T553A}, rHPIV1^{Δ83-84}+P HN^{T553A}, and rHPIV1-C^{Δ84-85}+P HN^{T553A} were compared in a multi-cycle growth curve on LLC-MK2 cells. LLC-MK2 cell cultures were infected at an MOI=0.01 and samples were harvested for virus titration at days 0, 2, 4, 6, 8 and 9 post-infection.

FIG. 5. Map of HPIV3, organization of the HPIV3 P/C/V/D gene, and summary of modifications to construct separate C and P/V/D genes in HPIV3-C+P.

FIG. 5A shows the arrangement of the P, C, V and D ORFs in wt HPIV3, positioned according to their locations in the three reading frames (+1, +2, +3), with the codon length indicated. The P ORF is expressed by translational initiation at the ATG at positions 80-82 in the gene. C is expressed by initiation at positions 90-92. The D ORF is expressed by RNA editing at the site shown (positions 2499-2509, numbered according to the complete wt genome sequence). This shifts the reading frame and fuses the N-terminal 242 amino acids of P to 131 amino acids encoded by the D ORF, resulting in a 373 amino acid protein. The mechanism of expression of the V ORF is not known, although there is genetic evidence that it encodes a functional protein. Note that the D and V ORFs are downstream of the C ORF and are thought to be expressed independently of C. The Patel and BamHI sites (numbered according to the complete antigenome sequence) demarcate a fragment of the complete antigenome cDNA that was subcloned and engineered by the insertion of additional restriction sites and an additional gene junction as described by Skiadopoulos et al, Virology 297:136-152, 2002. The resulting cloned DNA, pUC(GE/GS)_{P-M}, provides a convenient substrate for the engineering described here.

FIG. 5B. Map of HPIV3-C+P, in which the C ORF is expressed from one SORF and the P protein is expressed from a second SORF. The D and V ORFs co-segregate with the P SSRF and would be expressed as usual from that polynucleotide.

FIG. 5C. Mutations that silence expression of the HPIV3 C ORF without affecting the P/V/D ORFs.

FIGS. 5D and 5E Mutations that silence expression of the HPIV3 P ORF, delete the V and D ORFs, and provide M/uI sites for cloning into pUC(GE/GS)_{P-M}.

FIG. 6 illustrates the kinetics of replication of rHPIV1-P(C−). FIG. 6A shows replication in LLC-MK2 cells. FIG. 6B shows replication in Vero cells. Legend is the same as for FIG. 6A.

FIG. 7 illustrates the effect of infection with rHPIV1 wild-type or mutants on type I interferon signaling.

FIG. 8 shows the efficacy of rHPIV1 wt and mutants in protecting African green monkeys (AGMs) against challenge with wild-type HPIV1. FIG. 8A shows results from the upper respiratory tract (nasopharyngeal swab). FIG. 8B shows results from the lower respiratory tract (tracheal lavage).

### DESCRIPTION OF THE INVENTION

“Paramyxovirus” as used herein refers to a paramyxovirus of the Paramyxovirinae subfamily of the Paramyxoviridae family. Paramyxoviruses are enveloped viruses that have a single strand of negative sense RNA of approximately 13 to 19 kb as a genome. Examples of paramyxovinises include, but are not limited to, human parainfluenza virus (HPIV) including types 1, 2, 3, 4A, and 4B (HPIV1, HPIV2, HPIV3, HPIV4A, and HPIV4B, respectively), mouse parainfluenza type 1 (Sendai virus, MPIV1), bovine parainfluenza virus type 3 (BPIV3), simian virus 5 (SV5), simian virus 41 (SV41), and mumps virus. HPIV1, HPIV3, MPIV1, and BPIV3 are classified in the genus Respirovirus. HPIV2, HPIV4, SV5, SV41, and mumps virus are classified in the genus Rubulavirus. MPIV1, SV5, and BPIV3 are animal counterparts of HPIV1, HPIV2, and HPIV3, respectively (Chancock et al., Parainfluenza Viruses, Knipe et al. (Eds.), pp. 1341-1379, Lippincott Williams & Wilkins, Philadelphia, 2001). HPIV1, HPIV2, and HPIV3 represent distinct serotypes and do not elicit significant cross immunity. HPIVs are etiological agents of respiratory infections such as croup, pneumonia, or bronchitis.

The term “human parainfluenza virus type 1” or “HPIV1” refers to an isolate, clone, recombinant, or variant of human parainfluenza virus type 1 of the Paramyxovirinae subfamily. A “naturally occurring” isolate or “wild type” HPIV1 is a virus isolated from a natural source or has the sequence of a HPIV1 isolated from a natural source. Naturally occurring isolates may differ from one another in sequence. In some embodiments, the genome of a naturally occurring isolate of HPIV1 of the invention has at least 85% nucleic acid sequence identity to SEQ ID NO: 1 (wild-type HPIV1 genome sequence); Genbank Accession No. NC_{—}003461). “Recombinant HPIV1” refers to virus derived from a polynucleotide that has been constructed to encode a HPIV1 genome or antigenome, and may include a sequence of a wild type or variant HPIV1. In some embodiments, the recombinant HPIV1 genome or antigenome is provided as part of an expression vector.

The HPIV1 genome encodes at least seven polypeptides. The ribonucleocapsid—associated polypeptides include the nucleocapsid protein (N) (encoded by nucleotides 120-1694 of SEQ ID NO: 1), the phosphoprotein (P) (SEQ ID NO:11 (wild-type HPIV1 P protein)), and the large polymerase (L) protein (SEQ ID NO:13 (wild-type HPIV1 L protein)) that carry out transcription and replication. The P/C gene of HPIV1 includes an alternative open reading frame (ORF) with several alternative translational start sites encoding a set of up to four C-terminal nested proteins, the C′, C, Y1, and Y2 proteins that appear to function, amongst several functions, as interferon antagonists and interferon signaling antagonists. The internal matrix protein (M) and the major protective antigens, fusion glycoprotein (F) and hemagglutinin—neuraminidase glycoprotein (HN), are envelope—associated proteins. The gene order is 3′-N-P/C-M-F-HN-L-5′.

An HPIV1 or HPIV3 encoding polynucleotide can be isolated from virus obtained from infected humans or cells or can be prepared as described herein. Viral stock of HPIV1 is available from the American Type Culture Collection under the catalog number VR-94. Viral stock of HPIV3 is available from the American Type Culture Collection under the catalog number VR-93. See also, for example, WO 03/043587 and WO 98/53078.

“Variants” of HPIV refer to a virus that has a genomic sequence that differs from the sequence of a reference virus. In some embodiments, a variant may be prepared by altering or modifying the nucleic acid sequence of the viral genome by addition, substitution, and deletion of nucleotides. As discussed previously, it is preferred that variants that have a modification due to addition or deletion of nucleotides conform to the rule of six. In some embodiments, variants may be obtained by passage of a viral particle or genome in vitro in a host cell or in vivo in a non-human host. In some embodiments, the number of nucleotides inserted or deleted is such that the total number of nucleotides in the variant viral genome is divisible by six (known as the “rule of six”).

In some embodiments, the variants have a phenotype altered in at least one aspect compared to the wild-type virus. The alterations to phenotypes can include, without limitation, a change in growth characteristics in vitro or in vivo, attenuation in vitro or in vivo, temperature sensitive growth in vitro, cold adaptation, plaque size, host range restriction or a change in immunogenicity. In some embodiments, variant HPIV1 can be immunogenic and elicit protective antibodies in a mammal. The HPIV1 variants according to the invention are attenuated in vivo in some embodiments. The degree of attenuation in vivo that is often obtained is one that decreases rhinorrhea in an infected mammalian host when compared to infection with a wild-type HPIV1. Such degree of attenuation may be approximately 10- to 10.000-fold in the respiratory tract of the infected host. Differences in the degree of attenuation between the upper and lower respiratory tract may be observed. In some embodiments, attenuation of replication of the PIV of the invention in vivo is observed, without significant attenuation of replication in vitro.

In some embodiments, the background HPIV1 or HPIV3 genome or antigenome has at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater sequence identity to SEQ ID NO: 1 (wild-type HPIV1 sequence) or to SEQ ID NO: 21 (wild type HPIV3 sequence), respectively.

In some embodiments, the PIV genome or antigenome comprises a polynucleotide encoding a C protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater sequence identity to a C gene reference sequence from HPIV1 (SEQ ID NO: 14 (wild-type HPIV1 C′ nucleotide sequence)) or to a C gene reference sequence from HPIV3 (nucleotides 1794 to 2393 of SEQ ID NO:21 (wild type HPIV3 C gene sequence)).

In some embodiments, the PIV genome or antigenome comprises a polynucleotide encoding a P protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater sequence identity to a P gene sequence from HPIV1 (SEQ ID NO: 10 (wild-type HPIV1 P gene sequence)) or from HPIV3 (nucleotides 1784 to 3595 of SEQ ID NO:21 (wild-type HPIV3 P gene sequence)).

In some embodiments, the PIV genome or antigenome comprises a polynucleotide encoding an L protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater sequence identity to a reference polynucleotide sequence encoding an L protein from HPIV1 (SEQ ID NO: 12 (HPIV1 wild-type L gene sequence) or from HPIV3 (nucleotides 8646 to 15347 of SEQ ID NO: 21 (HPIV 3 wild-type L gene sequence)).

In some embodiments, the PIV genome or antigenome comprises a polynucleotide encoding a HN protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater sequence identity to a FIN gene sequence from HPIV1 (nucleotides 6903 to 8630 of SEQ ID NO: 1 (wild-type HPIV1 HN nucleotide sequence)) or to a EN gene sequence from HPIV3 (nucleotides 6806 to 8530 of SEQ ID NO: 21 (wild type HPIV3 HN gene sequence)). Some particular viruses of the present invention incorporating at least one mutation in the HN protein are rHPIV1-C^{Δ83-84}/HN^{T553A}, rHPIV1-C^{Δ84-85}/HN^{T553A} and rHPIV1-C^{Δ169-170}/HN^{T553A}. These viruses are recombinant HPIV1 having separated C and P proteins, in which the separated C protein has the indicated deletion of two codons, the separated P protein is wild-type, and the EN protein includes the T553A point mutation.

In some embodiments, the PIV genome or antigenome comprises a polynucleotide encoding a C protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater sequence identity to a C protein sequence from FIPIVI (SEQ ID NO: 17 (wild-type HPIV1 C′ protein sequence) or from HPIV3 (SEQ ID NO: 23 (wild-type HPIV3 C protein sequence)).

In some embodiments, the PIV genome or antigenome comprises a polynucleotide encoding a P protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater sequence identity to a P protein sequence from HIPIV1 (SEQ ID NO: 11 (wild-type HPIV1 P protein sequence)) or from HPIV3 (SEQ ID NO:22 (wild-type HPIV3 P protein sequence)).

In some embodiments, the PIV genome or antigenome comprises a polynucleotide encoding an L protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater sequence identity to a reference protien sequence encoding an L protein from HPIV1 (SEQ ID NO: 13 (HPIV1 wild-type L protein sequence)) or HPIV3 (SEQ ID NO: 25 (HPIV3 wild-type L protein sequence)).

In some embodiments, the PIV genome or antigenome comprises a polynucleotide encoding a EN protein having at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater sequence identity to a reference protein sequence encoding an EN protein from HP1V1 (SEQ ID NO: 18 (HPIV1 wild-type HN protein sequence)) or HPIV3 (SEQ ID NO: 24 (HPIV3 wild-type EN protein sequence)).

The term “antigenome” means a positive sense viral RNA molecule or DNA molecule complementary to the entire negative sense single stranded viral RNA genome.

A paramyxovirus that is “attenuated” or has an “att phenotype” refers to a paramyxovirus that has decreased reactogenicity and/or decreased replication in a mammal as compared to replication of a reference wild-type paramyxovirus under similar conditions of infection. In some embodiments, a paramyxovirus that is attenuated exhibits at least about 10-fold or greater decrease, more preferably at least about 100-fold or greater decrease, more preferably at least about 1000-fold or greater decrease in virus titer in the upper or lower respiratory tract of a mammal compared to non attenuated, wild type virus titer in the upper or lower respiratory tract, respectively, of a mammal of the same species under the same conditions of infection. Examples of mammals include, but are not limited to, humans, mice, rabbits, rats, hamsters, such as for example Mesocricetus auratus, and non-human primates, such as for example Ceropithecus aethiops. An attenuated paramyxovirus may display different phenotypes including without limitation altered growth, temperature sensitive growth, host range restricted growth or plaque size alteration.

“Carriers” as used herein include pharmaceutically acceptable carriers, excipients, or stabilizers, which are nontoxic to the cell or mammal being exposed thereto at the dosages and concentrations, employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

An “infectious clone” of a paramyxovirus as used herein refers to a full-length genome or portion of a genome of a paramyxovirus isolate cloned into a replicable vector that provides for amplification of the viral genome in a cell and in some embodiments, results in viral particles. In some embodiments, a portion of the paramyxovirus genome comprises a polyhexameric nucleic acid sequence encoding at least N protein, P protein, and L protein in a single replicable vector. In other embodiments, the viral genome is a full-length genome. The replicable vector provides for introduction and amplification of the viral genome in a wide variety of prokaryotic and eukaryotic cells.

The term “immunogenic effective amount” of a paramyxovirus, component thereof, or other antigenic determinant refers to an amount of a paramyxovirus, component thereof, or other antigenic determinant that induces an immune response in an animal. The immune response may be determined by measuring a T or B cell response, or by challenging an immunized animal with a virus capable of replicating in the host species. Typically, the induction of an immune response is determined by the detection of antibodies specific for paramyxovirus, a component thereof, or other antigenic determinants. An immune response is deemed to have been “induced” if, following immunization, the replication of homotypic challenge wild type virus is reduced 10-fold or more in the upper or lower respiratory tract of a mammal.

An “isolated” nucleic acid molecule refers to a nucleic acid molecule that is identified and separated from at least one contaminant nucleic acid molecule with which it is ordinarily associated in the natural source. Preferably, the isolated nucleic acid is free of association with all components with which it is naturally associated. An isolated nucleic acid molecule is other than in the form or setting in which it is found in nature.

“Percent (%) nucleic acid sequence identity” with respect to the nucleic acid sequences identified herein is defined as the percentage of nucleotides in a candidate sequence that are identical with the nucleotides in a reference paramyxovirus nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

For purposes herein, the % nucleic acid sequence identity of a given nucleic acid sequence A to, with, or against a given nucleic acid sequence B (which can alternatively be phrased as a given nucleic acid sequence A that has or comprises a certain % nucleic acid sequence identity to, with, or against a given nucleic acid sequence B) is calculated as follows:

100 times the fraction WIZ

where W is the number of nucleotides scored as identical matches by the sequence alignment program in that program's alignment of A and B, and where Z is the total number of nucleotides in B. It will be appreciated that where the length of nucleic acid sequence A is not equal to the length of nucleic acid sequence B, the % nucleic acid sequence identity of A to B will not equal the % nucleic acid sequence identity of B to A.

“Percent (%) amino acid sequence identity” with respect to the amino acid sequences identified herein is defined as the percentage of amino acid residues in a candidate sequence that are identical with the amino acid residues polypeptide reference sequence, such as for example the amino acid sequence of N protein, P protein, C protein, M protein, F protein, HN, or L protein, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, ALIGN-2, clustal C (DNASTAR) or Megalign (DNASTAR) software. Those skilled in the art can determine appropriate parameters for measuring alignment, including any algorithms needed to achieve maximal alignment over the full-length of the sequences being compared.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

As used herein, “stable” paramyxovirus refers to a paramyxovirus that has a low risk of reversion to a reference virus sequence or phenotype after passaging, infection, or selective pressure. In some embodiments, the reference sequence is the sequence of a wild-type HPIV1. Non-wild type phenotypes include without limitation, a change in growth characteristics, attenuation in vitro or in vivo, temperature sensitive growth in vitro, cold adaptation, plaque size, host range restriction or a change in immunogenicity. A virus or sub-viral particle of the invention may exhibit more than one change in phenotype compared to a corresponding wild-type HPIV. In some embodiments, a mutation is stable if it does not revert to the reference sequence or phenotype after at least 8 in vitro cell culture passages. In some embodiments, the mutation is stable if it does not revert to a reference sequence or phenotype when grown at 38-40° C. In some embodiments, the mutation is stable if it does not revert to a reference sequence or phenotype at least 10 days post-infection of a mammal. Generally, genetic stability increases as the number of nucleotide substitutions increases. For example, a codon substitution that would require 3 nucleotides changes to revert to the wild type or wild type-like codon is more stable than a codon substitution that would require only 1 nucleotide change to revert to the wild type or wild type-like codon. Deletion mutations generally confer a greater level of genetic stability than codon substitutions. For example, deletion of a codon would require insertion of 3 nucleotides to revert to wild type.

“Recombinant” in reference to a polynucleotide refers to a polynucleotide that has been derived by recombinant DNA methods and includes recombinant molecules and recombinant viruses. “Recombinant” in reference to a paramyxovirus refers to a virus that is encoded or has been produced from such a polynucleotide. “Recombinant PIV genome or antigenome” or “rPIV” refers to a polynucleotide that has been constructed to encode a PIV strain or variant, and may include a sequence of a wild type or variant PIV. In some embodiments, the recombinant PIV genome or antigenome is in the form of a cDNA. Such a cDNA can include, in addition to viral genome or antigenome sequences, polynucleotides useful in rescue of virus or sub-viral particles from cultured cells, for example a promoter for transcribing a complete PIV genome or antigenome and a ribozyme sequence that processes the ends of the transcript. (See, e.g. WO 03/043587). In some embodiments, a polynucleotide sequence encoding all or a portion of a paramyxovirus viral genome or antigenome may be isolated and combined with other control sequences in a vector. The other control sequences may be those that are found in the naturally occurring gene or from other sources. The vector provides for amplification of the recombinant molecule(s) in prokaryotic or eukaryotic cells. It also can provide for introduction into host cells and expression of one or more desired proteins from the polynucleotide. The vectors described herein for recombinant paramyxovirus sequences are introduced into eukaryotic cells and propagated under suitable conditions as known to those of skill in the art, and are introduced into animal cells and expressed under suitable conditions as known to those of skill in the art. Infection of animal hosts with recombinant PIV of the invention can be used as a method for expressing a desired protein from a heterologous polynucleotide incorporated into a recombinant PIV genome or antigenome according to the invention.

The term “replicable vector,” as used herein, refers to a nucleic acid molecule capable of transporting another nucleic acid to which it has been linked into a cell and providing for amplification of the nucleic acid. One type of vector is a “plasmid”, which refers to a circular double stranded DNA loop into which additional DNA segments may be ligated. Another type of vector is a phage vector. Another type of vector is a viral vector, wherein additional nucleic acid segments may be ligated into the viral genome. Certain vectors are capable of autonomous replication in a host cell into which they are introduced (e.g., bacterial vectors having a bacterial origin of replication and episomal mammalian vectors). Recombinant PIV of the present invention, when used with permissive host cells or when they infect a permissive host, are of this class of vectors, provided that the recombinant PIV particle or sub-viral particle includes at least a P, N and L protein in addition to a recombinant vector genome or antigenome. Other vectors (e.g., non-episomal mammalian vectors) can be integrated into the genome of a host cell upon introduction into the host cell, and thereby are replicated along with the host genome. In some embodiments, the vector is a vector that can replicate to high copy number in a cell.

The term “shut-off temperature” refers to a temperature at which the reduction of virus titer compared to its titer at a reference temperature is 100-fold greater than the reduction of wild type virus at the same temperature. In some embodiments, the reference temperature is about 32° C., and the shutoff temperature is about 39° C., more preferably 38° C. or 37° C. A determination of the shut off temperature allows a comparison of the temperature sensitivity of different virus strains or isolates and is often indicative of the level of attenuation. The lower the shutoff temperature the higher the level of attenuation of the paramyxovirus isolate or strain.

The term “transfection” as used herein refers to introducing DNA into a eukaryotic cell so that the DNA is replicable and/or expressed, either as an extrachromosomal element or by chromosomal integration. Depending on the host cell used, transfection is done using standard techniques appropriate to such cells. Methods for transfecting eukaryotic cells include polyethyleneglycol/DMSO, liposomes, electroporation, and electrical nuclear transport.

Polypeptide sequences defined herein are represented by one-letter or three letter symbols for amino acid residues as follows:

| | | | |
|---|---|---|---|
| A | ala, alanine | L | leu, leucine |
| R | arg, arginine | K | lys, lysine |
| N | asn, asparagine | M | met, methionine |
| D | asp, aspartic acid | F | phe, phenylalanine |
| C | cys, cysteine | P | pro, proline |
| Q | gln, glutamine | S | ser, serine |
| E | glu, glutamic acid | T | thr, threonine |
| G | gly, glycine | W | try, tryptophan |
| H | his, histidine | Y | tyr, tyrosine |
| I | ile, isoleucine | V | val, valine |

Strategies to generate attenuated viruses are important in the design of safe and stable viral constructs useful in an immunogenic composition. The phenotype of a viral isolate or strain may be modified to achieve a balance between attenuation of viral replication and immunogenicity of the modified variant. In some embodiments, viral replication may be decreased about 100 to 1000 fold and yet still retain immunogenicity. In some embodiments, it is desirable to generate an attenuated virus that has at least one temperature sensitive attenuating mutation and one non-ts attenuating mutation. Attenuated viruses that have more than one mutation and/or more than one phenotype can have enhanced stability.

The C proteins of paramyxoviruses are the collection of accessory proteins named as C′, C, Y1 and Y2. The C proteins are an attractive target for introduction of one or more mutations. C proteins, amongst other functions, inhibit production of α/β interferons and decrease signaling of α/β interferons through their receptors. A paramyxovirus with one or more modified C proteins should have decreased pathogenicity since it may be defective in counteracting host cell interferon response. Replication of paramyxoviruses expressing a C protein with a carboxy terminal deletion has been found to be defective in vivo and in vitro, including in Vero cells which do not express antiviral interferons α and β (Kato et al., 1997, Embo J., 16(3):578-587; Delenda et al., 1997, Virology, 228(1):55-62; Durbin et al., 1999, Virology, 261(2):319-330; Kawano et al., 2001, Virology, 284(1):99-112; He et al., 2002, Virology, 303(1):15-32; Park et al., 2003, J. Virol., 77(17):9522-9532). The above references describe assays that are useful for assessing C protein function. The C proteins might also have other functions during viral infection, and mutations might also achieve an attenuating effect by interfering with additional functions.

In wild-type HPIV1 and HPIV3, C proteins are encoded by a polycistronic polynucleotide encoding both the P protein and the collection of C proteins. The polycistronic polynucleotide encodes a nucleocapsid-associated P phosphoprotein from an overlapping reading frame (Ohgimoto et al., 1990). P protein is a structural protein that plays a major role in transcription and replication of the viral genome. The alternative reading frames containing the C proteins (C′, C, Y1 and Y2) contain several alternative translational start sites. The overlapping nature of the P and C ORFS, however, greatly restricts the number and types of mutations that can be introduced into the C proteins without also affecting the P protein, and vice versa.

One aspect of the invention includes polynucleotides, vectors and a viral construct comprising a polynucleotide encoding a P protein and a SSRF polynucleotide encoding one or more C proteins. Separation of the coding sequence of the C proteins and the P protein allows changes to the C proteins without affecting the function of the P protein. Preferably, the polynucleotide encoding the one or more C proteins is modified with at least one mutation that decreases the ability of at least one of the C proteins to inhibit interferon production and/or signaling. The polynucleotide encoding a P protein and the polynucleotide encoding the C protein can be on the same vector or separate vectors.

Another aspect of the invention includes polynucleotides, vectors and viral constructs comprising a polynucleotide encoding one or more variant C proteins and a SSRF polynucleotide encoding a P protein. Separation of the coding sequence of the C and P proteins allows changes to the P protein without affecting the function of the C protein. In an embodiment, the polynucleotide encoding the P protein is modified with at least one mutation that destroys the start site of the C and/or C′ protein and/or truncates the C, C′, Y1 and/or Y2 proteins. The polynucleotide encoding variant C proteins and the SSRF polynucleotide encoding the P protein can be on the same vector or separate vectors.

Another aspect of the invention involves a novel attenuating mutation of the L polymerase. In some embodiments, residue positions are selected for substitution based on a comparison to other related viruses and an indication that when an amino acid at the position is substituted in other related viruses an attenuating phenotype is observed. The amino acids selected for substitution at those positions are most often chosen from those amino acids that are encoded by a codon that differs in at least two nucleotide positions from the wild type amino acid found at that position and that confer an attenuation phenotype. Thus, in some embodiments, at least two nucleotide changes are made in a codon specifying the changed amino acid. In some embodiments, mutations of the L polymerase have a temperature sensitive phenotype.

The attenuating mutations and methods of the invention provide recombinant, infectious, self-replicating paramyxoviruses comprising a partial or complete polyhexameric genome or antigenome having a SSRF polynucleotide encoding a P protein, which can include attenuating mutations, and a polynucleotide encoding C proteins, as well as additional attenuating mutations in the L polymerase and non-coding portions of the genome. Alternatively, the attenuating mutations and methods of the invention provide recombinant, infectious, self-replicating paramyxoviruses comprising a partial or complete polyhexameric genome or antigenome having a polynucleotide encoding at least one variant C protein and a SSRF polynucleotide encoding a P protein, as well as additional attenuating mutations in the L polymerase and perhaps also in non-coding portions of the genome. In some embodiments, an attenuated paramyxovirus has a temperature sensitive mutation and at least one other attenuating mutation that provides a phenotype including host range restriction, reduced plaque size, or change in immunogenicity. The attenuated infectious virus can be utilized in live virus vaccines and/or in immunogenic compositions to protect against HPIV infection and/or to deliver heterologous antigens or to deliver genes for expressing a desired heterologous protein. The attenuating mutations can be utilized as part of a menu of attenuating mutations to develop paramyxovirus strains having effective degrees of attenuation in vivo yet retaining sufficient replication in vitro that may be utilized in vaccines.

One aspect of the invention provides a recombinant infectious variant of a PIV having one or more attenuating mutations in the L polymerase (L protein). The variant PIV are most often variant of HPIV1 or of HPIV3. In some embodiments, residue positions are selected for substitution based on a comparison to other related viruses and an indication that when an amino acid at a position is substituted in other related viruses an attenuating phenotype is observed. The amino acids selected for substitution at those positions are chosen from those amino acids that are encoded by a codon that differs in at least two nucleotide positions from the wild type amino acid found at that position. In some embodiments, at least two nucleotide changes are made in a codon specifying the changed amino acid.

In some embodiments, an attenuating mutation comprises a substitution at one or more amino acid residues corresponding to positions 456, 942, 992, 1710 or 1711 of SEQ ID NO:13 (wild-type HPIV1 L protein). Preferred amino acid substitutions include F456L, Y942A, Y942T, L992C, and L992N. Preferred deletions include deletion of positions 1710 and 1711.

The attenuating mutations are preferably stable. Amino acid substitutions that require two or three nucleotide substitutions are preferred. For example, Y942A requires three nucleotide substitution mutations (wild type TAT or TAC, variant GCG). The nucleotide substitutions encoding the preferred amino acid substitutions described above are shown in Table 1.

The attenuating mutation(s) can be temperature sensitive. In an embodiment, the L protein mutations are not attenuating for replication at a permissive temperature, such as for example 30-32° C., but are attenuating for replication at restrictive temperatures, such as for example 37° C.-40° C. In an embodiment, replication of the PIV variants is reduced at about 39° C. In some embodiments, the shut-off temperature of the PIV variants is preferably about 39° C., more preferably about 38° C., or about 37° C. Preferably, the paramyxovirus strains with mutations in L polymerase have a lower shutoff temperature than control paramyxovirus. In some embodiments, the control is a wild type virus. In other embodiments, the control is another attenuated paramyxovirus.

Preferably, the PIV variants are attenuated in vivo. In an embodiment, the HPIV1 variants exhibit reduced replication in the upper and/or lower respiratory tract of a mammal as compared to wild-type HPIV1. In an embodiment, the HPIV3 variants exhibit reduced replication in the upper and/or lower respiratory tract of a mammal as compared to wild-type HPIV3. In an embodiment, the replication is reduced at least about 10 fold, 100 fold, more preferably about 500 fold, more preferably about 1000 fold, more preferably about 1500 fold, more preferably about 2000 fold, more preferably about 3000 fold, more preferably about 4000 fold, more preferably about 5000 fold, more preferably about 6000 fold as compared to wild-type HPIV1 or HPIV3. In an embodiment, the mammal is a golden Syrian hamster (Mesocricetus auratus). In another embodiment, the mammal is an African green monkey (Cercopithecus aethiops).

The PIV variants preferably comprise a partial or complete polyhexameric genome or antigenome encoding a major nucleocapsid (N) protein, a nucleocapsid phosphoprotein (P) and a L polymerase (L) protein. PIV variants may further comprise a genome or antigenome encoding a matrix (M) protein, a fusion (F) protein and a hemagglutinin-neuraminidase (HN) protein. PIV variants, and especially HPIV1 or HPIV3 variants, that further comprise F and HN proteins are particularly useful in immunogenic compositions.

Another aspect of the invention includes an isolated nucleic acid or vector comprising a polynucleotide encoding a polypeptide with at least 80% sequence identity to an L protein having a sequence of SEQ ID NO:13 (HPIV1 wt L protein) or of SEQ ID NO:25 (HPIV3 wt L protein). An isolated polypeptide comprising at least 80% sequence identity to a L polypeptide of SEQ ID NO: 13 (HPIV1 wt L protein) or to SEQ ID NO: 25 (HPIV3 wt L protein) and preferably comprising at least one mutation of amino acid residues corresponding to positions 456, 942, 992, 1710, or 1711 of SEQ ID NO: 13 (wt L protein) is also provided.

An attenuating mutation in the L protein can be generated by standard PCR mutagenesis and molecular cloning techniques using primers of design readily apparent to one of ordinary skill in the art.

One aspect of the invention provides recombinant, infectious, self-replicating paramyxovirus comprising a partial or complete polyhexameric genome or antigenome having a polynucleotide encoding a P protein, especially a SSRF polynucleotide encoding a P protein, and a second polynucleotide encoding one or more C proteins. The SSRF polynucleotide encoding a protein has been altered to no longer encode any C protein. Another aspect of the invention provides recombinant, infectious, self-replicating paramyxovirus comprising a partial or complete polyhexameric genome or antigenome having a SSRF polynucleotide encoding one or more C proteins and a SSRF polynucleotide encoding a P protein. In such an instance one or more point mutations or minor deletions of one, two or up to ten amino acids, may be introduced into the P and/or C proteins to provide an attenuating phenotype in vivo or in vitro. The paramyxovirus of the invention preferably includes a major nucleocapsid protein (N protein), a nucleocapsid phosphoprotein (P protein), and a large polymerase protein (L protein). Paramyxovirus variants may further comprise a genome or antigenome encoding a fusion (F) protein and a hemagglutinin-neuraminidase (HN) protein. Paramyxovirus variants that further comprise F and HN proteins are useful in immunogenic compositions. In some embodiments, the SSRF polynucleotide encoding a wild-type or mutant P protein and the polynucleotide encoding one or more C proteins, which may be mutated, are on separate vectors.

The paramyxovirus can be a parainfluenza virus (PIV). A number of paramyxovirus have a polynucleotide encoding both a P and C protein including HPIV1, HPIV3, murine PIV1 (Sendai virus), and measles virus. In an embodiment, the PIV is human PIV (HPIV). Preferably, the HPIV is HPIV type 1 (HPIV1). In an embodiment, the genome or antigenome sequence is derived from a variant HPIV1 comprising at least 80% or greater nucleotide sequence identity with a HPIV1 reference sequence. In an embodiment, the reference sequence is the genomic sequence of HPIV1. In an embodiment, the genomic sequence comprises a nucleotide sequence of SEQ ID NO:1 (wt HPIV1 genome).

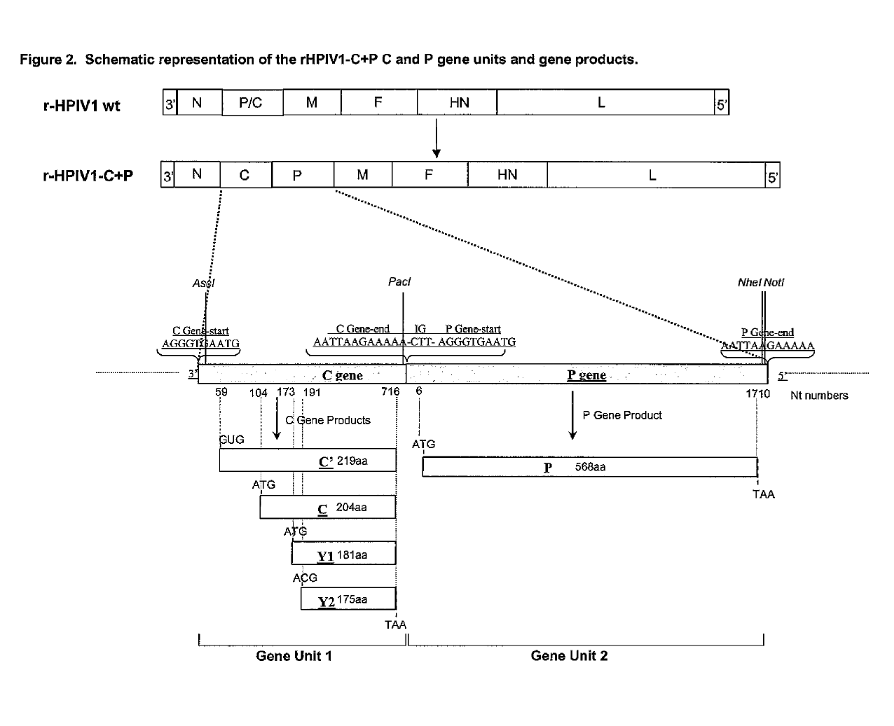

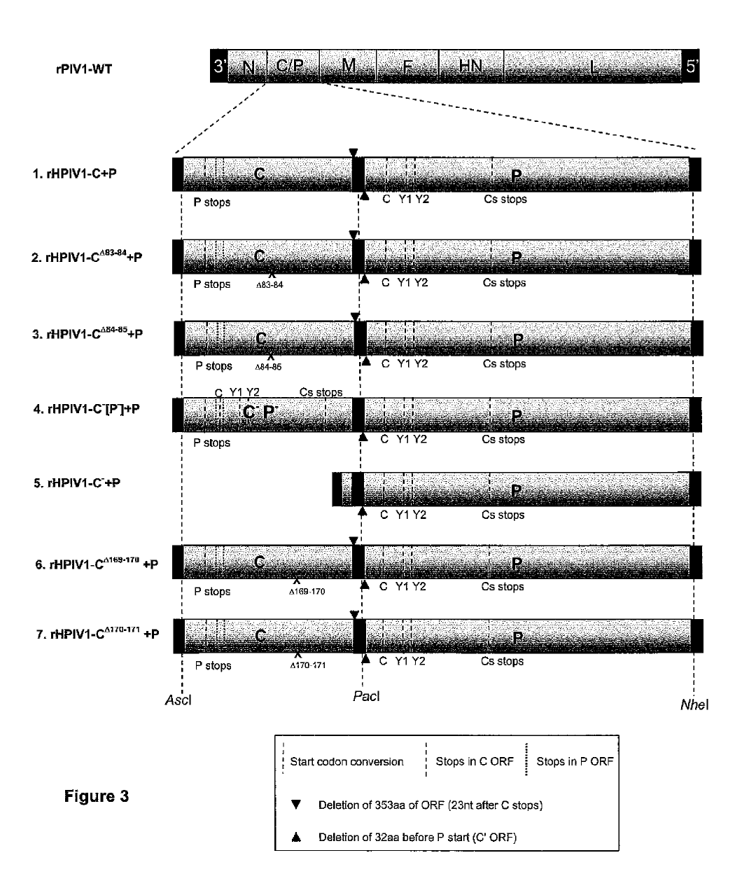

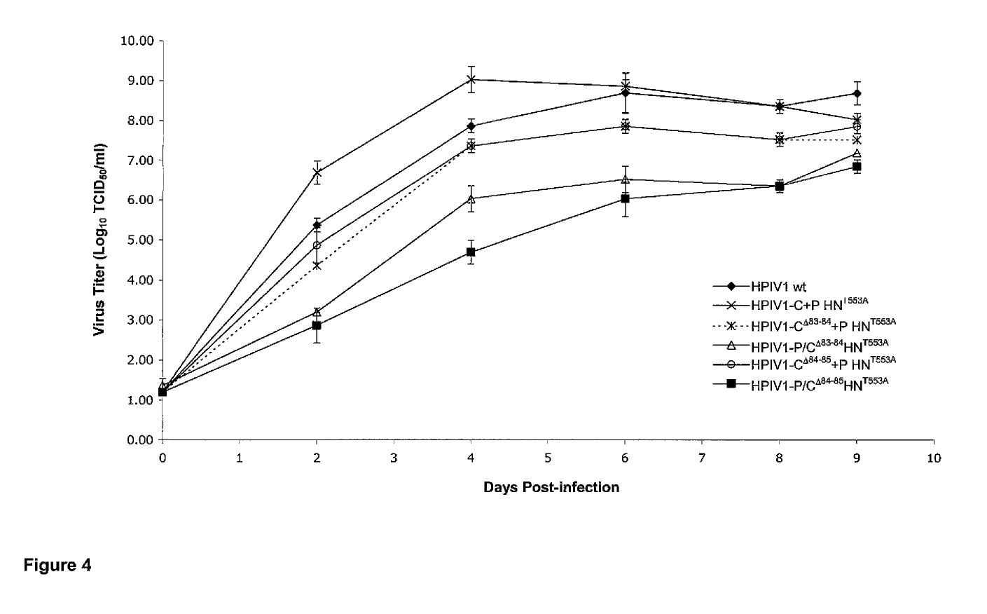

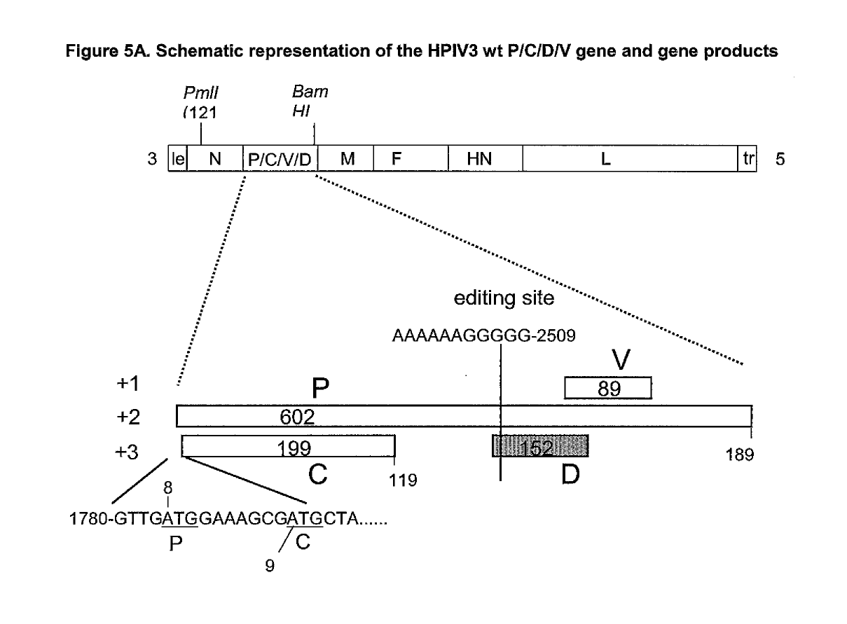

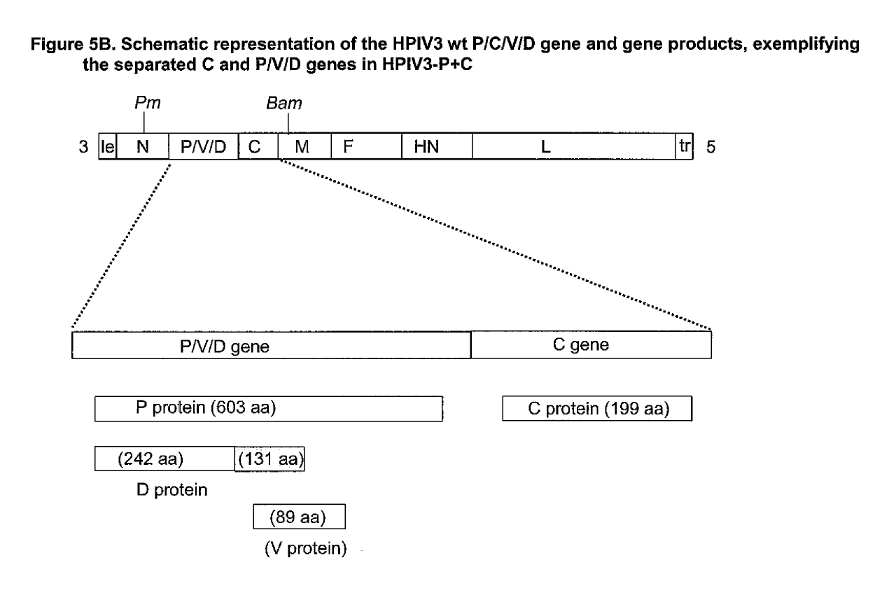

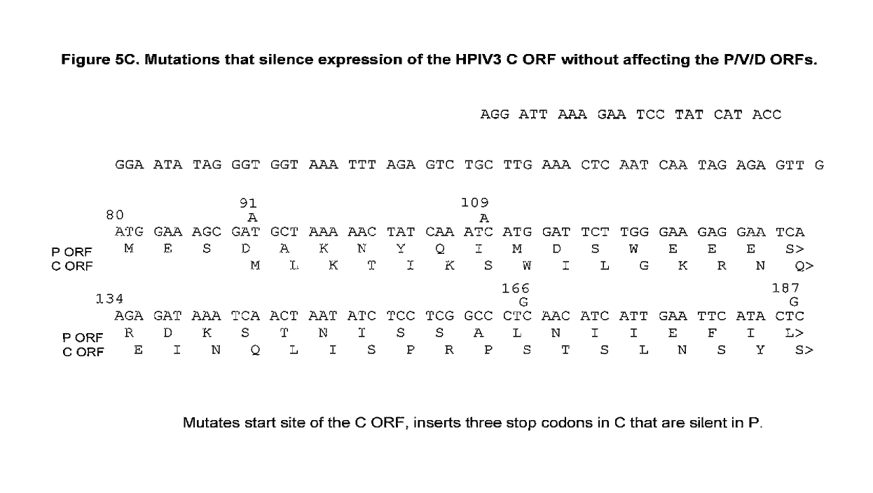

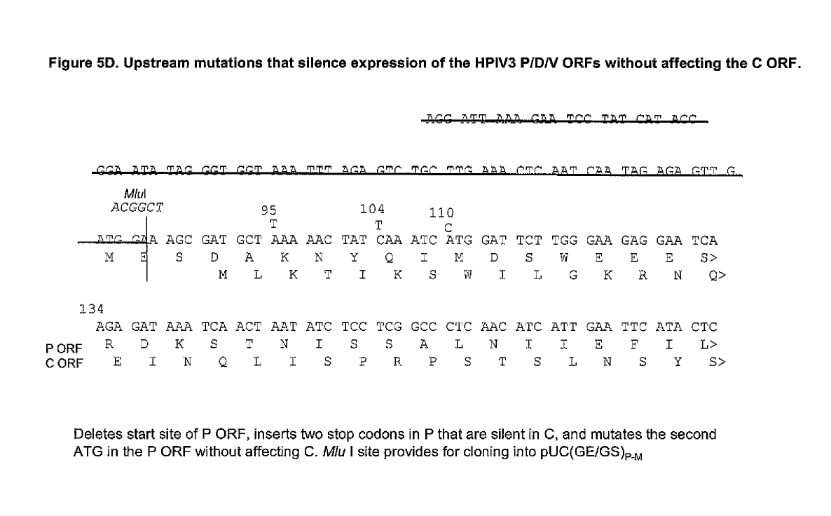

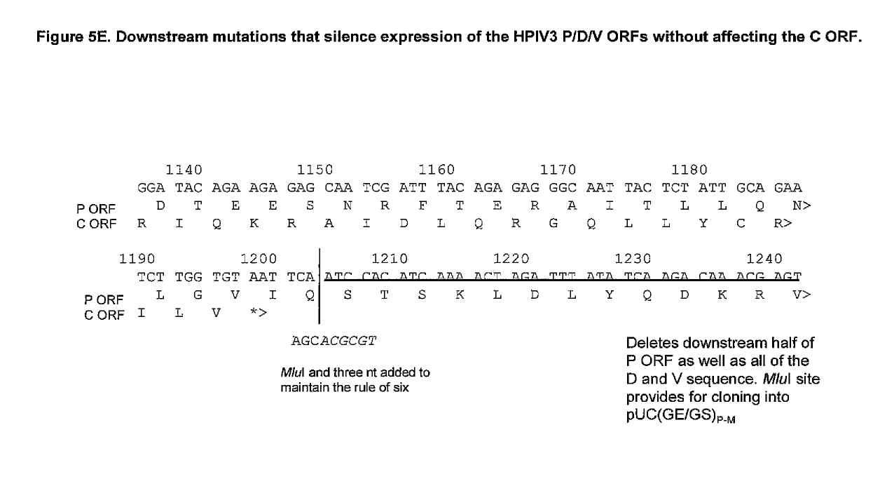

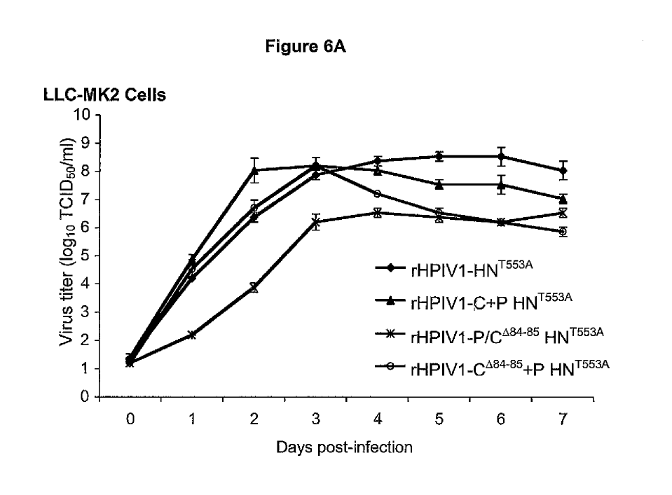

The polynucleotides separately encoding at least one C protein and the P protein can be inserted anywhere into the genome or antigenome of the recombinant PIV of the invention. In some embodiments, a SSRF polynucleotide encoding at least one C protein may be inserted at the 3′ end, and a SSRF polynucleotide encoding the P protein may be inserted elsewhere. Thus, in some embodiments, the gene order, for example, may be 3′ C-N-P-M-F-HN-L-5′. In other embodiments, the gene order, for example, may be 3′ N-C-P-M-F-HN-L-5′. In some embodiments, the SSRF polynucleotide encoding the P protein and the polynucleotide encoding at least one C protein are inserted into restriction sites in the genome. Restriction sites may include Asa Pad, NheI and NotI, as shown in FIG. 2. In an embodiment, a recombinant virus separately encoding a P and at least one C protein has an antigenomic cDNA sequence that is the complement of SEQ ID NO:15.

The SSRF polynucleotide encoding a P protein and the polynucleotide encoding at least one C protein are optionally separated by a non-coding polynucleotide spacer sequence. In an embodiment, the spacer sequence is upstream of a C coding sequence or open reading frame (ORF) in the polynucleotide encoding at least one C protein. In another embodiment the polynucleotide encoding at least one C protein and a SSRF polynucleotide encoding a P protein are optionally separated by a non-coding polynucleotide spacer sequence. In an embodiment, the spacer sequence is upstream of a P coding sequence or open reading frame (ORE) in the polynucleotide encoding the P protein. In an embodiment, the spacer sequence comprises a gene end transcription signal, intergenic transcription signal, and/or gene start transcription signal. In an embodiment, the gene start transcription signal is cis-acting. In an embodiment, the gene start transcription signal includes a first adenosine at position 6n+1. In an embodiment, the spacer sequence comprises a nucleotide sequence of SEQ ID NO:9

When a SSRF polynucleotide encoding at least one C protein is inserted into a viral genome, the inserted polynucleotide is a heterologous sequence. Recombinant viruses comprising a SSRF polynucleotide encoding at least one C protein may have an altered phenotype. In some embodiments, the phenotype may be attenuating. In some embodiments, the phenotype is temperature sensitive. Additional nucleotide changes may be introduced into a recombinant virus encoding separate P and C genes to introduce other phenotypic changes selected from a change in growth characteristics, attenuation, temperature sensitivity, cold adaptation, plaque size, host range restriction, or a change in immunogenicity.

Preferably, a recombinant paramyxovirus having separate polynucleotides encoding P and C proteins is attenuated in vivo. In an embodiment, the recombinant virus having separate polynucleotides encoding P and C proteins exhibits reduced replication in the upper and/or lower respiratory tract of a mammal as compared to wild-type HPIV1 or wild-type HPIV3. In an embodiment, the replication is reduced at least about 10 fold, 100 fold, more preferably about 500 fold, more preferably about 1000 fold, more preferably about 1500 fold, more preferably about 2000 fold, more preferably about 3000 fold, more preferably about 4000 fold, more preferably about 5000 fold, more preferably about 6000 fold as compared to wild-type HPIV1 or wild-type HPIV3. In an embodiment, the mammal is a golden Syrian hamster (Mesocrieetus auratus). In another embodiment, the mammal is an African green monkey (Cercopithecus aethiops). In some embodiments, the attenuated paramyxovirus is immunogenic and elicits sufficient antibodies to protect against infection.

The SSRF polynucleotide or variant polypeptide encoding at least one C protein can encode a C protein having a sequence of a naturally occurring or variant C protein. The C protein can be from a heterologous paramyxovirus, including but not limited to PIV3 (e.g., human or bovine PIV3, heterologous to HPIV1), HPIV1 (heterologous to HPIV3), Sendai virus, or measles virus. In an embodiment, the nucleotide sequence encoding the C protein has at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater sequence identity to a nucleotide sequence encoding the C protein of HPIV1 (SEQ ID NO:17 (wild-type HPIV1 C protein)) or of HPIV3 (SEQ ID NO: 23 (wild-type HPIV3 C protein)). In an embodiment, the antigenomic cDNA sequence encoding the C ORF is the complement of SEQ ID NO:14. In some embodiments, an isolated variant nucleic acid comprising a polynucleotide encoding a polypeptide having at least 80% sequence identity to a C protein having a sequence of SEQ ID NO:17 (wild type HPIV1 C protein sequence) or to SEQ ID NO: 23 (wild-type HPIV3 C protein sequence) is provided.

In an embodiment, the nucleotide sequence encoding at least one C protein comprises a mutation that inhibits the ability of C protein to interrupt production or signaling of interferon in an infected host or host cell. Preferably, the mutation does not substantially impact virus replication in cell culture. In an embodiment, the C protein has reduced activity as compared to the C protein expressed by wild-type HPIV1 or that expressed by wild-type HPIV3.

One of several approaches can be taken to identify mutations in a C protein that render rHPIV1 C+P attenuated for replication in vivo. The first is random mutagenesis of the C ORF to generate viruses that are restricted for replication in the respiratory tract of experimental animals. For example, alanine mutations can be introduced at each position and those positions important in the function of the protein can be identified. In other embodiments, deletions of at least 2 amino acids can be generated. Recombinant viruses bearing these mutations can be characterized in vitro and in vivo.

Alternatively, sequence alignment with heterologous paramyxovirus C proteins can be used as a guide for targeted mutagenesis. For example, there are two ways to use the sequence alignment as a guide. First, conserved sequences, which are likely required for specific C protein activities, can be directly targeted with conservative amino acid substitutions or small (2 amino acid) deletions. A less conservative approach can be taken and unrelated amino acids can be used for amino acid substitutions, or large portions of the conserved regions (6 or more amino acids) can be deleted. Unrelated amino acids may be selected that require at least two nucleotide changes in the codon as compared to the codon encoding the wild type amino acid at that position. The selection of sites for mutagenesis is not limited to conserved sequences.

Substantial modifications in the biological properties of a C protein are accomplished by selecting substitutions that differ significantly in their effect on maintaining (a) the structure of the polypeptide backbone in the area of the substitution, for example, as a sheet conformation, helical conformation, or loop structure, (b) the charge or hydrophobicity of the molecule at the target site, or (c) the bulk of the side chain. Naturally occurring residues are divided into groups based on common side-chain properties:

(1) hydrophobic: leucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions entail exchanging a member of one of these classes for another class. Such substituted residues also can be introduced into the conservative substitution sites or, more preferably, into the remaining (non-conserved) sites.

In an embodiment, the C′ and C proteins comprise one or more amino acid substitutions or deletions at or between residues corresponding to positions 83-85 of the C protein. In an embodiment, all of the C proteins comprise one or more amino acid substitutions or deletions between residues corresponding to positions 169-171 of the C protein. In an embodiment, the C proteins collectively are mutated by one or more amino acid substitutions or deletions at residues corresponding to positions 83-85 and 169-171 of the C protein. Preferably, the nucleotide changes that encode a mutation comprise at least two nucleotide changes compared to the codon encoding the wild type amino acid at that position to increase the stability of the mutation. Preferably, the variant C protein has at least 80% sequence identity to that of the wild-type C protein sequence of HPIV1 or of HPIV3.

The SSRF polynucleotide encoding a P protein includes a nucleotide sequence encoding a P protein. The SSRF polynucleotide encoding the P protein is altered so that it does not encode a C protein. The amino acid sequence of the P protein may also be a mutated sequence. In an embodiment, the nucleotide sequence encoding P protein exhibits at least 80% sequence identity, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater sequence identity to a nucleotide sequence encoding the P protein of HPIV1 (SEQ ID NO: 11 (wild-type P protein of HPIV1) or of HPIV3 (SEQ ID NO: 22 (wild type P protein sequence of HPIV3). In an embodiment, an antigenomic cDNA sequence encoding a P protein has a sequence that is the complement of SEQ ID NO:10 (wild type P gene of HPIV1).

The variant polynucleotide encoding a P protein includes a P coding sequence or open reading frame (ORF). Preferably the P ORF includes one or more nucleotide substitution(s) that introduce one or more stop codons in an overlapping C ORF but does not alter the amino acid sequence of P protein encoded by the P ORF. In an embodiment, third base codon substitutions encoding stop codons in the C ORF are introduced (see FIG. 2) into the P ORF (SEQ ID NO:15). In another embodiment, the P ORF comprises a nucleotide sequence of SEQ ID NO:10.

A paramyxovirus or polynucleotide of the invention including a SSRF polynucleotide encoding a P protein and a second polynucleotide encoding at least one C protein can be made using known recombinant methods such as oligonucleotide-mediated (site-directed) mutagenesis, alanine scanning, PCR mutagenesis, site-directed mutagenesis (Zoller et al., 1987, Nucl. Acids Res., 10: 6487-6500), cassette mutagenesis (Wells et al., 1985, Gene, 34:315), restriction selection mutagenesis (Wells et al., 1986, Philos. Trans. R. Soc. London SerA, 317:415), and the like. Similarly, a paramyxovirus or polynucleotide of the invention including a SSRF polynucleotide encoding a P protein and second polynucleotide encoding at least one C protein that is a SSRF polynucleotide can be made using these same methods.

A paramyxovirus of the invention including a partial or complete polyhexameric genome or antigenome having a SSRF polynucleotide encoding a P protein and a second polynucleotide encoding at least one C protein, which is optionally also a SSRF polynucleotide, can include any of the attenuating mutations in the L polymerase described herein or other known attenuating mutations. The polynucleotide encoding at least one C protein can also include further attenuating point mutations or deletions. Likewise, an isolated polynucleotide of the invention, including a partial or complete polyhexameric genome or antigenome having a polynucleotide, which is optionally a SSRF polynucleotide, encoding at least one C protein and a SSRF polynucleotide encoding a P protein, can include any of the attenuating mutations in the L polymerase described herein or other known attenuating mutations. In such embodiments, the polynucleotide encoding at least one C protein can also include further attenuating point mutations or deletions.

Recombinant viral variants having more than one mutation are likely to have increased stability over those variants having a single mutation or a small number of mutations (eg., less than 3 mutations). The paramyxovirus of the invention can also include any of the attenuating L polymerase mutations described in WO 04/027037 or in WO 98/53078. The attenuating effect of the mutations in the L polymerase are additive and serve to further increase the attenuation of the paramyxovirus of the invention including a SSRF polynucleotide encoding a P protein and a second polynucleotide encoding at least one C protein. In some embodiments, it is desirable to balance the level of attenuation with the immunogenicity. In preferred embodiments, the variant paramyoviruses have about 100 fold to 5000 fold decrease in viral titer in a mammal. In some embodiments, a decrease of viral replication greater than about 100,000 fold in vivo may result in a loss of immunogenicity. Such a high degree of attenuation in vitro may result in an inability to produce the virus on a large scale for vaccine production.

In some embodiments, the variant paramyxoviruses of the invention have at least one temperature sensitive mutation and at least one non-temperature senstitive mutation. In an embodiment, the recombinant variant paramyxoviruses of the invention have at least one temperature sensitive mutation due to insertion of a SSRF polynucleotide encoding at least one C gene or due to a mutation in a polynucleotide encoding a L polymerase, wherein the change in the amino acid is due to at least two nucleotide changes to the codon of the L polymerase encoding the wild type amino acid. In another embodiment, the recombinant variant paramyxoviruses of the invention comprise a mutation that provides a host range restriction phenotype.

The paramyxoviruses of the invention are also useful as vectors for expressing heterologous antigens in an immunogenic composition. One or more supernumerary genes encoding one or more heterologous polypeptides can be cloned into and expressed by the paramyxovirus of the invention. For example, an immune response against multiple PIV serotypes or strains can be elicited by engineering protective epitopes of multiple PIV serotypes and strains into a single paramyxovirus. The supernumerary genes can be cloned and expressed in a recombinant virus encoding separate P and C proteins as described herein, which may optionally include one or more mutations in L polymerase. Insertion of additional heterologous genes may also result in an attenuated phenotype. Preferably, the paramyxovirus comprising a polynucleotide encoding a heterologous gene is attenuated about 100 to 5000 fold or more in a cell or mammal.

In an embodiment, the genome or antigenome includes one or more heterologous genes or genome segments encoding one or more antigenic determinants of a heterologous pathogen. For example, one or more heterologous antigenic determinant(s) from PIV, measles virus, subgroup A and subgroup B respiratory syncytial viruses, mumps virus, human papilloma viruses, type 1 and type 2 human immunodeficiency viruses, herpes simplex viruses, cytomegalovirus, rabies virus, Epstein Ban virus, filoviruses, bunyaviruses, flaviviruses, alphaviruses, human metapneumoviruses, or influenza viruses can be expressed by the paramyxovirus of the invention. Examples of useful antigenic determinants include, but are not limited to, measles virus HA and F proteins, subgroup A or subgroup B respiratory syncytial virus F and G proteins, mumps virus HN and F proteins, human papilloma virus L1 protein, type 1 or type 2 human immunodeficiency virus gp160 protein, herpes simplex virus and cytomegalovirus gB, gC, gD, gE, gG, gH, gI, gJ, gK, gL, and gM proteins, rabies virus G protein, Epstein Barr Virus gp350 protein, filovirus G protein, bunyavirus G protein, flavivirus preM and E proteins, human metapneuomovirus (HMPV) G and F proteins, and alphavirus E protein, and antigenic domains, fragments and epitopes thereof.

In an embodiment, a polynucleotide encoding an open reading frame (ORF) of a measles virus HA gene is incorporated into a HPIV1 vector genome or antigenome to yield a chimeric candidate useful to immunize against measles and/or HPIV1 or another HPIV. In another embodiment, a polynucleotide comprising genes or genome segments encoding one or more heterologous PIV(s) (e.g., HPIV2, HPIV3, and/or HPIV4) N, P, V, F, HN and/or L protein(s) or fragment(s) thereof is incorporated into a HPIV1 vector genome or antigenome. In another embodiment, one or more supernumerary heterologous gene(s) or genome segment(s) selected from BPIV1 HN, HTIV1 F, HPIV2 HN, HPIV2 F, HPIV3 HN, HPIV3 F, measles HA and F, HMPV G and F proteins, and/or RSV subgroup A or B G and F proteins are cloned into a paramyxovirus of the invention.

Some methods of inserting one or more supernumerary genes or transcriptional units into a paramyxovirus viral genome or antigenome are described in WO04/027037, hereby incorporated by reference. Supernumerary heterologous gene(s) or genome segment(s) can be inserted at various sites within the recombinant genome or antigenome, for example at a position 3′ to N, between the N/P, P/M, and/or HN/L genes, or at another intergenic junction or non-coding region of a HPIV1 vector genome or antigenome. Preferably, the heterologous or supernumery gene or transcriptional unit is inserted at a restriction site, for example, AscI PacI NheI and NotI. Any insertions or deletions of the viral genome, preferably, conform to the rule of six.

The infectious paramyxoviruses and polynucleotides of the invention are produced by synthetic and recombinant methods. Accordingly, the invention relates to polynucleotides encoding infectious paramyxovirus clones of the invention and host cells including the infectious clone, as well as methods of making such vectors and host cells by recombinant methods.

The paramyxovirus or polynucleotides of the invention may be synthesized or prepared by techniques well known in the art. See, for example, WO 94/027037. Nucleotide sequences for wild type paramyxovirus genomes are known and readily available, for example, on the Internet at GenBank (accessible via the internet). The nucleotide sequences encoding the paramyxovirus of the invention may be synthesized or amplified using methods known to those of ordinary skill in the art including utilizing DNA polymerases in a cell free environment.

Amino acid substitutions, insertions, and deletions can be made using known recombinant methods such as oligonucleotide-mediated (site-directed) mutagenesis, alanine scanning, PCR mutagenesis, site-directed mutagenesis (Zoller et al., 1987, Nucl. Acids Res., 10: 6487-6500), cassette mutagenesis (Wells et al., 1985, Gene, 34:315), restriction selection mutagenesis (Wells et al., 1986, Philos. Trans. R. Soc. London SerA, 317:415), and the like.

The paramyxovirus of the invention can be produced from virus isolated from biological samples. The polynucleotides and vectors may be produced by standard recombinant methods known in the art, such as polymerase chain reaction (Sambrook, et al., 1989, Molecular Cloning, A Laboratory Manual, Vols. 1-3, Cold Spring Harbor Press, Cold Spring Harbor, N.Y.). Methods of altering or modifying nucleic acid sequences are also known to those of skill in the art.

The paramyxovirus genome may be assembled from polymerase chain reaction cassettes sequentially cloned into a vector including a selectable marker for propagation in a host. Such markers include dihydrofolate reductase or neomycin resistance for eukaryotic cell culture and tetracycline or ampicillin resistance genes for culturing in E. coli and other bacteria.

The polynucleotide may be inserted into a replicable vector for cloning using standard recombinant methods. Various vectors are publicly available. The vector may, for example, be in the form of a plasmid, cosmid, viral particle, or phage. The appropriate nucleic acid sequence may be inserted into the vector by a variety of procedures. In general, a nucleic acid is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors including one or more of these components employs standard ligation techniques that are known to the skilled artisan.

Examples of suitable replicable vectors include, without limitation, pUC19 or pTM1. The polynucleotide can be operably linked to an appropriate promoter such as, for example, T7 polymerase promoter, cytomegalovirus promoter, cellular polymerase II promoter, or SP1 promoter. The replicable vectors may further include sites for transcription initiation, transcription termination, and a ribosome binding site for translation.

In an embodiment, an isolated polynucleotide of the invention, comprising a polynucleotide of a partial or complete PIV genome or antigenome and including a SSRF polynucleotide encoding a P protein and a second polynucleotide, optionally also a SSRF polynucleotide, encoding at least one C protein are cloned by introducing unique restriction enzyme recognition sequences into paramyxovirus cDNA such that the recognition sequences flank the bicistronic polynucleotide encoding the P/C proteins, digesting the genome with one or more restriction enzymes that cut the genome at the restriction sites flanking the bicistronic polynucleotide encoding P/C proteins, inserting the SSRF polynucleotide encoding the P protein and/or a second polynucleotide, optionally also a SSRF polynucleotide, encoding at least one C protein at the cleaved restriction sites, and religating the genome. Examples of suitable restriction enzyme recognition sequences include but are not limited to, Asa Pad, 1Vhel and NotI. In some embodiments, the restrictions sites are introduced into the non-coding regions upstream or downstream of the bicistronic P/C ORFs. In an embodiment, an Ascl site is upstream of the bicistronic P/C ORFs and the NotI site is downstream of the bicistronic P/C ORFs (FIG. 2). In an embodiment, the variant polynucleotide encoding a P protein is introduced into the genome using the Pad, and MeI restriction sites. In an embodiment, the monocistronic polynucleotide encoding a C protein is introduced into the genome using the Asa and NotI restrictions sites.

In an embodiment, a paramyxovirus of the invention including a variant polynucleotide, which is optionally a SSRF polynucleotide, encoding at least one C protein and a SSRF polynucleotide encoding a P protein are cloned by introducing unique restriction enzyme recognition sequences into paramyxovirus cDNA such that the recognition sequences flank the bicistronic polynucleotide encoding the P/C proteins, digesting the genome with one or more restriction enzymes that cut the genome at the restriction sites flanking the bicistronic polynucleotide encoding P/C proteins, inserting the polynucleotide encoding at least one C protein and/or a SSRF polynucleotide encoding a P protein at the cleaved restriction sites, and religating the genome. Examples of suitable restriction enzyme recognition sequences, include but are not limited to, AscI, PacI, NheI and NotI. In some embodiments, the restrictions sites are introduced into the non-coding regions upstream or downstream of the bicistronic P/C ORFs. In an embodiment, an Asa site is upstream of the bicistronic P/C ORFs and the NotI site is downstream of the bicistronic P/C ORFs (FIG. 2). In an embodiment, the polynucleotide encoding at least one C protein is introduced into the genome using the AscI and NotI restriction sites. In an embodiment, the SSRF polynucleotide encoding a P protein is introduced into the genome using the PacI and NheI restrictions sites.

Introduction of a recombinant vector composed of a paramyxovirus genome or polynucleotide encoding a paramyxovirus protein into a host cell, such as for example a bacterial cell or eukaryotic cell, can be affected by calcium phosphate transfection, DEAE-dextran mediated transfection, cationic lipid-mediated transfection, electroporation, electrical nuclear transport, chemical transduction, electrotransduction, infection, or other methods. Such methods are described in standard laboratory manuals such as Sambrook, et al., 1989, Molecular Cloning, A Laboratory Manual, Vols. 1-3, Cold Spring Harbor Press, Cold Spring Harbor, N.Y. or Davis et al., 1986, Basic Methods in Molecular Biology. Commercial transfection reagents, such as Lipofectamine (Invitrogen, Carlsbad, Calif.) and FuGENE 6™ (Roche Diagnostics, Indianapolis, Ind.), are also available. In some embodiments, transfection efficiency of the host cells is about 15% or greater, about 20% or greater, about 30% or greater, about 40% or greater, or about 50% or greater. Suitable host cells include, but are not limited to BHK-T7 cells, HEp-2 cells, FRhL-DBS2 cells, LLC-MK2 cells, MRC-5 cells, and Vero cells.

The invention provides isolated, infectious, recombinant paramyxovirus including one or more attenuating mutations for use in immunogenic compositions, including live attenuated virus vaccines. The paramyxoviruses of the invention are useful in immunogenic compositions for eliciting an immune response in a mammal. The attenuated paramyxovirus includes a SSRF polynucleotide encoding a P protein and a second polynucleotide, which is optionally also a SSRF polynucleotide, encoding at least one C protein. In an embodiment, the C gene comprises a nucleotide sequence encoding at least one C protein having a mutation that inhibits the ability of the C protein to interrupt production and/or signaling of interferon in an infected host or alter another function of C that results in attenuation. In an embodiment, the attenuated virus is HPIV1.

Recombinant HPIV1 of the invention can be combined with viruses of other PIV serotypes or strains and paramyxoviruses from multiple genera in a composition to elicit an immune response against multiple genera, serotypes, and strains. The immunogenic composition can comprise paramyxoviruses from two or more serotypes. In an embodiment, at least one of the serotypes is HPIV1, HPIV2, HPIV3, or HPIV4. The immunogenic composition can comprise paramyxovirus from two or more strains. In an embodiment, at least one of the strains is an HPIV1. The immunogenic composition can comprise paramyxovirus from two of more genera. In an embodiment, one genus is Respirovirus genus.

The paramyxoviruses of the invention are also useful as vectors for expressing heterologous antigens in an immunogenic composition. One or more supernumerary genes encoding one or more heterologous polypeptides can be cloned into and expressed by the paramyxovirus of the invention. For example, an immune response against multiple PIV serotypes or strains can be elicited by engineering protective epitopes of multiple Ply serotypes and strains into a single paramyxovirus. In an embodiment, the genome or antigenome includes one or more heterologous genes or genome segments encoding one or more antigenic determinants of a heterologous pathogen.

For example, one or more heterologous antigenic determinant(s) from PIV1, PIV2, PIV3, PIV4, measles virus, subgroup A and subgroup B respiratory syncytial viruses, mumps virus, human papilloma viruses, type 1 and type 2 human immunodeficiency viruses, herpes simplex viruses, cytomegalovirus, rabies virus, Epstein Barr virus, filoviruses, bunyaviruses, flaviviruses, alphaviruses, human metapneumovituses, or influenza viruses can be expressed by the paramyxovirus of the invention. Examples of useful antigenic determinants include, but are not limited to, PIV2, 3, or 4 HN and F proteins, measles virus HA and F proteins, subgroup A or subgroup B respiratory syncytial virus F and G proteins, mumps virus FIN and F proteins, human papilloma virus L1 protein, type 1 or type 2 human immunodeficiency virus gp160 protein, herpes simplex virus and cytomegalovirus gB, gC, gD, gE, gG, gH, gI, gJ, gK, gL, and gM proteins, rabies virus G protein, Epstein Barr Virus gp350 protein, filovirus G protein, bunyavirus G protein, flavivirus preM and E proteins, human metapneuomovirus (HMPV) G and F proteins, and alphavirus E protein, and antigenic domains, fragments and epitopes thereof.

In an embodiment, a polynucleotide encoding an open reading frame (ORF) of a measles virus HA gene is incorporated into a HPIV1 vector genome or antigenome to yield a chimeric candidate useful to immunize against measles and/or HPIV1 or another HPIV. In another embodiment, a polynucleotide comprising genes or genome segments encoding one or more heterologous PIV(s) (e.g., HPIV2, HPIV3, and/or HPIV4) N, P, C, V, F, HN and/or L protein(s) or fragment(s) thereof is incorporated into a HPIV1 vector genome or antigenome. In another embodiment, one or more supernumerary heterologous gene(s) or genome segment(s) selected from HPIV2 HN, HPIV2 F, HPIV3 HN, HPIV3 F, measles HA and F, HMPV G and F proteins, and/or RSV subgroup A or B G and F proteins are cloned into a paramyxovirus of the invention.

The recombinant PIV of the invention can also incorporate one or more antigenic determinants of a non-viral pathogen such as a bacterium or a parasite. Also, the host immune response can be affected by including in the recombinant PIV of the invention a gene encoding an immunomodulatory molecule, such as an interleukin or colony stimulating factor, or any other chemokine or cytokine.

The isolated nucleic acids and recombinant PIV of the invention can also serve as vectors for introducing heterologous genes (to PIV) into a mammalian host cell or animal for expression of a desired gene.

Some methods of inserting one or more supernumerary genes or transcriptional units into a paramyxovirus viral genome or antigenome are described in WO04/027037, hereby incorporated by reference. Supernumerary heterologous gene(s) or genome segment(s) can be inserted at various sites within the recombinant genome or antigenome, for example at a position 3′ to N, between the NIP, P/M, and/or HN/L genes, or at another intergenic junction or non-coding region of a HPIV1 vector genome or antigenome.

Candidate viruses for use in an immunogenic composition, such as for example a vaccine, are selected based on their attenuation and immunogenicity. These vaccine selection criteria are determined according to well-known methods. Preferably, candidate viruses have a stable attenuation phenotype, exhibit replication in an immunized host, and effectively elicit production of an immune response in a recipient, preferably a protective immune response. Preferably, the candidate viruses stimulate and expand the immune response, e.g., induce an immune response against different viral strains or subgroups and/or stimulate an immune response mediated by a different immunologic basis (e.g. secretory versus serum immunoglobulins, cellular immunity, and the like).

Recombinant paramyxoviruses of the invention can be tested in well-known and in vitro and in vivo models to confirm adequate attenuation, resistance to phenotypic reversion, and immunogenicity. In in vitro assays, the modified virus paramyxovirus of the invention is tested for one or more desired phenotypes, such as, for example, temperature sensitive replication. Paramyxovirus of the invention can also be tested in animal models of PIV infection. A variety of animal models are known. For example, PIV model systems, including rodents and non-human primates, for evaluating attenuation and immunogenic activity of PIV vaccine candidates, are known, and the data obtained therefrom are known to correlate with PIV infection, attenuation, and immunogenicity in humans.

In some embodiments, recombinant variant paramyxoviruses have at least one attenuating mutation with a is phenotype and at least one mutation with a non-ts phenotype. The recombinant attenuated paramyxoviruses are preferably attenuated about 100 to 5000 fold in a cell or mammal compared to wild type paramyxovirus. In some embodiments, attenuation of greater than 100,000 fold may result in reduced immunogenicity. In some embodiments, it is preferred that the level of viral replication in vitro is sufficient to provide for production of viral vaccine for use on a wide spread scale. In some embodiments, it is preferred that the level of viral replication of attenuated paramyxovirus in vitro is at least 10⁶, more preferably at least 10⁷, and most preferably at least 10⁸ per ml. The attenuating mutation is preferably one that is stable. For example, for mutations in L polymerase, it is preferable that a change in amino acid at a position requires at least two nucleotide changes in the codon as compared to the codon encoding the wild type amino acid at that position. A recombinant paramyxovirus with at least two, three, four or even more attenuating mutations is likely to be more stable. Insertion of a supernumerary gene whose total length conforms to the rule of six, such as a polynucleotide encoding a separate C gene, can also provide a stable phenotype.

Immunogenicity of a recombinant attenuated paramyxovirus can be assessed in an animal model by determining the number of animals that form antibodies to the paramyxovirus after one immunization or after a second immunization. In some embodiments, a recombinant paramyxovirus has sufficient immunogenicity if about 60 to 80% of the animals develop antibodies after the first immunization and about 80 to 100% of the animals develop antibodies after the second immunization. The preferred animal for a determination of immunogenicity is African green monkey. Preferably, the immune response protects against infection with a paramyxovirus of the same strain or multiple strains.

The invention also provides for immunogenic compositions comprising isolated polynucleotides or polypeptides of the invention. For example, an immunogenic composition can include a polynucleotide encoding a polypeptide that has at least 80% sequence identity to a C polypeptide having a sequence of SEQ ID NO:17 (wild-type HPIV1 C protein) or a polypeptide having a sequence at least 80% sequence identity to a C polypeptide having an sequence of SEQ ID NO:23 (wild-type HPIV3 C protein). In other embodiments, an immunogenic composition can include a polynucleotide encoding a polypeptide that has at least 80% sequence identity to a L polypeptide having a sequence of SEQ ID NO:13 (wild type HPIV1 L protein) or a polypeptide having a sequence at least 80% sequence identity to a L polypeptide having an sequence of SEQ ID NO:25 (wild type HPIV3 L protein). In other embodiments, an immunogenic composition can include a nucleic acid comprising a polynucleotide of SEQ ID NO:13 or SEQ ID NO:25.

Recombinant paramyxoviruses of the invention are preferably present in the immunogenic composition in an immunogenic effective amount. An immunogenic effective amount is an amount of recombinant paramyxovirus that induces an immune response in an animal. The actual amount of the recombinant paramyxovirus may vary depending on the animal to be immunized, the route of administration and adjuvants. The actual amount of recombinant paramyxovirus necessary to elicit an immune response, and the timing and repetition of administration, can be determined using conventional methods based on the state of health and weight of the host, mode of administration, nature of formulation, etc. Immunogenic dosages can be determined by those of skill in the art. Dosages will generally range from about 10³ to about 10⁷ infectious units (e.g., plaque forming units or 50% tissue culture infectious dose) or more of virus per host, more commonly from about 10⁴ to 10⁶ infectious doses of virus per host. In any event, the formulations should provide a quantity of attenuated recombinant paramyxovirus of the invention sufficient to effectively stimulate or induce an anti-PIV or other anti-pathogen immune response.

The immune response may be indicated by T and/or B cell responses. Typically, the immune response is detected by the presence of antibodies that specifically bind to a particular antigen. Methods of detecting antibodies to a particular antigen are known to those of skill in the art and include such assays as ELISA assays, western blot assays, hemagglutination-inhibition assays, and infectivity neutralization assays. Host receiving immunogenic compositions of the invention are preferably monitored for signs and symptoms of upper and lower respiratory tract illness. Preferably, attenuated virus administered intranasally grows in the nasopharynx of recipients at levels about 10-fold or more lower than wild-type virus, or about 10-fold or more lower when compared to levels of incompletely attenuated virus.

In neonates and infants, multiple administrations may be required to elicit sufficient levels of immunity. Administration could begin within the first month of life, and at intervals throughout the first several years of childhood, such as at two months, six months, one year and two years, as necessary to maintain an immune response against native (wild-type) PIV infection. Similarly, adults who are particularly susceptible to repeated or serious PIV infection, such as, for example, health care workers, day care workers, family members of young children, the elderly, individuals with compromised cardiopulmonary function, may require multiple immunizations to establish and/or maintain immune responses. Levels of induced immunity can be monitored by measuring amounts of neutralizing secretory and serum antibodies, and dosages adjusted or immunizations repeated as necessary to maintain desired levels of immune response.

Recombinant paramyxoviruses, polynucleotides, and polypeptides of the invention can be used directly in formulations, or lyophilized, as desired, using well known methods. Lyophilized virus is typically maintained at about 4° C. When ready for use, the lyophilized virus is reconstituted in an appropriate stabilizing solution. Many stabilizing solutions are known. Immunogenic compositions including paramyxovirus of the invention can include a physiologically acceptable carrier and/or adjuvant. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. Lyophilized preparations are generally combined with a sterile solution prior to administration.

The compositions may include pharmaceutically acceptable auxiliary substances as required to approximate physiological conditions, such as pH adjusting and buffering agents, tonicity adjusting agents, wetting agents and the like, such as for example, sodium acetate, sodium lactate, sodium chloride, potassium chloride, calcium chloride, sorbitan monolaurate, triethanolamine oleate, and the like. Acceptable adjuvants include, but are not limited to, Freund's adjuvant (incomplete or complete), MPLTM (3-O-deacylated monophosphoryl lipid A; Corixa, Hamilton Ind.) and IL-12 (Genetics Institute, Cambridge Mass.), CpG oligonucleotides, immunostimulating compositions and alum salts.

The immunogenic compositions of the invention can be administered nasally in droplet, aerosol, or nebulizer form, orally, or parentally, including subcutaneous injection, intravenous, intramuscular, intrasternal or infusion techniques, in dosage unit formulations including conventional non-toxic pharmaceutically acceptable carriers, adjuvants or vehicles. Compositions of the invention can be in the form of suspensions or tablets suitable for oral administration or sterile injectable preparations, such as sterile injectable aqueous or oleagenous suspensions.

For administration as injectable solutions or suspensions, the immunogenic compositions of the invention can be formulated according to techniques well-known in the art, using suitable dispersing or wetting and suspending agents, such as sterile oils, including synthetic mono- or diglycerides, and fatty acids, including oleic acid.

Immunization by the nasal route may be more effective compared with intramuscular or subcutaneous injection because the production of local secretory IgA in the upper respiratory tract can protect against PIV infection. For example, the increased immunity provided by PIV specific secretory IgA can result in a broader cross-reactivity for variant strains of PIV and thus may offer a greater degree of protection against mutant PIV. In contrast, injectable vaccines are inefficient at inducing mucosal IgA. In particular, nasal administration of the immunogenic compositions of the invention may be more effective in the elderly since, unlike the systemic immune system, mucosal immune responses do not deteriorate with age. Immunogenic compositions of the invention that also stimulate systemic immune responses may protect the lower respiratory tract (lungs) due to transudation of antibodies from the serum. In addition, PIV-specific cytotoxic T cells (CTL) in nasal associated lymphoid tissue can contribute to recovery from infection.

Immunogenic compositions for nasal administration are preferably formulated so that they are similar to nasal secretions in regard to toxicity, pH, and viscosity so that normal ciliary action is maintained. In an embodiment, the immunogenic compositions of the invention are formulated in an aqueous solution that is isotonic and slightly buffered to maintain a pH of about seven. Antimicrobial preservatives and appropriate stabilizers, if required, are included in the formulation.

The invention also provides methods of making and using the recombinant paramyxovirus of the invention. In one embodiment, the invention relates to methods of generating an infectious, self-replicating paramyxovirus as have been described herein. The methods generally include the steps of removing or altering a polycistronic polynucleotide encoding a P and C protein from the viral genome or antigenome of a paramyxovirus and inserting a SSRF polynucleotide encoding a P protein and/or a second polynucleotide, which optionally is also a SSRF polynucleotide, encoding at least one C protein. In an alternative embodiment, the variant polynucleotide encoding the P gene in the paramyxovirus can be altered in situ to no longer encode any C protein using the standard methods and the second polynucleotide encoding at least one C protein can be inserted into the paramyxovirus genome or antigenome.

In another embodiment, the methods generally include the steps of removing or altering a polycistronic polynucleotide encoding a P and C protein from the viral genome or antigenome of a paramyxovirus and inserting a SSRF polynucleotide encoding a C protein and/or a SSRF polynucleotide encoding a P protein. In an alternative embodiment, the polynucleotide encoding the C gene in the paramyxovirus can be altered in situ to no longer encode the P protein using the standard methods and the SSRF polynucleotide encoding the P protein can be inserted into the paramyxovirus genome or antigenome.

One or more of the C proteins optionally has at least one mutation that reduces the ability of the C protein to inhibit production of interferon, interferon signaling, or other C functions.

In an embodiment, the removing step includes introducing unique restriction enzyme recognition sequences into the genome or antigenome such that the recognition sequences flank the polycistronic polynucleotide encoding P and C proteins and digesting the genome or antigenome with one or more restriction enzymes that cut the genome or antigenome at the restriction sites flanking the polycistronic polynucleotide. In an embodiment, the inserting step includes inserting the SSRF polynucleotide encoding a P protein and a second polynucleotide encoding at least one C protein at the cleaved restriction sites and relegating the genome or antigenome. In another embodiment, the inserting step includes inserting a SSRF polynucleotide encoding a C protein and a SSRF polynucleotide encoding a P protein at the cleaved restriction sites and relegating the genome or antigenome.

In another embodiment, the invention relates to methods of producing infectious, self-replicating, recombinant paramyxovirus. The methods of the invention include transfecting a population of cells with an expression vector comprising a partial or complete polyhexameric genome or antigenome sequence and one or more supporting vectors including one or more polynucleotides encoding N protein, P protein, and L polymerase, and incubating the transfected cells under conditions to allow for viral replication. The paramyxoviruses have a SSRF polynucleotide encoding a P protein and/or a second polynucleotide encoding at least one C protein.

In another embodiment, the invention relates to methods of eliciting an immune response in a mammal. The methods of the invention include administering an immunogenic composition of the invention. Preferably, the immune response produces antibodies that are protective (e.g. inhibit infection or reduce the severity of infection). In an embodiment, the antibodies are anti-PIV antibodies. In an embodiment, the anti-PIV antibodies are IgA. In some embodiments, the immune response produces antibodies that bind one or more antigenic determinants of a heterologous pathogen encoded by supernumerary genes or genome segments. Examples of heterologous pathogens include but are not limited to, HPIV1 (heterologous to HPIV3), HPIV2, HPIV3 (heterologous to HPIV1), measles virus, subgroup A or subgroup B respiratory syncytial virus, mumps virus, human papilloma virus, type 1 or type 2 human immunodeficiency virus, herpes simplex virus, cytomegalovirus, rabies virus, Epstein Barr virus, filovirus, bunyavirus, flavivirus, alphavirus, human metapneurnovints, or influenza virus. In an embodiment, the antigenic determinants include measles virus HA, HPIV1 HN or F, HPIV2 HN or F, and/or HPIV3 HN or F.

In another embodiment, the invention relates to methods of inhibiting a paramyxovirus infection including, but not limited to, PIV infection, including HPIV1 or HPIV3 infection. The methods of the invention include administering an immunogenic composition of the invention comprising an attenuated paramyxovirus of the invention. The paramyxovirus used in this aspect of the invention includes a SSRF polynucleotide encoding a P protein and a second polynucleotide, which is optionally also a SSRF polynucleotide, encoding at least one C protein. In an embodiment, the paramyxovirus is PIV. In an embodiment, the PIV is HPIV1 or HPIV3. Preferably the immunogenic composition elicits antibodies that are protective (e.g. inhibit infection or reduce the severity of infection). In an embodiment, the antibodies are anti-PIV antibodies. In an embodiment, the anti-PIV antibodies are IgA.

Different embodiments of the invention may display different aspects of the invention. Not every embodiment will exhibit every advantage of the invention.

All publications, patents and patent applications mentioned herein are hereby incorporated by reference in their entirety and for all purposes.

The following examples are provided for illustrative purposes only, and are in no way intended to limit the scope of the present invention.

### Example 1

### Cloning Strategy for the Production of the pFLC-rHPIV1-C+P cDNA Plasmid

Separating the overlapping open reading frames (ORFs) of the P/C gene of HPIV1 into two separately transcribed gene units.

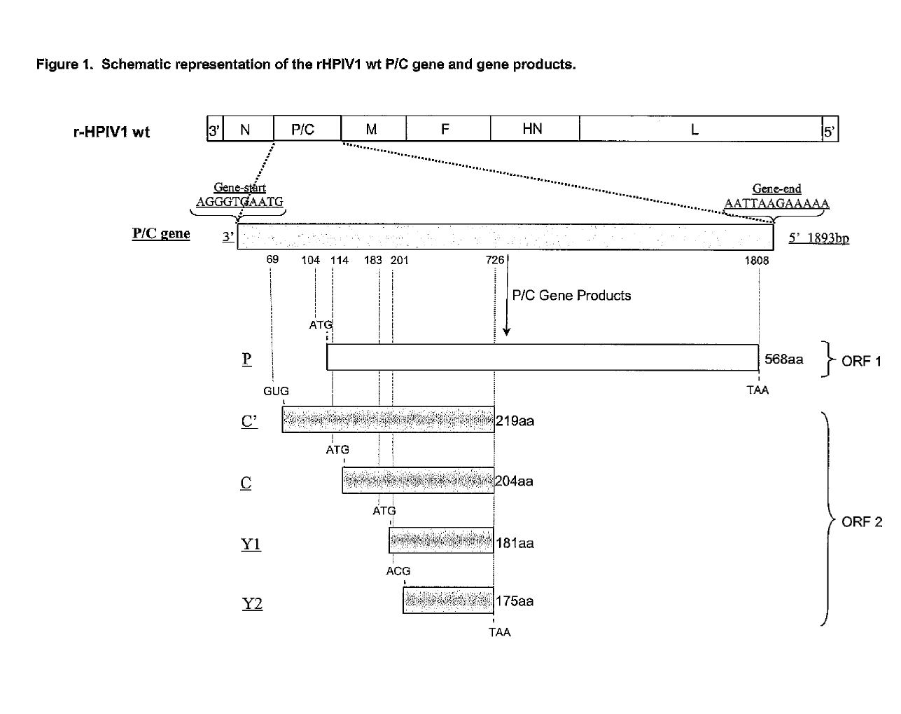

The starting point in constructing pFLC-rHPIV1-C+P was the consensus sequence of the wild type (wt) HPIV1 strain Washington/20993/1964 (Genbank accession # AF457102). A recombinant full-length DNA clone (FLC) of this HPIV1 was made and, using site-directed mutagenesis, 3 additional unique restriction enzyme sites were introduced: MluI (ACGCGT, pre-N ORF position, nucleotide numbers 113-118), AscI (GGCGCGCC, pre-P/C ORF position, nucleotide numbers 1801-1808) and NotI (GCGGCCGC, post-P/C ORF position, nucleotide numbers 3634-3641). In addition this clone contained a single point mutation in the HN gene, HN^{T553A}, which has been previously described [Bartlett, E. J. 2006. Vaccine. 24:2674-2684]. This cDNA clone was termed pFLC-rHPIV1-MluI/AscI/NotI, and was used to prepare pFLC-rHPIV1-C+P. Whereas HPIV1 expresses the P and C proteins from overlapping ORFs in a single mRNA (FIG. 1), in pFLC-rHPIV1-C+P these two overlapping ORFs have been separated into two gene units that express P and C proteins separately from two individual mRNAs (FIG. 2). Standard recombinant DNA methods were used to construct the two new gene units, one designed to express the C accessory proteins (C′, C, Y1 and Y2) but not the P protein, and the other to express the P protein but not the C proteins. The full-length viral cDNA was designed to conform to the rule of six. In the description below, note that all nucleotide (nt) sequence positions are based on the sequence of the fully assembled pFLC-rHPIV1-C+P HN^{T553A} sequence provided in the accompanying sequence listing (SEQ ID NO:2).

### Assembly of the C(P−) Gene Unit

The C gene unit was produced first (Gene Unit 1 of FIG. 2) and was designed to incorporate changes which would enable the C proteins but not the P protein to be expressed from this gene unit. The incorporated changes would not affect the amino acid sequence of the C proteins, i.e., these changes would be silent in the C ORF. In addition, this clone contained a single point mutation in the HN gene, HN T553A, which has been previously described and does not on its own confer a significant att or is phenotype [Bartlett, E. J. et al., Vaccine 24:2674-2684 (2006)]. The cDNA was PCR amplified using primers containing the AscI (3′ end) and NotI (5′ end) restriction enzyme sites on each end to enable cloning into the pFLC-rHPIV1-MluI/AscI/NotI vector. Features incorporated into the C gene unit cDNA included:

1. The PCR was designed to amplify the C ORF and to truncate the P/C gene 25 nt after the C stop codon. It also placed PacI, NheI, and NotI sites in tandem succession on the downstream end. The Pad and NheI sites would later be used to accept gene junction sequence and P gene unit sequence (see Assembly of the P(C−) Gene Unit, below) and in the final pFLC-rHPIV1-C+P HN^{T553A} construct would have the following sequence positions: Pad (nt #2492-2499), NheI (nt #4245-4250).
2. Interruptions to the P open reading frame (ORF) were introduced such that the P protein would not be expressed from the C gene unit. These changes would be silent in the C ORF. This included:
Conversion of the P Start codon (ATG→GTG)
   Nt #1844-1846, pFLC-rHPIV1-C+P HNT553A cDNA (Sequence file #:4).
Stop codons inserted 2 and 5 codons after the P start codon.
   Nt #1850-1852, pFLC-rHPIV1-C+P HNT553A cDNA (Sequence file #:4).
   Nt #1859-1861, pFLC-rHPIV1-C+P HNT553A cDNA (Sequence file #:4).

The above-mentioned truncation of the P/C gene 25 nt after the C stop codon, which removed much of the P ORF.

This C(P−) gene unit was cloned into the pFLC-rHPIV1-HN^{T553A} cDNA at the unique AscI and NotI restriction enzyme sites using standard molecular cloning techniques. The resulting cDNA clone was termed pFLC-rHPIV1-C(P−)HN^{T553A}.

### Assembly of the P(C−) Gene Unit

The P gene unit was produced next (Gene Unit 2 of FIG. 2) and was designed to incorporate changes which would enable the P protein, but not the C proteins, to be expressed from this gene unit, i.e., these changes would be silent in the P ORF. The cDNA was PCR amplified using primers containing the Pad and NheI restriction enzyme sites at each end (3′ and 5′ ends, respectively) to enable cloning into the new pFLC-rHPIV1-C(P−) HN^{T553A}. Features incorporated into the P gene unit cDNA included:

1. The addition at the upstream end of a new gene-end sequence to act as the “C gene end” (sequence same as P gene end sequence); an intergenic region; and a new gene-start sequence to act as the “P gene start” (sequence same as N gene start sequence); these were added directly after the Pad restriction enzyme site.
C Gene End, nt #2494-2505, pFLC-rHPIV1-C+P HNT553A cDNA (See, SEQ ID NO: 2). (note that the Pad site and C Gene End share sequence identity for 6 nt and overlap at nt #2494-2499)
Intergenic sequence, nt #2506-2508, pFLC-rHPIV1-C+P HNT553A cDNA (See, SEQ ID NO:2). P Gene Start, nt #2509-2518, pFLC-rHPIV1-C+P HNT553A cDNA (SEQ ID NO: 2).
2. Truncation of the start of the P/C gene such that only 6 nucleotides lie between the P gene start sequence and the P start codon. These nts are nonviral in origin and have the sequence CCCAAC (nt #2519-2524, pFLC-rHPIV1-C+P HNT553A cDNA (SEQ ID NO:2).
3. Interruptions to the C open reading frame (ORF) such that the C proteins would not be expressed from the P gene unit and that these changes would be silent in the P ORF. This included:

Conversion of the C start (ATG→ACG)

Nt #2535-2537, pFLC-rHPIV1-C+P HNT553A cDNA (SEQ ID NO:2).

Stop codons inserted 1 codon after Y1 start codon and 1 and 10 codons after Y2 start codon. (The Y1 and Y2 start codons were not modified because they could not be converted without affecting the P ORF).

Nt #2607-2609, pFLC-rHPIV1-C+P HNT553A cDNA (SEQ ID NO:2).
Nt #2625-2627, pFLC-rHPIV1-C+P HNT553A cDNA (SEQ ID NO:2).
Nt #2652-2654, pFLC-rHPIV1-C+P HNT553A cDNA (SEQ ID NO:2).

The end of the P/C gene was truncated, leaving 13 nt sequence immediately after the P stop codon, followed by the NheI and NotI sites.

The P(C−) gene unit was cloned into the pFLC-rHPIV1-C(P−) HN^{T553A} cDNA at the PacI and NheI restriction enzyme sites. This generated the pFLC-rHPIV1-C+P HN^{T553A} cDNA.

The entire full-length HPIV1 antigenomic cDNA clone (FLC) was sequenced to confirm that it contained the correct introduced sequences and that no adventitious mutation(s) had been introduced during PCR amplification. (See FIG. 2).

### Example 2

### Production of Variants of rHPIV1-C+P Containing Mutations in the C Gene Unit

A) Introducing Mutations into the pFLC-rHPIV1-C+P cDNA Plasmid:

Modified versions of the pFLC-rHPIV1-C+P HN^{T553A} cDNA clone were prepared by site-directed mutagenesis and standard molecular cloning techniques to introduce mutations of interest into the C proteins of the virus, see Tables 1 and 3. Mutations were inserted into Gene Unit 1 of the pFLC-rHPIV1-C+P HN^{T553A} cDNA (FIG. 2) such that the virus would express variant C proteins and a wild type P protein. When introducing defined mutations into the C gene unit, it is desirable not to have unintended mutations introduced into the P protein since such mutations could affect a function of P that could adversely alter the properties of the recovered virus. Such properties could include an alteration in the growth of such a vaccine candidate, i.e., one bearing an attenuating mutation in C and an unintended mutation in P, in cells used for vaccine manufacture.

The mutations introduced in C included point and deletion mutations affecting codons 83, 84, 85, 169, 170 or 171 of the C protein (Table 3). Note in FIG. 3, that each of these mutations affects the C′, C. Y1, and Y2 proteins. In each case, the region containing the introduced mutation in each FLC was sequenced to ensure that the FLC contained the introduced mutation. It can be seen by examining Table 3 and FIG. 3, that viable recombinant HPIV1 mutants bearing genetically stable deletion mutations in C, but expressing a wild type P protein, are made that are capable of replication in cells in tissue culture (see below).

### B) Deleting Codon 84 in the rHPIV1 Wild Type P/C Backbone has a Greater Negative Effect On Replication In Vitro than does Such a Deletion in the rHPIV1-C+P Backbone

Viruses containing deletions at codon 84 in the C protein (C^{Δ83-84} and C^{Δ84-85}) in both the rHPIV1 wt and rHPIV1-C+P backbones were recovered and designated rHPIV1-P/C^{Δ83-84}HN^{T553A}, rHPIV1-P/C^{Δ84-85}HN^{T553A}, rHPIV1-C^{Δ83-84}+P HN^{T553A}, or rHPIV1-C^{Δ84-85}+P HN^{T553A} (Groups 2 and 3, Table 3). The kinetics of replication for these viruses was examined in a multi-cycle growth curve in LLC-MK2 cells (multiplicity of infection=0.01) compared to the corresponding rHPIV1 wt and rHPIV1-C+P viruses (FIG. 4). The viruses included in this growth curve were from uncloned virus suspensions, and this data has been confirmed with biologically cloned virus for select clones.

The rHPIV1-C+P grew to similar titers as rHPIV1 wt with peak titers reaching 9.0 and 8.7 log₁₀TCID₅₀/ml, respectively. This indicates that the separation of the two genes in rHPIV1-C+P results in a virus that can replicate as well as wild type rHPIV1. The rHPIV1-P/C^{Δ83-84}HN^{T553A} and rHPIV1-P/C^{Δ84-85}HN^{T553A} viruses in which both the C and P ORFs are affected by the deletions were moderately attenuated for replication in LLC-MK2 cells in vitro with peak titers reaching 7.2 and 6.9 log₁₀TCID₅₀/ml, respectively. The rHPIV1-C^{Δ83-84}+P HN^{T553A} and rHPIV1-C^{Δ84-85}+P HN^{T553A} viruses in which the deletion affects only the C proteins, but not P, both grew to 7.9 log₁₀TCID₅₀/ml, i.e., about 8-10-fold higher. Deletion of codons in both the C and P proteins specifies a stronger attenuating effect on replication in vitro than deleting codons in C only. This is probably due to a deleterious effect of the mutation in the P protein on virus replication for both the rHPIV1-P/C^{Δ83-84}HN^{T553A} and rHPIV1-P/C^{Δ84-85}HN^{T553A} viruses, i.e., viruses that contained desired mutations in C but unwanted mutations in P. The 10-fold increase of replication of this virus rHPIV1-C^{Δ83-84}P HN^{T553A} and rHPIV1-C^{Δ84-85}+P HN^{T553A} versus the rHPIV1-P/C^{Δ83-84}HN^{T553A} and rHPIV1-P/C^{Δ84-85}HN^{T553A} viruses would be advantageous during manufacture of the former viruses.

### Example 3

### Cloning Strategy for the Production of HPIV3-P+C.

The starting point for constructing HPIV3-P+C was pUC(GE/GS)_{P-M} (Skiadopoulos et al Virology 297:136-152, 2002). This was a PmeI-BamHI fragment spanning nt 1215-3903 of the HPIV3 wt genome that had been subcloned and modified as follows: (i) individual nucleotides within positions 3693-3698 (of the complete antigenome sequence) were changed to introduce an AflI site, and (ii) this AflI site was used to accept a short DNA insert that contained an HPIV3 gene junction followed by several convenient cloning sites (Skiadopoulos et al Virology 297:136-152, 2002). The resulting plasmid pUC(GE/GS)_{P-M} was thus designed so that an insert cloned into a MluI site would be situated downstream of the P gene and under the control of an independent set of gene-start and gene-end signals.

As shown in FIG. 5A, the P/C/V/D gene of HPIV3 has the potential to encode a complex array of proteins. P and C are expressed separately from overlapping ORFs. In addition, there are downstream D and V ORFs. D is accessed by RNA editing, and its synthesis is directly linked to that of the P gene (for general reference, use the Collins and Karron 2006 Fields Chapter). The mechanism of expression of V is unknown, although there is genetic evidence that it is indeed expressed. In most other paramyxoviruses, V is accessed by RNA editing, and its synthesis is directly linked to that of P. In any event, both D and V are located downstream of C and their expression is thought to be independent of C. Thus, in HPIV3-P+C, the P and C ORFs are segregated into separate polynucleotides, and D and V co-segregate with P (FIG. 5B).

The first step is to engineer a copy of the P/C/V/D gene to make a polynucleotide in which expression of C was silenced (FIG. 5C). This involved using standard methods of site-directed mutagenesis to introduce point mutations into pUC(GE/GS)_{P-M} that (i) ablate the start codon of the C ORF, and (iii) introduce three translational start codons into the C ORF (FIG. 5C). Each of these changes would be silent with regard to the overlapping P ORF (as well as the V and D ORFs, of course, since these are downstream and do not overlap C).

The second step is to engineer a second copy of the P/C/V/D gene to make a second polynucleotide in which expression of P is silenced and in which V and D are deleted. This involves PCR using primers that introduce a MluI site on either side of the ORF and, in addition, delete the start site of the P ORF as well as the downstream half of the P/C/V/D gene (FIGS. 5D and 5E). The resulting Mlul fragment is cloned into the MluI site of pUC(GE/GS)_{P-M}. The resulting plasmid would then be subjected to site-directed mutagenesis using conventional methods to introduce two point mutations that create two translational stop codons in the P ORF without affecting the C ORF, and which change the second ATG in the P ORF to CTG without affecting the C ORF (FIG. 5D).

The resulting modified version of pUC(GE/GS)_{P-M}, designated pUC(GE/GS)_{P-M}(P+C). would be subjected to restriction digestion with PmlI and BamHI, and the resulting fragment cloned into the corresponding window of the complete antigenomic cDNA.

TABLE 2
Summary of the preferred L gene mutations for introduction
into rHPIV1-C + P.

| | | | | Codon | | # nt changes for |
|---|---|---|---|---|---|---|
| | | nt changes | Type of | position | Amino acid | reversion to wt |
| Mutation^{a} | ORF | wt→mutant^{b} | mutation | in L | change | amino acid |
| 1 Δ1710- | L | GCT GAG → | Deletion | 1710-11 | AE deletion | 6 (insertions) |
| 11^{c} | | →deletion | | | | |
| 2 Y942A^{d} | L | TAT → GCG | Point | 942 | Y → A | 3e |
| 3 F456L | L | TTT → CTG | Point | 456 | F → L | 2 |
| 4 Y942T^{d} | L | TAT → ACA | Point | 942 | Y → T | 3 |
| 5 L992C^{d} | L | TTA → TGC | Point | 992 | L → C | 2 |
| 6 L992N^{d} | L | CTC → AAC | Point | 992 | L → N | 2 |

^{a}The nomenclature used to describe each mutation indicates the wt amino acid, the codon position and the new amino acid, or the position of the deletion (Δ), with respect to the C, HN or L protein.
^{b}The nucleotides (nt) affected by substitution or deletion are shown underlined and in bold type.
^{c}Designed for increased genetic stability by use of a deletion. Deletions involved six nt to conform to the rule of six.
^{d}Designed for increased genetic stability by the use of a codon that differs by three nucleotides from codons yielding a wild type assignment.

TABLE 3
Summary of the C and P mutations introduced into the rHPIV1 genome.

| | | | | | | | Re- | | |
|---|---|---|---|---|---|---|---|---|---|
| | | | | | | | covered | | |
| Mutations | | | Type | nt # | | | as In- | Attenuated for | |
| of | | nt changes | of | (viral | Affected | Affected | fectious | replication? | |
| Interest^{a} | Backbone | wt→mutant^{b} | mutation | genome)^{c} | Gene | ORFs | Virus? | In Vitro | In Vivo |
| 1 R84G | pFLC- | A→G | point | 2103 | P/C | C′C/Y1/Y2 | yes | no | yes (Hamsters |
| | rHPIV1 | | | | | and P | | | only) [also |
| | wt | | | | | | | | yes as a set |
| | | | | | | | | | with HN^{T553A} |
| | | | | | | | | | only in |
| | | | | | | | | | African Green |
| | | | | | | | | | Monkeys] |
| 2 Δ83-84 | pFLC- | AGG AGA | deletion | 2100-2105 | P/C | C′/C/Y1/Y2 | yes | yes | yes (Hamsters |
| | rHPIV1 | | | | | and P | | (to be | only) |
| | wt | | | | | | | con- | |
| | | | | | | | | firmed) | |
| | pFLC- | | | 2100-2105 | C | C′/C/Y1/Y2 | yes | no | yes (Hamsters |
| | rHPIV1- | | | | | | | (to be | only) |
| | C + P^{d} | | | | | | | con- | |
| | | | | | | | | firmed) | |
| 3 Δ84-85 | pFLC- | AGA GTG | deletion | 2103-2108 | P/C | C′/C/Y1/Y2 | yes | yes | Not done |
| | rHPIV1 | | | | | and P | | | |
| | wt | | | | | | | | |
| | pFLC- | | | 2103-2108 | C | C′/C/Y1/Y2 | yes | no | yes |
| | rHPIV1- | | | | | | | | (Hamsters and |
| | C + P^{d} | | | | | | | | African Green |
| | | | | | | | | | Monkeys) |
| 4 F170S | pFLC- | T→C | point | 2362 | P/C | C′/C/Y1/Y2 | yes | no | yes |
| | rHPIV1 | | | | | | | | (Hamsters and |
| | wt | | | | | | | | African Green |
| | | | | | | | | | Monkeys) |
| 5 Δ170 | pFLC- | GGA TTT | deletion | 2357-2362 | P/C | C′/C/Y1/Y2 | yes | no | yes |
| | rHPIV1 | | | | | and P | | | (Hamsters and |
| | wt | | | | | | | | African Green |
| | | | | | | | | | Monkeys) |
| 6 Δ169- | pFLC- | GAT TTC | deletion | 2358-2363 | C | C′/C/Y1/Y2 | yes | Not done | yes |
| 170 | rHPIV1- | | | | | | | | (Moderate in |
| | C + P^{d} | | | | | | | | African Green |
| | | | | | | | | | Monkeys) |
| 7 Δ170- | pFLC- | TTC CAG | deletion | 2361-2366 | C | C′/C/Y1/Y2 | yes | Not done | Not done |
| 171 | rHPIV1- | | | | | | | | |
| | C + P^{d} | | | | | | | | |

^{a}The nomenclature used to describe each mutation within the genome contains the wt amino acid, the codon position and the new amino acid or deletion (Δ) with respect to the C protein.
^{b}The altered nucleotides (nt) are shown underlined and in bold type.
^{c}The nucleotide numbers refer to the nucleotide position in the viral genome of the indicated rHPIV1 backbone.
^{d}This backbone contains the HN^{T553A} mutation.

### Example 4

### Kinetics of Replication of rHPIV-P(C−) in Culture

The rHPIV1 wt and mutant viruses were compared in multicycle growth curves. Confluent monolayer cultures of LLC-MK2 and Vero cells in 6-well plates were infected in triplicate at a MOI of 0.01 TCID₅₀/cell. Virus adsorption was performed for 1 h in media containing trypsin. The inoculum was then removed and cells were washed three times, after which fresh medium containing trypsin was added and then harvested as the day 0 sample and replaced with fresh media containing trypsin. On days 1-7 p.i., the entire supernatant was removed for virus quantitation and was replaced with fresh medium containing trypsin. Supernatants containing virus were frozen at −80° C., and virus titers (log₁₀ TCID₅₀/ml) were determined with endpoints identified by hemadsorption. Cytopathic effect (cpe) was visually monitored. The amount of cpe observed under the microscope was given a score ranging from 1-5 based on the percentage of cells in the monolayer showing cpe. Cpe of less than 20% of cells was scored as 1; 21-40% as 2; 41%-60% as 3; 61-80% as 4; 81-100% as 5. Results are shown in Table 4.

TABLE 4
Cytopathicity of HPIV mutants

| | | CPE Scores | | | | | | | | | | | | | |
|---|---|---|---|---|---|---|---|---|---|---|---|---|---|---|---|
| | | MK2 Cells | | | | | | | | | | | | | |
| | | (Days post-infection) | | | | | | | Vero Cells (Days post-infection) | | | | | | |
| Virus | | 1 | 2 | 3 | 4 | 5 | 6 | 7 | 1 | 2 | 3 | 4 | 5 | 6 | 7 |
| 1 | rHPIV1-HN^{T553A} | 1 | 1 | 1 | 1 | 1 | 1 | 1 | 1 | 1 | 1 | 1 | 1 | 1 | 1.7 |
| 2 | rHPIV1-C + P HN^{T553A} | 1 | 1 | 1 | 2.5 | 2.7 | 3.5 | 4 | 1 | 1 | 1 | 1 | 1 | 1.3 | 2 |
| 3 | rHPIV1-P/C^{Δ84-85} HN^{T553A} | 1 | 1 | 1 | 1.8 | 2.7 | 3.3 | 4.7 | 1 | 1 | 1 | 1 | 1 | 1.2 | 2 |
| 4 | rHPIV1-C^{Δ84-85} + P | 1 | 1 | 2.2 | 4 | 4.5 | 5 | 5 | 1 | 1 | 1 | 1.5 | 2 | 3.7 | 4.7 |
| | HN^{T553A} | | | | | | | | | | | | | | |

Separating the P and C open reading frames into two separate gene units offers a distinct advantage for in vitro replication of rHPIV1s with mutations in the C proteins. This is shown in multicycle growth curves comparing the replication of mutants containing the Δ84-85 mutation on the wt backbone vs the C+P backbone, i.e. rHPIV1-P/C^{Δ84-85} and rHPIV1-C⁸⁴⁻⁸⁵+P, respectively (FIG. 6). These data clearly demonstrate a replication advantage for mutants containing the Δ84-85 deletion mutation on the C+P backbone. rHPIV1-C^{Δ84-85}+P reached peaks of 8.2 and 6.7 log₁₀ TCID₅₀/ml in LLC-MK2 and Vero cells respectively, compared to rHPIV1-P/C^{Δ84-85} at 6.5 and 5.2 log₁₀ TCID₅₀/ml. Clinical lots of the viruses will be prepared in Vero cells and a titer of approximately 7 is required for efficient manufacturing purposes. Therefore, it is evident that separating the P and C genes in viruses to be used for HPIV vaccines is advantageous in respect to providing high titer for manufacturing. The ability to introduce mutations into the C gene without affecting P can greatly improve the in vitro replication competence of viruses to be used for vaccine purposes.

### Example 5

### The Effect of Infection with rHPIV1 Wt or Mutants on Type I IFN Signaling

The ability of the rHPIV1s to inhibit type I IFN signaling was determined by their ability to block the establishment of an antiviral state by IFN, as indicated by the level of VSV replication following IFN treatment. Vero cells in 6-well plates were infected with the indicated rHPIV1s at a MOI of 5 TCID₅₀/cell and incubated for 24 h. Cells were then left untreated or were treated with 100 or 1000 IU/ml IFN-β (AVONEX; Biogen, Inc., Cambridge, Mass.) (1 well per treatment per virus) for 24 h. The cells were then infected with VSV-GFP, under a 0.8% methyl cellulose overlay, and incubated for 48 h. The VSV-GFP foci were visualized using a typhoon 8600 phosphorimager (Molecular Dynamics, Sunnyvale, Calif.) and counted. FIG. 7 presents the percent inhibition of VSV-GFP replication in IFN-β treated versus untreated cells based on two independent experiments.

### Example 6

### Efficacy of rHPIV1 wt and Mutants Against Challenge with Wild-Type HPIV1

Replication and induction of a protective immune response after immunization with wild-type and various mutant HPIV1 were assessed in Syrian Golden hamsters and in African Green Monkeys (Tables 5-7, FIG. 8).

Inoculation of hamsters with rHPIV1 mutants containing deletions at codon 84 either on the wild type or the C+P backbone, demonstrated attenuation of replication in both the upper respiratory tract (URT) and lower respiratory tract (LRT), regardless of the backbone the deletion was in. In contrast, inoculation of hamsters with a rHPIV1 containing a single point mutation at codon 84 demonstrated replication at wt level in the URT and a moderate reduction of replication in the LRT. Therefore, the use of a deletion mutation at codon 84 offers advantages over the use of a point mutation at this codon since it is not only more stable but also more attenuating in hamsters. Protective efficacy against HPIV1 wt challenge was determined next in hamsters that had been inoculated with these viruses. Although rHPIV1-C^{Δ83-84}-+P HN^{T553A}, rHPIV1-C^{Δ84-85}+P HN^{T553A} and rHPIV1-C/P^{Δ83-84}HN^{T553A} all grew to similar levels following initial inoculation, only rHPIV1-C^{Δ84-85}+P HN^{T553A} protected against challenge with HPIV1 wt. Since rHPIV1-C^{Δ84-85}+P HN^{T553A} replicated efficiently in vitro and was attenuated and efficacious in hamsters, we proceeded to studies in African green monkeys with this virus.

AGMs (n≧4) were inoculated i.n. and intratracheally (i.t.) with 10⁶ TCID₅₀ of either HPIV1 wt or mutant rHPIV1 in a 1 ml inoculum at each site. Nasopharyngeal (NP) swab samples were collected on days 0 to 10 post-infection and tracheal lavage (TL) fluid samples were collected on days 2, 4, 6, 8, and 10 post-infection. On day 28 p.i., the AGMs were challenged i.n. and i.t. with 10⁶ TCID₅₀ of HPIV1 wt in 1 ml L-15 per site. NP swab samples and TL fluid samples were collected for virus quantitation on days 0, 2, 4, 6 and 8 and days 2, 4, 6 and 8 post-challenge, respectively. The specimens were flash frozen and stored at −80° C. until they were assayed in parallel. Virus present in the samples was titered in serial dilutions on LLC-MK2 cell monolayers in 96-well plates, and an undiluted 100 μl aliquot also was tested in 24-well plates. Following incubation for 7 days, virus was detected by hemadsorption, and the mean log₁₀ TCID₅₀/ml titer was calculated for each sample day. The limit of detection was 0.5 log₁₀ TCID₅₀/ml. The mean daily titer for each group is represented on the graph. All animal studies were performed under protocols approved by the National Institute of Allergy and Infectious Disease (NIAID) Animal Care and Use Committee (ACUC).

TABLE 5
Replication of rHPIV1 mutants in the upper and lower respiratory tract of hamsters.

| | | Number | | | | |
|---|---|---|---|---|---|---|
| | | of | Nasal turbinates^{b} | | Lungs^{b} | |
| Virus^{a} | | animals | Day 4^{c} | Day 5^{c} | Day 4^{c} | Day 5^{c} |
| 1 | rHPIV1-HN^{T553A} | 11 | 5.2 ± 0.2 | 5.3 ± 0.4 | 4.6 ± 0.5 | 4.6 ± 0.4 |
| 2 | rHPIV1-C + P-HN^{T553A} | 10 | 4.1 ± 0.3 | 4.8 ± 0.3 | 2.6 ± 0.3 | 3.1 ± 0.2 |
| 3 | rHPIV1-C^{R84G}HN^{T553A} | 6 | 5.2 ± 0.2 | 4.6 ± 0.2 | 2.8 ± 0.3 | 3.5 ± 0.4 |
| 4 | rHPIV1-C^{Δ83-84} + P-HN^{T553A} | 10 | 2.0 ± 0.2 | 1.9 ± 0.1 | 2.2 ± 0.1 | 2.3 ± 0.1 |
| 5 | rHPIV1-C^{Δ84-85} + P-HN^{T553A} | 10 | 2.4 ± 0.1 | 2.2 ± 0.2 | 2.4 ± 0.2 | 2.3 ± 0.2 |
| 6 | rHPIV1-C/P^{Δ83-84}-HN^{T553A} | 5 | 1.6 ± 0.1 | 1.7 ± 0.2 | 1.7 ± 0.2 | 2.0 ± 0.2 |

^{a}Hamsters in groups of 5 or 6 were inoculated i.n. with 10^{5.5} TCID₅₀ of the indicated virus. The data is representative of two independent experiments. rHPIV1-HN^{T553A} has been shown to replicate to wt-like effect in vivo [Bartlett, E. J. et al., Vaccine 24: 2674-2684 (2006)].
^{b}Virus present in the tissues was quantified by serial dilution on LLC-MK2 monolayer cultures at 32° C. and is expressed as the mean virus titer for each group, log₁₀ TCID₅₀/g ± SE. The limit of detection was 1.5 log₁₀ TCID₅₀/g.
^{c}Nasal turbinates and lungs from each group were harvested on day 4 and 5 post-infection.
^{d}Underlined values indicate a 100-fold reduction in virus replication compared to rHPIV1-HN^{T553A}.

TABLE 6
Protection against HPIV1 wt challenge in hamsters following
immunization with rHPIV1 mutants.

| | | | Mean HPIV1 wt titer | |
|---|---|---|---|---|
| | | | (log₁₀ TCID₅₀/g)^{b} | |
| | | | Nasal | |
| Virus^{a} | | No. animals | turbinates^{c} | Lungs^{c} |
| 1 | rHPIV1-HN^{T553A} | 5 | 1.5 ± 0.0 | 1.7 ± 0.2 |
| 2 | rHPIV1-C^{R84G} HN^{T553A} | 5 | 1.7 ± 0.2 | 1.7 ± 0.1 |
| 3 | rHPIV1-C + P HN^{T553A} | 5 | 1.5 ± 0.0 | 1.6 ± 0.1 |
| 4 | rHPIV1-C^{Δ83-84} + P HN^{T553A} | 5 | 2.8 ± 0.5 | 3.0 ± 0.4 |
| 5 | rHPIV1-C^{Δ84-85} + P HN^{T553A} | 5 | 1.9 ± 0.2 | 2.1 ± 0.2 |
| 6 | rHPIV1-C/P^{Δ83-84} HN^{T553A} | 5 | 4.4 ± 0.4 | 2.7 ± 0.2 |
| 7 | Non-immunized | 5 | 4.5 ± 0.2 | 3.9 ± 0.4 |

^{a}Hamsters in groups of 5 or 6 were inoculated i.n. with 10⁶ TCID₅₀ of the indicated virus. rHPIV1-HN^{T553A} has been shown to replicate to wt-like effect in vivo [Bartlett, E. J. et al., Vaccine 24: 2674-2684 (2006)].
^{b}Virus present in the tissues was quantified by serial dilution on LLC-MK2 monolayer cultures at 32° C. and is expressed as the mean of virus titer, log₁₀ TCID₅₀/g ± SE. The limit of detection was 1.5 log₁₀ TCID₅₀/g.
^{c}Nasal turbinates and lungs from each group were harvested on day 4 post-infection.
^{d}Underlined values underlined indicate a 100-fold reduction in virus replication compared to the non-immunized control group.

TABLE 7
Replication of rHPIV1 mutants in AGMs.

| | | | Mean peak | | Mean sum of the daily | |
|---|---|---|---|---|---|---|
| | | | virus titer | | virus titers (log₁₀ | |
| | | No. | (log₁₀ TCID₅₀/ml)^{c} | | TCID₅₀/ml)^{d} | |
| Virus^{a} | | animals | NP swab | TL | NP swab | TL |
| 1 | HPIV1 wt | 18 | 4.3 ± 0.1 | 4.1 ± 0.3 | 27.4 ± 1.4 | 13.6 ± 1.4 |
| 2 | rHPIV1-HN^{T553A} | 4 | 4.3 ± 0.2 | 4.6 ± 0.2 | 24.9 ± 0.7 | 13.7 ± 1.8 |
| 3 | rHPIV1-C + P HN^{T553A} | 6 | 3.4 ± 0.4 | 4.7 ± 0.4 | 22.3 ± 2.3 | 15.3 ± 1.5 |
| 4 | rHPIV1-C^{R84G} | 12 | 2.1 ± 0.2 | 4.8 ± 0.3 | 10.5 ± 0.9 | 14.3 ± 1.1 |
| | HN^{T553A} | | | | | |
| 5 | rHPIV1-C^{Δ84-85} + P | 4 | 1.6 ± 0.3 | 1.7 ± 0.1 | 7.9 ± 1.3 | 7.8 ± 0.2 |
| 6 | rHPIV1-C^{Δ84-85} + P | 4 | 1.7 ± 0.2 | 1.8 ± 0.3 | 9.4 ± 0.9 | 7.1 ± 0.2 |
| | HN^{T553A} | | | | | |
| 7 | rHPIV1-C^{F170S} HN^{T553A} | 4 | 3.2 ± 0.9 | 2.7 ± 0.9 | 15.4 ± 3.6 | 6.2 ± 1.3 |
| 8 | rHPIV1-C^{Δ170} | 6 | 3.4 ± 0.5 | 2.3 ± 0.5 | 14.8 ± 1.9 | 5.1 ± 0.8 |
| 9 | rHPIV1-C^{Δ169-170} + | 4 | 3.4 ± 0.4 | 3.7 ± 0.3 | 19.4 ± 1.2 | 10.2 ± 1.4 |
| | P HN^{T533A} | | | | | |

^{a}Monkeys were inoculated i.n. and i.t. with 10⁶ TCID₅₀ of the indicated virus in a 1 ml inoculum at each site. Monkeys lacked HAI antibody to HPIV1 wt at the start of the study.
^{b}NP samples were collected on days 0 to 10 post-infection. The titers on day 0 were ≦0.5 log₁₀ TCID₅₀/ml.
^{c}TL samples were collected on days 2, 4, 6, 8, and 10 post-infection. The limit of detection was 0.5 log₁₀ TCID₅₀/ml.
^{d}Mean of the peak virus titers ± SE, irrespective of day, for the animals in each group. Virus titrations were performed on LLC-MK2 cells at 32° C. The limit of detection was 0.5 log₁₀ TCID₅₀/ml.
^{e}Reduction in mean peak virus titer versus HPIV1 wt is expressed as log₁₀ TCID₅₀/g.
^{f}The daily titers for all sampling days were summed for each animal and the mean was determined, shown ± SE.
This indicates the total load of shed virus. The limit of detection for NP samples was 5.5 log₁₀ TCID₅₀/ml and for TL samples was 2.5 log₁₀ TCID₅₀/ml.

Examples 5 and 6 show that C+P mutants containing the Δ84-85 and Δ169-170 demonstrated no inhibition of type I IFN signaling (FIG. 5), a characteristic that has previously been associated with attenuation in vivo (Van Cleve, W., 2006). Indeed, C+P mutants containing these deletions were attenuated in animal models including Syrian Golden hamsters and African Green monkeys (AGMs) (Tables 5 and 7). rHPIV1-C^{Δ84-85}+P was highly attenuated in both AGMs and hamsters and rHPIV1-C^{Δ169-170}+P, demonstrated a moderate level of attenuation in AGMs (Table 7). These mutants provided varying levels of protection in Syrian Golden hamsters against challenge with HPIV1 wt (Table 6 and FIG. 8).

1. An infectious self-replicating parainfluenza virus (PIV) comprising a partial or complete PIV1 or PIV3 genome or antigenome comprising
a) a SSRF polynucleotide encoding a P protein having at least 85% identity to SEQ ID NO:11 or to SEQ ID NO: 22; and
b) a second SSRF polynucleotide encoding a C protein having at least 85% identity to SEQ ID NO:17 or to SEQ ID NO: 23.

2. An infectious self-replicating parainfluenza virus (PIV) comprising a partial or complete PIV1 or PIV3 genome or antigenome comprising
a) a substantial open reading frame (SSRF) polynucleotide encoding a P protein having at least 85% identity to SEQ ID NO:11 or to SEQ ID NO: 22 and from which expression of C, C′, Y1 and Y2 proteins has been ablated or greatly reduced; and
b) a second polynucleotide encoding a C protein having at least 85% identity to SEQ ID NO:11 or to SEQ ID NO: 22 having at least one nucleotide change that ablates or reduces expression of said C protein or that expresses a truncated C protein or that expresses a C protein with reduced activity compared to a native C protein, and from which expression of a P protein has been ablated or greatly reduced.

3. The PIV according to claim 2, wherein said second polynucleotide encodes C proteins having a C-terminal deletion of 14 amino acids.

4. The PIV according to claim 2, wherein said at least one nucleotide change in said polynucleotide encoding the C protein reduces expression of the C protein.

5. The PIV according to claim 2, wherein said at least one nucleotide change in said polynucleotide encoding the C protein reduces activity of the expressed C protein compared to the native C protein.

6. The PIV according to claim 2, wherein said at least one nucleotide change in the polynucleotide encoding the C protein is a member of the group consisting of C^{Δ83-84}, C^{Δ84-85}, C^{Δ169-170}, C^{Δ170-171} and C^{Δ84/Δ170}.

7. The PIV according to claim 1 or 2, further comprising a polynucleotide sequence encoding an HN protein having at least 85% identity to SEQ ID NO:18 or SEQ ID NO: 24 or an L protein having at least 85% identity to SEQ ID NO:13 or SEQ ID NO: 25.

8. The PIV according to claim 7, wherein said FIN polynucleotide has at least one nucleotide change that generates HN^{T553A}.

9-11. (canceled)

12. The PIV according to claim 2, wherein said at least one nucleotide change in the polynucleotide encoding the C protein includes a deletion of amino acids corresponding to amino acids 83-84 (Δ83-84) in SEQ ID NO:17.

13. The PIV according to claim 2, wherein said at least one nucleotide change in the polynucleotide encoding the C protein includes a deletion of amino acids corresponding to amino acids 84-85 (Δ84-85) in SEQ ID NO:17.

14. The PIV according to claim 2, wherein said at least one nucleotide change in the polynucleotide encoding the C protein includes a deletion of amino acids corresponding to amino acids 169-170 (Δ169-170) or a deletion of amino acids corresponding to amino acids 170-171 (Δ170-171) of SEQ ID NO:17.

15-16. (canceled)

17. The PIV according to claim 2, wherein said at least one nucleotide change in the polynucleotide encoding the C protein occurs at one or more of positions corresponding to positions 1854, 1855 and 1856 in SEQ ID NO:1.

18. The PIV according to claim 2, wherein said at least one nucleotide change in said polynucleotide encoding the C protein introduces at least one stop codon that truncates the C protein.

19. The PIV according to claim 7, wherein said L protein has at least one amino acid corresponding to position 456, 942, 992, 1710 or 1711 deleted.

20. The PIV according to claim 1 or 2 comprising a partial or complete HPIV1 or HPIV3 genome or antigenome, further comprising a supernumerary polynucleotide sequence.

21-28. (canceled)

29. A method of immunizing a subject against infection by a pathogen in a subject having or identified as being at risk of developing said infection comprising administering an effective amount of a composition of the PIV according to claim 1 or 2 to said subject.

30-33. (canceled)

34. A method for producing an infectious self-replicating parainfluenza virus (PIV) comprising culturing cells infected with a PIV comprising a partial or complete PIV1 or PIV3 genome or antigenome comprising
a) a SSRF polynucleotide encoding a P protein having at least 85% identity to SEQ ID NO:11 or to SEQ ID NO: 22; and
b) a second polynucleotide encoding a C protein having at least 85% identity to SEQ ID NO:11 or to SEQ ID NO: 22 and having at least one nucleotide change that ablates or reduces expression of said C protein or that expresses a truncated C protein or that expresses a C protein with reduced activity compared to a native C protein; and from which expression of a P protein has been ablated or greatly reduced and
recovering an infectious self-replicating PIV.

35. The method according to claim 34, in which the PIV genome or antigenome further comprises a polynucleotide encoding an FIN protein having at least 85% identity to SEQ ID NO:18 or to SEQ ID NO: 24, wherein said polynucleotide encoding an FIN protein has an HN^{T553A} mutation.

36. The method according to claim 34 or 35, wherein said at least one nucleotide change in the polynucleotide encoding the C protein reduces expression of the C protein.

37. (canceled)

38. The method according to claim 34 or 35, wherein said at least one nucleotide change in the polynucleotide encoding the C protein encodes a mutation selected from the group consisting of C^{Δ84-85}, C^{Δ169-170}, C^{Δ170-171} and C^{Δ84/Δ170}.

39. (canceled)

40. The method according to claim 34, wherein said isolated PIV1 contains both the P and C genes.

41. The method according to claim 34 or 35, wherein the PIV genome or antigenome further comprises at least one supernumerary polynucleotide.

42. (canceled)

43. An isolated polynucleotide comprising
(a) a SSRF polynucleotide encoding a P protein having at least 85% identity to SEQ ID NO:11 or to SEQ ID NO:22;
(b) a SSRF polynucleotide encoding a C protein having at least 85% identity to SEQ ID NO:11 or to SEQ ID NO:22; or
(c) a polynucleotide encoding a C protein having at least 85% identity to SEQ ID NO:11 or to SEQ ID NO: 22 and having at least one nucleotide change that ablates or reduces expression of said C protein or that expresses a truncated C protein or that expresses a C protein with reduced activity compared to a native C protein and from which expression of a P protein has been ablated or greatly reduced.

44. The isolated polynucleotide according to claim 43, wherein said polynucleotide comprises a C protein.

45. The isolated polynucleotide according to claim 44, wherein said at least one nucleotide change is a C-terminal deletion of 14 amino acids.

46. The isolated polynucleotide according to claim 44, wherein said at least one nucleotide change is a change that occurs at one or more of positions corresponding to positions 1854, 1855 and 1856 in SEQ ID NO:1 or is a deletion of amino acids corresponding to amino acids 83-84 (Δ83-84), amino acids 84-85 (Δ84-85), amino acids 169-170 (Δ169-170) or amino acids 170-171 (Δ170-171) of SEQ ID NO:11.

47. An isolated polynucleotide comprising:
a) a SSRF polynucleotide encoding a P protein having at least 85% identity to SEQ ID NO:11 or to SEQ ID NO: 22; and
c) a second SSRF polynucleotide encoding a C protein having at least 85% identity to SEQ ID NO:11 or to SEQ ID NO: 22.

48. An isolated polynucleotide comprising a polynucleotide encoding a partial or complete PIV genome or antigenome comprising the isolated polynucleotide of claim 43 or of claim 47.

49-50. (canceled)

51. The infectious, self-replicating virus of claim 2 that is selected from the group consisting of rPIV1-C^{Δ83-84}+P/HN^{T553A}, rPIV1-C^{Δ84-85}+P/HN^{T553A} and rPIV1-C^{Δ169-170}+P/HN^{T553A}.

52. An isolated polynucleotide comprising a polynucleotide that encodes the genome or antigenome of an infectious, self-replicating virus that is selected from the group consisting of rPIV1-C^{Δ83-84}+P/HN^{T553A}, rPIV1-C^{Δ84-85}+P/HN^{T553A} and rPIV1-C^{Δ169-170}+P/HN^{T553A}.

53. An isolated polynucleotide comprising a polynucleotide that encodes the genome or antigenome of an infectious, self-replicating virus according to claim 3.

54-55. (canceled)


SEQUENCE LISTING (58 sequences)

SEQ ID NO: 1 - DNA, 15600 bp, Human parainfluenza virus
accaaacaagaggaaaaacttgtttggaatatataataatattaaatagtattttagggttaaagtaatactttaaagggacaagtcacagacatttgatcttagtataaatttttataatggccgggctactaagtacttttgacacatttagttccaggagaagtgagagcatcaataagtctggcggaggagcaattatacctggtcaaagaagtaccgtttctgtcttcacattaggcccgagtgtgacagatgatgcagataaattattaatagcaaccactttcttagcccactcattggacacagataaacaacactctcaaagaggaggatttttagtatcactccttgcaatggcctacagtagtccggagttatatctcactacaaacggtgtcaatgctgatgtcaagtatgtgatatacaatatagagagagatcctaaaagaacaaaaacagatgggttcattgtcaaaacaagagacatggagtatgaaagaacaacagagtggttgtttggacctatgatcaacaagaacccattgttccaagggcaaagagagaatgcggatctagaggcattgcttcagacatatggatatcctgcatgtctcggagctataatagttcaagtttggatagtgttggttaaagccataacaagtagtgctggtctaagaaaaggattcttcaatagattagaagcattcagacaggatggaaccgttaaaagtgccctggtcttcacaggagacacagttgaaggtattggtgcagtgatgaggtcacaacaaagcttagtatctcttatggtagaaactctggtgactatgaacacatccaggtcagatttaactacattagagaagaacattcagattgtaggaaattacataagagatgcaggattagcatctttcatgaacaccatcaagtatggtgtagaaacgaagatggccgccctgacactatcaaatctgagaccagatataaacaaattgagaagccttgttgatatctatctatcaaagggagcccgagccccttttatatgtatactcagagacccagttcatggagactttgcccctggaaactatccagcactgtggagctacgcaatgggcgttgctgtggtacaaaacaaagctatgcaacagtatgtaactggaagaacatatttggacatggaaatgttcctacttggacaagctgtagctaaagatgctgattccaaaatcagcagtgctctggaggaagaactaggtgtgacagatacagcaaaagagagactaagacaccatctgacaaacctttcaggaggggatggtgcgtaccacaagcctacaggtggtggagctatagaagtggcaattgatcatacagacataacatttggagtcgaggacactgctgatcgggacaacaagaactggacaaatgacagcaatgaaagatggatgaatcactcgatcagcaaccacacaatcacgattcgtggtgcagaagaacttgaagaagagacaaatgatgaagacatcactgatatagaaaacaagattgcacgaaggctggccgacagaaaacagagactaagccaggcaaacaataaacgagacaccagcagtgatgctgactatgagaatgatgatgatgctacagcggctgcagggataggaggaatttaacaggatacttggacaatagaagccagatcaaaagtaagaaaaacttagggtgaatgacaattcacagatcagctcaaccagacaccaccagcatacacgaaaccaaccttcacagtggatacctcagcatccaaaactctccttcccgaatggatcaggatgccttcttttttgagagggatcctgaagccgaaggagaggcaccacgaaaacaagaatcactctcagatgtcatcggactccttgacgtcgtcttatcctacaagcccacagaaattggagaagacagaagctggctccatggtatcatcgacaacccaaaagaaaacaagccatcatgcaaagccgacgataacaacaaagacagagcaatctcaacgtcgacccaagatcatagatcaagtgaggggagtggaatctctaggagaacaagtgagtcaaaaacagagacacatgctagaatccttgatcaacaaggtatacacagggcctctaggcgaggaactagtccaaaccctctacctgagaatatgggcaatgaaagaaacaccagaatcgacgaagattctccaaatgagagaagacatcagagatcagtacttacggatgaagacagaaagatggctgagaactctaataagagggaagaagaccaagttgagggatttccagaagaggtacgaagaagtacacccttatctgatgatggagagggtagaacaaataataatggaagaagcatggaaactagcagcacacatagtacaagaataactgatgtcattaccaacccaagtccagagcttgaagatgccgttctacaaaggaacaaaagacggccgacgaccatcaagcgtaaccaaacaagatcagagagaacacagagttcagaactccacaaatcaacaagtgaaaatagctccaacctcgaagaccacaacaccaaaaccagcccaaaagttccaccgtcaaagaacgaagagtcagcagccactccaaagaacaaccacaaccacagaaaaacaagatacacaacaaacaatgcaaacaacaacacaaaaagtccaccaactcccgaacacgacgcaaccgcaaatgaagaggaaaccagcaacacatcggtcgatgagatggccaagttattagtaagtcttggtgtaatgaaatcacaacatgaatttgaattatctaggagtgcaagtcatgtatttgctaagcgcatgttaaaatctgcaaattacaaagaaatgacatttaatctctgtggtatgcttatatcagttgaaaaatcacttgagaataaagtagaagaaaatagaacattacttaaacaaattcaagaggaaataaattcatccagggatcttcacaaacggttctcggaataccaaaaagaacagaactcactcatgatggccaatctatccacactccatataattacagatagaggcgggaaaacgggaaatcccagtgatactacaaggtcaccatcagtcttcacaaaagggaaagacaataaggtcaaaaagacaaggtttgacccctctatggaagctctaggaggtcaagagttcaagcctgacttgataagagaggatgaactgagagatgacatcaaaaatccggtactagaagaaaacaacaatgagcctcaagcatccaatgcatcacgcctgattccgtccactgaaaaacacactctgcactcactcaaactagttatcgaaaacagtcctctaagcagagtagagaagaaggcttacatcaaatccctttataagtgtcggacaaaccaagaggttaaaaatgtaatggagctattcgaggaagacatagattcactaactaactaaacatgaatctacaatttcaaccagcaatcaaaatcaatatccagagccaactcaaaaagctccctcaaaacaattaagaaaaacttagggtcaaagaaattttgcccggagaaaggaaatggctgaaacatacaggttccccagattctcacacgaagaaaatgggacagtagaacctctccctctcaaaacaggtcctgacaaaaaagcaatccctcacatcagaatagtcaaggtaggagatcctccaaaacatggagtcaggtatcttgatgtgctactattgggattctttgaaacacctaagcaaggacctctatctggcagcatatctgatctcacagaatcaaccagttattcaatctgtggatccggatccttaccaattggcatagccaagtattacggcacagatcaagaattattaaaagcctgcattgacctcaaaataactgtacgaagaacagttagatctggagaaatgatagtatacatggtagattcgatccatgctcctctactaccatggtccagccgactgagacaagggatgatatataatgccaataaagtagctctagcacctcaatgtctcccagtcgacaaagacatcagattcagggttgtatttgtcaatggaacatcactaggtacaattacaattgctaaggtcccaaaaactcttgcagatcttgcattaccgaactcaatatcagtgaatctgctggttacacttagggcaggagtatcaacggaacaaaaaggaatcctccccgttctagacgatgatggagaaaagaagctcaacttcatggtacacctaggaatcataagaagaaaagttgggaagatatattcagttgaatactgcaaaaataaaattgagaagatgaagctaatattctctctcgggcttgtaggtggaataagtttccatgtacatgcaacaggcacattatccaaaactctaatgagccaacttgcatggaaaaaagcagtttgctatcctttaatggatgtaaatccacatatgaatctagtcatctgggcagcttcagtagaaatcacaagtgtcgatgctgtgttccaacctgcaattccgaaagaatttcgctattacccaaatgttgttgcaaaaagcatcgggaaaatcaggaggatataagtctacactcctcaataatgacacccattagctctaaatcgtaccattaatcaaatacagatcaattcgatacaatcagttcaaataagaaaaacgtagggacaaagtcctctaccaacatcaaggaagacaagagtctcaaaaagctcagcctaagcagagagaaaaacaacaacacaaagaaagaaaaggacaagatcacaaacaagaacaaaagcaaaaacaaaaacaagaacaaaaaagggaagaaaaacaaaagtatacacaaaaaccaaaaaagaaaaaaggccagagacaaaaacggaggcaagaacaaaaatttaaacaaaaacagaatttaaattcataataaacaccaagatagagacaaaaatgcaaaaatcagagatcctcttcttagtatactcaagcttgctattatcttcatcattatgtcaaattccggtagaaaaactttcaaatgtaggggttataatcaatgagggcaaattacttaaaatagcaggatcttatgaatctagatacatagtgttaagcttggtaccttcaattgacctacaagatggatgtggaacaactcaaattattcaatacaagaatttattaaatagacttctaattcctctgaaggatgccttggatcttcaggaatccctgataacaataactaatgacaccactgtgacaaatgataatccacaaactagattctttggtgctgtcattggtaccatagcactaggagtagccacagctgctcaaataactgcgggcattgcattagctgaagcacgagaagccaggaaggacatagcactaataaaagattccatagtcaagacacacaattctgtagaactcattcaaagaggtataggagaacagataattgcattaaagacattacaagattttgtaaatgacgagataagacctgcaataggagaactaaggtgtgagactacggcattgaaactagggatcaagctcacccaacactactctgaattagcaacagcattcagctccaatcttgggactataggagaaaaaagtcttaccttgcaggcattatcatctctctactctgctaatataacagaaattctaagtacaactaaaaaggataaatcagatatatatgacatcatttacactgaacaggttaagggaactgtgatagatgttgatttggaaaaatacatggttaccctcttagttaaaataccaattttatcagaaataccaggcgtgttgatatacagagcttcatctatatcttataatattgaaggagaagaatggcatgtcgcaatcccaaattacataatcaataaggcatcatccttaggaggtgcagatgtcacaaactgtatagaatcaaaattggcatatatatgtcctagagatcctacacaattaatacctgataaccaacagaagtgtatactcggggatgtatcaaagtgccctgtgactaaagtaataaacaatctagtaccaaagttcgcattcatcaatggtggtgtagtggctaattgcattgcatccacatgtacatgcgggacaaacagaataccagtgaatcaagatcgctcaagaggagttacattcttgacctataccaattgcggtttaataggtataaatggaatagaactatatgccaataaaaggggacgagacactacttgggggaatcaaatcatcaaagtgggtccagcagtctccattagacctgtagacatttctttaaatcttgcatctgccacaaatttcctagaggaatccaagacagagctcatgaaggcaagggcaatcatatcagcagttggaggatggcacaacacagagagtactcagataatcatgataataattgtgtgcatacttataataatcatatgtggtatattatactatctatacagggttagaagactattagtaatgattaattcaactcataattcacctgttaatgcttatactctggagtcaagaatgagaaatccctacatgggtaacaactccaattaaaaaatcagatcaagtacattgtagcatacatacaacaatcaaatctatccacaacttcaccaatcaggtgtacaacaagtaagaaaaacttagggttaaagacaatccagtcaacctataaggcaacagcatccgattatacaaacgatggctgaaaaagggaaaacaaatagttcatattggtctacaacccgaaatgacaattccacggtaaacacacacattaatacaccagcaggaaggacacacatctggctactgattgcaacaacaatgcatacagtattgtccttcattatcatgatcctatgcattgacctaattataaaacaagacacttgtatgaagacaaacatcatgacagtatcctccatgaacgaaagtgccaaaataatcaaagagacaatcacagaattaatcagacaagaagtaatatcaaggaccataaacatacaaagttcagtacaaagcgggatcccaatattgttaaacaagcaaagcagagatctcacacaattaatagagaagtcatgcaacagacaggaattggctcagatatgcgaaaacaccattgctattcaccatgcagacggcatatctcctctggacccacacgatttctggagatgtcccgtaggggaacccctactgagcaacaaccccaatatctcattattacctggaccaagtctactttctggatccaccacaatttcaggatgtgttagactaccttcattatcaattggtgatgcaatatatgcgtattcatcaaacttaatcactcaaggatgtgcagatatagggaagtcatatcaggttttacaattaggttacatatccttaaattcagatatgtatcctgatttaaacccggtaatttctcatacctatgacatcaacgacaacaggaaatcatgttctgtaatagctgcaggaacaaggggttatcagttatgctccttgcccactgtgaatgagactacagactactcgagtgaaggtatagaagatttagtatttgacatattagatctcaagggaaagaccaaatctcatcgatacaaaaatgaagatataacttttgaccatcctttttctgcaatgtatccgagtgtaggaagtgggataaaaattgaaaatacactcattttcctagggtacggtggcttaacaactccgctccaaggcgacactaagtgtgtgataaacagatgtaccaatgttaatcagagtgtttgcaatgatgctcttaagataacttggctaaagaaaagacaagttgtcaatgtcttaattcgtatcaataattatttatctgataggccaaagattgttgtcgagacaattccaataactcaaaattacttaggtgccgaaggtaggctacttaaactaggtaaaaagatctacatatatactagatcttcaggttggcactccaacctgcaaataggatcattagatatcaacaaccccatgaccattaaatgggcgcctcatgaagtcctgtctcgaccaggaaaccaagactgcaactggtacaacagatgtccgagagaatgcatatcaggtgtatatactgatgcatatccactatctcctgatgcagtcaatgttgctacaaccacactgtacgcaaacacatcacgtgttaatcccaccataatgtactcaaatacctcagaaattatcaacatgctaagactcaagaatgtacaactagaggcagcatacactactacatcatgtatcactcatttcgggaagggctactgcttccacattgttgaaatcaaccaagccagccttaataccttacaacctatgttgttcaagacaagtatccctaaaatatgtaaaatcacatcttgagcagatcaagacccaacactatatcaattatgtgaaaaccagatatgatgtataaaaatttaaaaacaaagcatgaatagacatttatatgacaaatagaataagaaaaacttagggttaatgcctgcctatttgtcaaatatggataaacaggagtcaactcagaattcctcagacatcttatatccagaatgtcacttgaactctccgattgtaaaaagcaagattgctcaacttcacgttttgctagatatcaatcaaccctatgatttaaaagataacagtataataaatatcaccaaatacaaaatcagaaatggaggtttatcgccccggcagatcaaaatcagatcgctaggcaaaatccttaaacaagaaattaaggatattgatcgttacacttttgaaccttatccgattttctcattagagttactcagactggatatcccagaaatatgtgacaaaataagatccattttttcagtctctgatagattaataagagaactatcatctggatttcaagaattgtggttaaatattcttagacaattaggctgtgttgaagggaaagagggatttgactcattaaaggatgtagatatcatcccagatataactgataaatataataaaaacacatggtatcgcccattcttaacatggtttagcatcaaatatgatatgagatggatgcaaaagaataagtcggggaaccatttagatgtctcaaattctcacaattttcttgactgtaaatcatatattttgattatatatagagatttagtgataataataaataaattaaaattaaccggttatgtccttacacctgaattagtattaatgtattgtgatgttgtcgaaggaagatggaatatgtcttcagctggacgactcgataaaaggtcatcaaaaataacatgtaagggggaagaattatgggagcttatcgactctttatttcccaatcttggtgaggatgtatataatattatatcactactagaacctttatcacttgctttaatacagttggatgaccctgtaactaatttaaaaggagctttcatgagacatgttttgactgagctacatacaattttaataaaagataatatatacacagattcagaagcagacagcataatggaatcattgataaagattttcagagagacatcaattgatgaaaaagcagaaattttctccttttttagaacgtttggacatcctagcttagaagcaataactgctgccgataaagtaaggacacatatgtattcctccaaaaaaatcatactaaagacactatatgagtgtcatgcaatcttctgtgcaattataataaacggatatagagaaagacacggtggtcaatggccgccatgcgaattccccaatcatgtatgtcttgaactcaagaatgcacaaggatccaactctgcaatttcgtatgaatgtgccgtagacaattatagtagttttataggatttaaatttttaaaatttattgagcctcaattagatgaagatttgacaatttatatgaaggataaggctctatcacctaggaaagcagcatgggattcagtatatcccgacagtaatttatattacaaagtccctgaatcagaagagactcgtaggttaatcgaggtttttataaatgataataattttaaccctgcggatattattaattatgtagagtcaggagaatggttaaatgacgatagcttcaacatatcttacagtctcaaagaaaaagaaattaaacaagagggtcgactctttgccaagatgacatataagatgagagcagtccaggtattagcagaaacactactagcaaaaggagtaggtgagttattcagtgaaaatgggatggtaaagggagaaattgacctactaaagagactgactacattatctgtctcaggtgttccaagatccaactcagtttacaataatcccatattacatgagaaattgatcaaaaatatgaataagtgcaattcaaatgggtattgggatgaaagaaagaaatctaaaaatgaattcaaagctgcagactcatcaaccgaggggtatgagactctgagctgttttttaaccaccgatttgaaaaaatactgtctcaactggagatttgaaagtacagcgttgttcggtcaaagatgtaatgagatattcgggtttaaaactttctttaactggatgcaccctattctagaaaaaagtacaatttatgtaggagatccttactgtccagtacctgatagaatgcacaaagaactccaagatcatgatgataccggaatctttatccataatccaagagggggaatagagggttattgccagaaattatggacactaatctctattagtgcaatccatcttgcagctgttaaagttggtgtcagagtgtcagcaatggtacaaggagacaatcaagctatagcagtgacatccagagttcctgtcacacaaacctataagcaaaaaaagactcacgtctatgaagaaatcacaagatatttcggtgccttgagagaagttatgtttgatattggacatgaattaaaattaaatgagaccattataagtagcaaaatgtttgtatacagcaaacggatatattatgatgggaaaatcctcccacagtgcctcaaagctttaacaagatgtgtattttggtcagagactcttgtagatgaaaacaggtcagcatgctcaaacattgcaacatctatagccaaagctattgagaatggatattcacctatcttaggctattgtattgctctttttaaaacttgccaacaggtatgtatatcattaggaatgaccattaatcctactattacgtcaactatcaaagatcaatattttaaagggaaaaattggttaagatgtgcaatattgatcccagctaacataggagggttcaactatatgtctacagctagatgttttgtcagaaatataggtgatccagcagttgcagctctagcagacttaaagagattcatcaaagcaggtctgttagataaacaggtattatatcgtgtgatgaatcaagaaccaggagactcaagcttcttagattgggcatcagacccttattcatgcaatctcccacactcacaaagtataacaactataatcaaaaatgtaacagctagatcagtattgcaggaatcacctaatcctctcctatcaggtctcttttcagaatcaagtagtgaagaagatctcaacttagcatcatttttgatggataggaaagccatattgcccagagtagctcacgagatcttagataactcacttacaggtgtaagagaagctatagccgggatgcttgatacaacgaaatctctagtaagagctagtgtcaggagaggaggattatcatatagtatcttaagaagacttataaattatgatctattacaatatgagaccttaacaaggacactcagaaaaccggttaaggataatatagaatatgagtatatgtgttcagtagaattggcaataggattgaggcaaaaaatgtggtttcatctaacttatggaagaccaatccacggtttagaaactccagacccgttagaattattaagaggatcatttattgaaggctcagaaatatgtaaattttgtagatcagaagggaataaccctatgtatacttggttctatcttcctgacaacatcgacttagatacacttagcaatggaagtcctgccatacgtatcccttattttggttctgctactgatgaaagatcagaggctcaactaggttatgttaagaacttaagcaagccggcaaaagcagcaataagaatcgcaatggtttacacttgggcttatggaactgatgaaatatcatggatggaagcagcacttatagctcaaaccagggctaacttaagtttagagaatttgaagttactcacccctgtatcgacttctacaaatttgtcccacagattgagagatactgctacacagatgaaattttcaagtgctactttagttcgagcgagtcgatttattaccatatctaatgataatatggcattaaaagaggcaggagagtctaaagatactaatttagtttatcaacaaattatgttaaccggattgagcttatttgaattcaatatgaggtataaacaaggatcattatctaaacctatgatattacacttacatttgaataataaatgctgtatcatagaatctcctcaagaattgaatattcctcctagatctacattggacttagagatcactcaggaaaataacaagttaatctatgatcctgatcctctcaaggacatagatctagagttatttagtaaggttagggatgtagtacacacaattgatatgaattattggtctgatgatgaaataattagagcaactagtatatgtacagctatgactattgcagacacaatgtctcaattagatagagacaatcttaaagaaatgatagcactgataaatgatgatgatataaatagtttaatcaccgaatttatggttattgatatacccttattttgttccactttcgggggtattctaatcaatcaatttgcatattcactttacgggttaaacgtcagagggagggatgaaatatggggatatgtgatacgcataattaaagacacatcacatgcagtcctaaaagtactgtccaatgcattatcacatcctaaaatattcaaacgattctgggatgcaggagttgtagagcctgtttatggacctaacttgtccaatcaagacaagatactgttagccatttcagtatgtgaatactctgttgacctcttcatgcgtgattggcaagagggcataccgcttgaaatatttatttgtgataacgacccaaatatagcagaaatgagaaaactttcatttttagctagacatctagcatacttgtgtagtttggcagagatagctaaagagggaccaaaattggaatctatgacatctctcgaacgactcgaatcattgaaagagtatctagaacttacttttttagacgatcctatattaagatatagtcaattgacaggcttagttattaagatattcccttcaacgttaacttacatcaggaaatcttcaattaaggtgttgagagtaagaggtatagggataccagaagtcttagaggactgggatcctgatgccgatagtatgctactagataatataactgctgaggttcaacacaatatacctttaaagaagaacgaaagaactcccttctgggggttaagggtatcaaaatcacaagttctgcgacttagaggttatgaagagataaaaagggaagaaagaggaagatcaggtgtaggattaactctaccttttgatgggcgatatttatcacaccaattgagacttttcgggattaatagcaccagttgtttgaaagcattggaacttacctatttactgaatcctctagtcaataaggataaagatagattatatctcggagaaggtgcaggtgcaatgctgtcttgttatgatgctacattaggaccctgcatgaactattataattcaggtgttaattcttgtgatctcaacggacaaagagaattaaatatttatccttcagaagtggcactggtagggaagaaattgaataatgtcacgagtttatgtcaaagagttaaggttttattcaatgggaatcctggatcaacttggatagggaatgatgaatgtgaaacactaatctggaatgaattacagaataattcaatagggtttattcattgtgacatggaaggtggagaacacaaatgtgatcaggtggtcttacatgaacattatagtgtgatcaggattgcataccttgttggggataaggacgttatcttagtaagcaaaattgcaccaagattaggtacagactggacaaaacaattaagtttgtatttaagatactggagagatgtcagcttaatagtgttgaaaacatctaacccagcctctacagaaatgtatctgatatcaaaagatcctaaatctgatattatagaggatagtaatacagtattggcaaaccttcttccattatctaaagaggatagtattaagatagaaaaatggattctagttgagaaagccaaagttcatgattggatagttagagaattaaaggaagggagtgcatcgtcaggtatgctaagaccttaccatcaagcattacaaatcttcggatttgagcctaatttaaacaaattatgtagagatttcttatctacactaaatatagtagacacaaaaaattgtattatcacatttgatagagtattaagagatacaatctttgagtggactcggataaaagacgcagataagaagctaagacttacaggtaaatatgatctatatcctcttagagattcaggtaagttaaaagttatttctagaaggcttgtaatatcttggatagcattgtctatgtctacaagactagtaacagggtcatttccagacattaaatttgaatcaagactccaattaggtatagtatcaatatcctctcgtgaaatcaaaaatcttagggttatatcaaagattgtcattgacaaatttgaagatattatacatagtgtgacctataggttcttgactaaagaaataaaaatattgatgaaaattttgggagcagtcaaattatttggggcaagacagagcacatctgctgatatcactaatatcgatacatcggactccatacaatgatcttatatcttctcatctttattatctaatttgtttaaagagatgagttaacaagataagaaatccctttaactgactcataaaaacatagtaagaaaaacttacaacagacaagagtattaataatatatcgatatttcttaaactcttgtctggt

SEQ ID NO: 2 - DNA, 16242 bp, Human parainfluenza virus
accaaacaagaggaaaaacttgtttggaatatataataatattaaatagtattttagggttaaagtaatactttaaagggacaagtcacagacatttgatcttagtataaatacgcgtaatggccgggctactaagtacttttgacacatttagttccaggagaagtgagagcatcaataagtctggcggaggagcaattatacctggtcaaagaagtaccgtttctgtcttcacattaggcccgagtgtgacagatgatgcagataaattattaatagcaaccactttcttagcccactcattggacacagataaacaacactctcaaagaggaggatttttagtatcactccttgcaatggcctacagtagtccggagttatatctcactacaaacggtgtcaatgctgatgtcaagtatgtgatatacaatatagagagagatcctaaaagaacaaaaacagatgggttcattgtcaaaacaagagacatggagtatgaaagaacaacagagtggttgtttggacctatgatcaacaagaacccattgttccaagggcaaagagagaatgcggatctagaggcattgcttcagacatatggatatcctgcatgtctcggagctataatagttcaagtttggatagtgttggttaaagccataacaagtagtgctggtctaagaaaaggattcttcaatagattagaagcattcagacaggatggaaccgttaaaagtgccctggtcttcacaggagacacagttgaaggtattggtgcagtgatgaggtcacaacaaagcttagtatctcttatggtagaaactctggtgactatgaacacatccaggtcagatttaactacattagagaagaacattcagattgtaggaaattacataagagatgcaggattagcatctttcatgaacaccatcaagtatggtgtagaaacgaagatggccgccctgacactatcaaatctgagaccagatataaacaaattgagaagccttgttgatatctatctatcaaagggagcccgagccccttttatatgtatactcagagacccagttcatggagactttgcccctggaaactatccagcactgtggagctacgcaatgggcgttgctgtggtacaaaacaaagctatgcaacagtatgtaactggaagaacatatttggacatggaaatgttcctacttggacaagctgtagctaaagatgctgattccaaaatcagcagtgctctggaggaagaactaggtgtgacagatacagcaaaagagagactaagacaccatctgacaaacctttcaggaggggatggtgcgtaccacaagcctacaggtggtggagctatagaagtggcaattgatcatacagacataacatttggagtcgaggacactgctgatcgggacaacaagaactggacaaatgacagcaatgaaagatggatgaatcactcgatcagcaaccacacaatcacgattcgtggtgcagaagaacttgaagaagagacaaatgatgaagacatcactgatatagaaaacaagattgcacgaaggctggccgacagaaaacagagactaagccaggcaaacaataaacgagacaccagcagtgatgctgactatgagaatgatgatgatgctacagcggctgcagggataggaggaatttaacaggatacttggacaatagaagccagatcaaaagtaagaaaaacttagggtgaatgacaattcacagatcagctcaaccagggcgcgccagcatacacgaaaccaaccttcacagtggatacctcagcatccaaaactctccttcccgagtggattaggatgcctagttttttgagagggatcctgaagccgaaggagaggcaccacgaaaacaagaatcactctcagatgtcatcggactccttgacgtcgtcttatcctacaagcccacagaaattggagaagacagaagctggctccatggtatcatcgacaacccaaaagaaaacaagccatcatgcaaagccgacgataacaacaaagacagagcaatctcaacgtcgacccaagatcatagatcaagtgaggagagtggaatctctaggagaacaagtgagtcaaaaacagagacacatgctagaatccttgatcaacaaggtatacacagggcctctaggcgaggaactagtccaaaccctctacctgagaatatgggcaatgaaagaaacaccagaatcgacgaagattctccaaatgagagaagacatcagagatcagtacttacggatgaagacagaaagatggctgagaactctaataagagggaagaagaccaagttgagggatttccagaagaggtacgaagaagtacacccttatctgatgatggagagggtagaacaaataataatggaagaagcatggaaactagcagcacacatagtacaagaataactgatgtcattaccaacccaagtttaattaagaaaaacttagggttaaagcccaacatggatcaggacgccttcttttttgagagggatcctgaagccgaaggagaggcaccacgaaaacaagaatcactctcagatgtgatcggactccttgacgtagtcttatcctacaagcccacagaaataggagaagacagaagctggctccatggtatcatcgacaacccaaaagaaaacaagccatcatgcaaagccgacgataacaacaaagacagagcaatctcaacgtcgacccaagatcatagatcaagtgaggagagtggaatctctaggagaacaagtgagtcaaaaacagagacacatgctagaatccttgatcaacaaggtatacacagggcctctaggcgaggaactagtccaaaccctctacctgagaatatgggcaatgaaagaaacaccagaatcgacgaagattctccaaatgagagaagacatcagagatcagtacttacggatgaagacagaaagatggctgagaactctaataagagggaagaagaccaagttgagggatttccagaagaggtacgaagaagtacacccttatctgatgatggagagggtagaacaaataataatggaagaagcatggaaactagcagcacacatagtacaagaataactgatgtcattaccaacccaagtccagagcttgaagatgccgttctacaaaggaacaaaagacggccgacgaccatcaagcgtaaccaaacaagatcagagagaacacagagttcagaactccacaaatcaacaagtgaaaatagctccaacctcgaagaccacaacaccaaaaccagcccaaaagttccaccgtcaaagaacgaagagtcagcagccactccaaagaacaaccacaaccacagaaaaacaagatacacaacaaacaatgcaaacaacaacacaaaaagtccaccaactcccgaacacgacgcaaccgcaaatgaagaggaaaccagcaacacatcggtcgatgagatggccaagttattagtaagtcttggtgtaatgaaatcacaacatgaatttgaattatctaggagtgcaagtcatgtatttgctaagcgcatgttaaaatctgcaaattacaaagaaatgacatttaatctctgtggtatgcttatatcagttgaaaaatcacttgagaataaagtagaagaaaatagaacattacttaaacaaattcaagaggaaataaattcatccagggatcttcacaaacggttctcggaataccaaaaagaacagaactcactcatgatggccaatctatccacactccatataattacagatagaggcgggaaaacgggaaatcccagtgatactacaaggtcaccatcagtcttcacaaaagggaaagacaataaggtcaaaaagacaaggtttgacccctctatggaagctctaggaggtcaagagttcaagcctgacttgataagagaggatgaactgagagatgacatcaaaaatccggtactagaagaaaacaacaatgagcctcaagcatccaatgcatcacgcctgattccgtccactgaaaaacacactctgcactcactcaaactagttatcgaaaacagtcctctaagcagagtagagaagaaggcttacatcaaatccctttataagtgtcggacaaaccaagaggttaaaaatgtaatggagctattcgaggaagacatagattcactaactaactaaacatgaatctacagctagcgcggccgcaaaacaattaagaaaaacttagggtcaaagaaattttgcccggagaaaggaaatggctgaaacatacaggttccccagattctcacacgaagaaaatgggacagtagaacctctccctctcaaaacaggtcctgacaaaaaagcaatccctcacatcagaatagtcaaggtaggagatcctccaaaacatggagtcaggtatcttgatgtgctactattgggattctttgaaacacctaagcaaggacctctatctggcagcatatctgatctcacagaatcaaccagttattcaatctgtggatccggatccttaccaattggcatagccaagtattacggcacagatcaagaattattaaaagcctgcattgacctcaaaataactgtacgaagaacagttagatctggagaaatgatagtatacatggtagattcgatccatgctcctctactaccatggtccagccgactgagacaagggatgatatataatgccaataaagtagctctagcacctcaatgtctcccagtcgacaaagacatcagattcagggttgtatttgtcaatggaacatcactaggtacaattacaattgctaaggtcccaaaaactcttgcagatcttgcattaccgaactcaatatcagtgaatctgctggttacacttagggcaggagtatcaacggaacaaaaaggaatcctccccgttctagacgatgatggagaaaagaagctcaacttcatggtacacctaggaatcataagaagaaaagttgggaagatatattcagttgaatactgcaaaaataaaattgagaagatgaagctaatattctctctcgggcttgtaggtggaataagtttccatgtacatgcaacaggcacattatccaaaactctaatgagccaacttgcatggaaaaaagcagtttgctatcctttaatggatgtaaatccacatatgaatctagtcatctgggcagcttcagtagaaatcacaagtgtagatgctgtgttccaacctgcaattccgaaagaatttcgctattacccaaatgttgttgcaaaaagcatcgggaaaatcaggaggatataagtctacactcctcaataatgacacccattagctctaaatcgtaccattaatcaaatacagatcaattcgatacaatcagttcaaataagaaaaacgtagggacaaagtcctctaccaacatcaaggaagacaagagtctcaaaaagctcagcctaagcagagagaaaaacaacaacacaaagaaagaaaaggacaagatcacaaacaagaacaaaagcaaaaacaaaaacaagaacaaaaaagggaagaaaaacaaaagtatacacaaaaaccaaaaaagaaaaaaggccagagacaaaaacggaggcaagaacaaaaatttaaacaaaaacagaatttaaattcataataaacaccaagatagagacaaaaatgcaaaaatcagagatcctcttcttagtatactcaagcttgctattatcttcatcattatgtcaaattccggtagaaaaactttcaaatgtaggggttataatcaatgagggcaaattacttaaaatagcaggatcttatgaatctagatacatagtgttaagcttggtaccttcaattgacctacaagatggatgtggaacaactcaaattattcaatacaagaatttattaaatagacttctaattcctctgaaggatgccttggatcttcaggaatccctgataacaataactaatgacaccactgtgacaaatgataatccacaaactagattctttggtgctgtcattggtaccatagcactaggagtagccacagctgctcaaataactgcgggcattgcattagctgaagcacgagaagccaggaaggacatagcactaataaaagattccatagtcaagacacacaattctgtagaactcattcaaagaggtataggagaacagataattgcattaaagacattacaagattttgtaaatgacgagataagacctgcaataggagaactaaggtgtgagactacggcattgaaactagggatcaagctcacccaacactactctgaattagcaacagcattcagctccaatcttgggactataggagaaaaaagtcttaccttgcaggcattatcatctctctactctgctaatataacagaaattctaagtacaactaaaaaggataaatcagatatatatgacatcatttacactgaacaggttaagggaactgtgatagatgttgatttggaaaaatacatggttaccctcttagttaaaataccaattttatcagaaataccaggcgtgttgatatacagagcttcatctatatcttataatattgaaggagaagaatggcatgtcgcaatcccaaattacataatcaataaggcatcatccttaggaggtgcagatgtcacaaactgtatagaatcaaaattggcatatatatgtcctagagatcctacacaattaatacctgataaccaacagaagtgtatactcggggatgtatcaaagtgccctgtgactaaagtaataaacaatctagtaccaaagttcgcattcatcaatggtggtgtagtggctaattgcattgcatccacatgtacatgcgggacaaacagaataccagtgaatcaagatcgctcaagaggagttacattcttgacctataccaattgcggtttaataggtataaatggaatagaactatatgccaataaaaggggacgagacactacttgggggaatcaaatcatcaaagtgggtccagcagtctccattagacctgtagacatttctttaaatcttgcatctgccacaaatttcctagaggaatccaagacagagctcatgaaggcaagggcaatcatatcagcagttggaggatggcacaacacagagagtactcagataatcatgataataattgtgtgcatacttataataatcatatgtggtatattatactatctatacagggttagaagactattagtaatgattaattcaactcataattcacctgttaatgcttatactctggagtcaagaatgagaaatccctacatgggtaacaactccaattaaaaaatcagatcaagtacattgtagcatacatacaacaatcaaatctatccacaacttcaccaatcaggtgtacaacaagtaagaaaaacttagggttaaagacaatccagtcaacctataaggcaacagcatccgattatacaaacgatggctgaaaaagggaaaacaaatagttcatattggtctacaacccgaaatgacaattccacggtaaacacacacattaatacaccagcaggaaggacacacatctggctactgattgcaacaacaatgcatacagtattgtccttcattatcatgatcctatgcattgacctaattataaaacaagacacttgtatgaagacaaacatcatgacagtatcctccatgaacgaaagtgccaaaataatcaaagagacaatcacagaattaatcagacaagaagtaatatcaaggaccataaacatacaaagttcagtacaaagcgggatcccaatattgttaaacaagcaaagcagagatctcacacaattaatagagaagtcatgcaacagacaggaattggctcagatatgcgaaaacaccattgctattcaccatgcagacggcatatctcctctggacccacacgatttctggagatgtcccgtaggggaacccctactgagcaacaaccccaatatctcattattacctggaccaagtctactttctggatccaccacaatttcaggatgtgttagactaccttcattatcaattggtgatgcaatatatgcgtattcatcaaacttaatcactcaaggatgtgcagatatagggaagtcatatcaggttttacaattaggttacatatccttaaattcagatatgtatcctgatttaaacccggtaatttctcatacctatgacatcaacgacaacaggaaatcatgttctgtaatagctgcaggaacaaggggttatcagttatgctccttgcccactgtgaatgagactacagactactcgagtgaaggtatagaagatttagtatttgacatattagatctcaagggaaagaccaaatctcatcgatacaaaaatgaagatataacttttgaccatcctttttctgcaatgtatccgagtgtaggaagtgggataaaaattgaaaatacactcattttcctagggtacggtggcttaacaactccgctccaaggcgacactaagtgtgtgataaacagatgtaccaatgttaatcagagtgtttgcaatgatgctcttaagataacttggctaaagaaaagacaagttgtcaatgtcttaattcgtatcaataattatttatctgataggccaaagattgttgtcgagacaattccaataactcaaaattacttaggtgccgaaggtaggctacttaaactaggtaaaaagatctacatatatactagatcttcaggttggcactccaacctgcaaataggatcattagatatcaacaaccccatgaccattaaatgggcgcctcatgaagtcctgtctcgaccaggaaaccaagactgcaactggtacaacagatgtccgagagaatgcatatcaggtgtatatactgatgcatatccactatctcctgatgcagtcaatgttgctacaaccacactgtacgcaaacacatcacgtgttaatcccaccataatgtactcaaatacctcagaaattatcaacatgctaagactcaagaatgtacaactagaggcagcatacactactacatcatgtatcactcatttcgggaagggctactgcttccacattgttgaaatcaaccaagccagccttaataccttacaacctatgttgttcaagacaagtatccctaaaatatgtaaaatcacatcttgagcagatcaagacccaacactatatcaattatgtgaaaaccagatatgatgtataaaaatttaaaaacaaagcatgaatagacatttatatgacaaatagaataagaaaaacttagggttaatgcctgcctatttgtcaaatatggataaacaggagtcaactcagaattcctcagacatcttatatccagaatgtcacttgaactctccgattgtaaaaagcaagattgctcaacttcacgttttgctagatatcaatcaaccctatgatttaaaagataacagtataataaatatcaccaaatacaaaatcagaaatggaggtttatcgccccggcagatcaaaatcagatcgctaggcaaaatccttaaacaagaaattaaggatattgatcgttacacttttgaaccttatccgattttctcattagagttactcagactggatatcccagaaatatgtgacaaaataagatccattttttcagtctctgatagattaataagagaactatcatctggatttcaagaattgtggttaaatattcttagacaattaggctgtgttgaagggaaagagggatttgactcattaaaggatgtagatatcatcccagatataactgataaatataataaaaacacatggtatcgcccattcttaacatggtttagcatcaaatatgatatgagatggatgcaaaagaataagtcggggaaccatttagatgtctcaaattctcacaattttcttgactgtaaatcatatattttgattatatatagagatttagtgataataataaataaattaaaattaaccggttatgtccttacacctgaattagtattaatgtattgtgatgttgtcgaaggaagatggaatatgtcttcagctggacgactcgataaaaggtcatcaaaaataacatgtaagggggaagaattatgggagcttatcgactctttatttcccaatcttggtgaggatgtatataatattatatcactactagaacctttatcacttgctttaatacagttggatgaccctgtaactaatttaaaaggagctttcatgagacatgttttgactgagctacatacaattttaataaaagataatatatacacagattcagaagcagacagcataatggaatcattgataaagattttcagagagacatcaattgatgaaaaagcagaaattttctccttttttagaacgtttggacatcctagcttagaagcaataactgctgccgataaagtaaggacacatatgtattcctccaaaaaaatcatactaaagacactatatgagtgtcatgcaatcttctgtgcaattataataaacggatatagagaaagacacggtggtcaatggccgccatgcgaattccccaatcatgtatgtcttgaactcaagaatgcacaaggatccaactctgcaatttcgtatgaatgtgccgtagacaattatagtagttttataggatttaaatttttaaaatttattgagcctcaattagatgaagatttgacaatttatatgaaggataaggctctatcacctaggaaagcagcatgggattcagtatatcccgacagtaatttatattacaaagtccctgaatcagaagagactcgtaggttaatcgaggtttttataaatgataataattttaaccctgcggatattattaattatgtagagtcaggagaatggttaaatgacgatagcttcaacatatcttacagtctcaaagaaaaagaaattaaacaagagggtcgactctttgccaagatgacatataagatgagagcagtccaggtattagcagaaacactactagcaaaaggagtaggtgagttattcagtgaaaatgggatggtaaagggagaaattgacctactaaagagactgactacattatctgtctcaggtgttccaagatccaactcagtttacaataatcccatattacatgagaaattgatcaaaaatatgaataagtgcaattcaaatgggtattgggatgaaagaaagaaatctaaaaatgaattcaaagctgcagactcatcaaccgaggggtatgagactctgagctgttttttaaccaccgatttgaaaaaatactgtctcaactggagatttgaaagtacagcgttgttcggtcaaagatgtaatgagatattcgggtttaaaactttctttaactggatgcaccctattctagaaaaaagtacaatttatgtaggagatccttactgtccagtacctgatagaatgcacaaagaactccaagatcatgatgataccggaatctttatccataatccaagagggggaatagagggttattgccagaaattatggacactaatctctattagtgcaatccatcttgcagctgttaaagttggtgtcagagtgtcagcaatggtacaaggagacaatcaagctatagcagtgacatccagagttcctgtcacacaaacctataagcaaaaaaagactcacgtctatgaagaaatcacaagatatttcggtgccttgagagaagttatgtttgatattggacatgaattaaaattaaatgagaccattataagtagcaaaatgtttgtatacagcaaacggatatattatgatgggaaaatcctcccacagtgcctcaaagctttaacaagatgtgtattttggtcagagactcttgtagatgaaaacaggtcagcatgctcaaacattgcaacatctatagccaaagctattgagaatggatattcacctatcttaggctattgtattgctctttttaaaacttgccaacaggtatgtatatcattaggaatgaccattaatcctactattacgtcaactatcaaagatcaatattttaaagggaaaaattggttaagatgtgcaatattgatcccagctaacataggagggttcaactatatgtctacagctagatgttttgtcagaaatataggtgatccagcagttgcagctctagcagacttaaagagattcatcaaagcaggtctgttagataaacaggtattatatcgtgtgatgaatcaagaaccaggagactcaagcttcttagattgggcatcagacccttattcatgcaatctcccacactcacaaagtataacaactataatcaaaaatgtaacagctagatcagtattgcaggaatcacctaatcctctcctatcaggtctcttttcagaatcaagtagtgaagaagatctcaacttagcatcatttttgatggataggaaagccatattgcccagagtagctcacgagatcttagataactcacttacaggtgtaagagaagctatagccgggatgcttgatacaacgaaatctctagtaagagctagtgtcaggagaggaggattatcatatagtatcttaagaagacttataaattatgatctattacaatatgagaccttaacaaggacactcagaaaaccggttaaggataatatagaatatgagtatatgtgttcagtagaattggcaataggattgaggcaaaaaatgtggtttcatctaacttatggaagaccaatccacggtttagaaactccagacccgttagaattattaagaggatcatttattgaaggctcagaaatatgtaaattttgtagatcagaagggaataaccctatgtatacttggttctatcttcctgacaacatcgacttagatacacttagcaatggaagtcctgccatacgtatcccttattttggttctgctactgatgaaagatcagaggctcaactaggttatgttaagaacttaagcaagccggcaaaagcagcaataagaatcgcaatggtttacacttgggcttatggaactgatgaaatatcatggatggaagcagcacttatagctcaaaccagggctaacttaagtttagagaatttgaagttactcacccctgtatcgacttctacaaatttgtcccacagattgagagatactgctacacagatgaaattttcaagtgctactttagttcgagcgagtcgatttattaccatatctaatgataatatggcattaaaagaggcaggagagtctaaagatactaatttagtttatcaacaaattatgttaaccggattgagcttatttgaattcaatatgaggtataaacaaggatcattatctaaacctatgatattacacttacatttgaataataaatgctgtatcatagaatctcctcaagaattgaatattcctcctagatctacattggacttagagatcactcaggaaaataacaagttaatctatgatcctgatcctctcaaggacatagatctagagttatttagtaaggttagggatgtagtacacacaattgatatgaattattggtctgatgatgaaataattagagcaactagtatatgtacagctatgactattgcagacacaatgtctcaattagatagagacaatcttaaagaaatgatagcactgataaatgatgatgatataaatagtttaatcaccgaatttatggttattgatatacccttattttgttccactttcgggggtattctaatcaatcaatttgcatattcactttacgggttaaacgtcagagggagggatgaaatatggggatatgtgatacgcataattaaagacacatcacatgcagtcctaaaagtactgtccaatgcattatcacatcctaaaatattcaaacgattctgggatgcaggagttgtagagcctgtttatggacctaacttgtccaatcaagacaagatactgttagccatttcagtatgtgaatactctgttgacctcttcatgcgtgattggcaagagggcataccgcttgaaatatttatttgtgataacgacccaaatatagcagaaatgagaaaactttcatttttagctagacatctagcatacttgtgtagtttggcagagatagctaaagagggaccaaaattggaatctatgacatctctcgaacgactcgaatcattgaaagagtatctagaacttacttttttagacgatcctatattaagatatagtcaattgacaggcttagttattaagatattcccttcaacgttaacttacatcaggaaatcttcaattaaggtgttgagagtaagaggtatagggataccagaagtcttagaggactgggatcctgatgccgatagtatgctactagataatataactgctgaggttcaacacaatatacctttaaagaagaacgaaagaactcccttctgggggttaagggtatcaaaatcacaagttctgcgacttagaggttatgaagagataaaaagggaagaaagaggaagatcaggtgtaggattaactctaccttttgatgggcgatatttatcacaccaattgagacttttcgggattaatagcaccagttgtttgaaagcattggaacttacctatttactgaatcctctagtcaataaggataaagatagattatatctcggagaaggtgcaggtgcaatgctgtcttgttatgatgctacattaggaccctgcatgaactattataattcaggtgttaattcttgtgatctcaacggacaaagagaattaaatatttatccttcagaagtggcactggtagggaagaaattgaataatgtcacgagtttatgtcaaagagttaaggttttattcaatgggaatcctggatcaacttggatagggaatgatgaatgtgaaacactaatctggaatgaattacagaataattcaatagggtttattcattgtgacatggaaggtggagaacacaaatgtgatcaggtggtcttacatgaacattatagtgtgatcaggattgcataccttgttggggataaggacgttatcttagtaagcaaaattgcaccaagattaggtacagactggacaaaacaattaagtttgtatttaagatactggagagatgtcagcttaatagtgttgaaaacatctaacccagcctctacagaaatgtatctgatatcaaaagatcctaaatctgatattatagaggatagtaatacagtattggcaaaccttcttccattatctaaagaggatagtattaagatagaaaaatggattctagttgagaaagccaaagttcatgattggatagttagagaattaaaggaagggagtgcatcgtcaggtatgctaagaccttaccatcaagcattacaaatcttcggatttgagcctaatttaaacaaattatgtagagatttcttatctacactaaatatagtagacacaaaaaattgtattatcacatttgatagagtattaagagatacaatctttgagtggactcggataaaagacgcagataagaagctaagacttacaggtaaatatgatctatatcctcttagagattcaggtaagttaaaagttatttctagaaggcttgtaatatcttggatagcattgtctatgtctacaagactagtaacagggtcatttccagacattaaatttgaatcaagactccaattaggtatagtatcaatatcctctcgtgaaatcaaaaatcttagggttatatcaaagattgtcattgacaaatttgaagatattatacatagtgtgacctataggttcttgactaaagaaataaaaatattgatgaaaattttgggagcagtcaaattatttggggcaagacagagcacatctgctgatatcactaatatcgatacatcggactccatacaatgatcttatatcttctcatctttattatctaatttgtttaaagagatgagttaacaagataagaaatccctttaactgactcataaaaacatagtaagaaaaacttacaacagacaagagtattaataatatatcgatatttcttaaactcttgtctggt

SEQ ID NO: 3 - DNA, 16236 bp, Human parainfluenza virus
accaaacaagaggaaaaacttgtttggaatatataataatattaaatagtattttagggttaaagtaatactttaaagggacaagtcacagacatttgatcttagtataaatacgcgtaatggccgggctactaagtacttttgacacatttagttccaggagaagtgagagcatcaataagtctggcggaggagcaattatacctggtcaaagaagtaccgtttctgtcttcacattaggcccgagtgtgacagatgatgcagataaattattaatagcaaccactttcttagcccactcattggacacagataaacaacactctcaaagaggaggatttttagtatcactccttgcaatggcctacagtagtccggagttatatctcactacaaacggtgtcaatgctgatgtcaagtatgtgatatacaatatagagagagatcctaaaagaacaaaaacagatgggttcattgtcaaaacaagagacatggagtatgaaagaacaacagagtggttgtttggacctatgatcaacaagaacccattgttccaagggcaaagagagaatgcggatctagaggcattgcttcagacatatggatatcctgcatgtctcggagctataatagttcaagtttggatagtgttggttaaagccataacaagtagtgctggtctaagaaaaggattcttcaatagattagaagcattcagacaggatggaaccgttaaaagtgccctggtcttcacaggagacacagttgaaggtattggtgcagtgatgaggtcacaacaaagcttagtatctcttatggtagaaactctggtgactatgaacacatccaggtcagatttaactacattagagaagaacattcagattgtaggaaattacataagagatgcaggattagcatctttcatgaacaccatcaagtatggtgtagaaacgaagatggccgccctgacactatcaaatctgagaccagatataaacaaattgagaagccttgttgatatctatctatcaaagggagcccgagccccttttatatgtatactcagagacccagttcatggagactttgcccctggaaactatccagcactgtggagctacgcaatgggcgttgctgtggtacaaaacaaagctatgcaacagtatgtaactggaagaacatatttggacatggaaatgttcctacttggacaagctgtagctaaagatgctgattccaaaatcagcagtgctctggaggaagaactaggtgtgacagatacagcaaaagagagactaagacaccatctgacaaacctttcaggaggggatggtgcgtaccacaagcctacaggtggtggagctatagaagtggcaattgatcatacagacataacatttggagtcgaggacactgctgatcgggacaacaagaactggacaaatgacagcaatgaaagatggatgaatcactcgatcagcaaccacacaatcacgattcgtggtgcagaagaacttgaagaagagacaaatgatgaagacatcactgatatagaaaacaagattgcacgaaggctggccgacagaaaacagagactaagccaggcaaacaataaacgagacaccagcagtgatgctgactatgagaatgatgatgatgctacagcggctgcagggataggaggaatttaacaggatacttggacaatagaagccagatcaaaagtaagaaaaacttagggtgaatgacaattcacagatcagctcaaccagggcgcgccagcatacacgaaaccaaccttcacagtggatacctcagcatccaaaactctccttcccgagtggattaggatgcctagttttttgagagggatcctgaagccgaaggagaggcaccacgaaaacaagaatcactctcagatgtcatcggactccttgacgtcgtcttatcctacaagcccacagaaattggagaagacagaagctggctccatggtatcatcgacaacccaaaagaaaacaagccatcatgcaaagccgacgataacaacaaagacagagcaatctcaacgtcgacccaagatcatagatcaagtggtggaatctctaggagaacaagtgagtcaaaaacagagacacatgctagaatccttgatcaacaaggtatacacagggcctctaggcgaggaactagtccaaaccctctacctgagaatatgggcaatgaaagaaacaccagaatcgacgaagattctccaaatgagagaagacatcagagatcagtacttacggatgaagacagaaagatggctgagaactctaataagagggaagaagaccaagttgagggatttccagaagaggtacgaagaagtacacccttatctgatgatggagagggtagaacaaataataatggaagaagcatggaaactagcagcacacatagtacaagaataactgatgtcattaccaacccaagtttaattaagaaaaacttagggttaaagcccaacatggatcaggacgccttcttttttgagagggatcctgaagccgaaggagaggcaccacgaaaacaagaatcactctcagatgtgatcggactccttgacgtagtcttatcctacaagcccacagaaataggagaagacagaagctggctccatggtatcatcgacaacccaaaagaaaacaagccatcatgcaaagccgacgataacaacaaagacagagcaatctcaacgtcgacccaagatcatagatcaagtgaggagagtggaatctctaggagaacaagtgagtcaaaaacagagacacatgctagaatccttgatcaacaaggtatacacagggcctctaggcgaggaactagtccaaaccctctacctgagaatatgggcaatgaaagaaacaccagaatcgacgaagattctccaaatgagagaagacatcagagatcagtacttacggatgaagacagaaagatggctgagaactctaataagagggaagaagaccaagttgagggatttccagaagaggtacgaagaagtacacccttatctgatgatggagagggtagaacaaataataatggaagaagcatggaaactagcagcacacatagtacaagaataactgatgtcattaccaacccaagtccagagcttgaagatgccgttctacaaaggaacaaaagacggccgacgaccatcaagcgtaaccaaacaagatcagagagaacacagagttcagaactccacaaatcaacaagtgaaaatagctccaacctcgaagaccacaacaccaaaaccagcccaaaagttccaccgtcaaagaacgaagagtcagcagccactccaaagaacaaccacaaccacagaaaaacaagatacacaacaaacaatgcaaacaacaacacaaaaagtccaccaactcccgaacacgacgcaaccgcaaatgaagaggaaaccagcaacacatcggtcgatgagatggccaagttattagtaagtcttggtgtaatgaaatcacaacatgaatttgaattatctaggagtgcaagtcatgtatttgctaagcgcatgttaaaatctgcaaattacaaagaaatgacatttaatctctgtggtatgcttatatcagttgaaaaatcacttgagaataaagtagaagaaaatagaacattacttaaacaaattcaagaggaaataaattcatccagggatcttcacaaacggttctcggaataccaaaaagaacagaactcactcatgatggccaatctatccacactccatataattacagatagaggcgggaaaacgggaaatcccagtgatactacaaggtcaccatcagtcttcacaaaagggaaagacaataaggtcaaaaagacaaggtttgacccctctatggaagctctaggaggtcaagagttcaagcctgacttgataagagaggatgaactgagagatgacatcaaaaatccggtactagaagaaaacaacaatgagcctcaagcatccaatgcatcacgcctgattccgtccactgaaaaacacactctgcactcactcaaactagttatcgaaaacagtcctctaagcagagtagagaagaaggcttacatcaaatccctttataagtgtcggacaaaccaagaggttaaaaatgtaatggagctattcgaggaagacatagattcactaactaactaaacatgaatctacagctagcgcggccgcaaaacaattaagaaaaacttagggtcaaagaaattttgcccggagaaaggaaatggctgaaacatacaggttccccagattctcacacgaagaaaatgggacagtagaacctctccctctcaaaacaggtcctgacaaaaaagcaatccctcacatcagaatagtcaaggtaggagatcctccaaaacatggagtcaggtatcttgatgtgctactattgggattctttgaaacacctaagcaaggacctctatctggcagcatatctgatctcacagaatcaaccagttattcaatctgtggatccggatccttaccaattggcatagccaagtattacggcacagatcaagaattattaaaagcctgcattgacctcaaaataactgtacgaagaacagttagatctggagaaatgatagtatacatggtagattcgatccatgctcctctactaccatggtccagccgactgagacaagggatgatatataatgccaataaagtagctctagcacctcaatgtctcccagtcgacaaagacatcagattcagggttgtatttgtcaatggaacatcactaggtacaattacaattgctaaggtcccaaaaactcttgcagatcttgcattaccgaactcaatatcagtgaatctgctggttacacttagggcaggagtatcaacggaacaaaaaggaatcctccccgttctagacgatgatggagaaaagaagctcaacttcatggtacacctaggaatcataagaagaaaagttgggaagatatattcagttgaatactgcaaaaataaaattgagaagatgaagctaatattctctctcgggcttgtaggtggaataagtttccatgtacatgcaacaggcacattatccaaaactctaatgagccaacttgcatggaaaaaagcagtttgctatcctttaatggatgtaaatccacatatgaatctagtcatctgggcagcttcagtagaaatcacaagtgtcgatgctgtgttccaacctgcaattccgaaagaatttcgctattacccaaatgttgttgcaaaaagcatcgggaaaatcaggaggatataagtctacactcctcaataatgacacccattagctctaaatcgtaccattaatcaaatacagatcaattcgatacaatcagttcaaataagaaaaacgtagggacaaagtcctctaccaacatcaaggaagacaagagtctcaaaaagctcagcctaagcagagagaaaaacaacaacacaaagaaagaaaaggacaagatcacaaacaagaacaaaagcaaaaacaaaaacaagaacaaaaaagggaagaaaaacaaaagtatacacaaaaaccaaaaaagaaaaaaggccagagacaaaaacggaggcaagaacaaaaatttaaacaaaaacagaatttaaattcataataaacaccaagatagagacaaaaatgcaaaaatcagagatcctcttcttagtatactcaagcttgctattatcttcatcattatgtcaaattccggtagaaaaactttcaaatgtaggggttataatcaatgagggcaaattacttaaaatagcaggatcttatgaatctagatacatagtgttaagcttggtaccttcaattgacctacaagatggatgtggaacaactcaaattattcaatacaagaatttattaaatagacttctaattcctctgaaggatgccttggatcttcaggaatccctgataacaataactaatgacaccactgtgacaaatgataatccacaaactagattctttggtgctgtcattggtaccatagcactaggagtagccacagctgctcaaataactgcgggcattgcattagctgaagcacgagaagccaggaaggacatagcactaataaaagattccatagtcaagacacacaattctgtagaactcattcaaagaggtataggagaacagataattgcattaaagacattacaagattttgtaaatgacgagataagacctgcaataggagaactaaggtgtgagactacggcattgaaactagggatcaagctcacccaacactactctgaattagcaacagcattcagctccaatcttgggactataggagaaaaaagtcttaccttgcaggcattatcatctctctactctgctaatataacagaaattctaagtacaactaaaaaggataaatcagatatatatgacatcatttacactgaacaggttaagggaactgtgatagatgttgatttggaaaaatacatggttaccctcttagttaaaataccaattttatcagaaataccaggcgtgttgatatacagagcttcatctatatcttataatattgaaggagaagaatggcatgtcgcaatcccaaattacataatcaataaggcatcatccttaggaggtgcagatgtcacaaactgtatagaatcaaaattggcatatatatgtcctagagatcctacacaattaatacctgataaccaacagaagtgtatactcggggatgtatcaaagtgccctgtgactaaagtaataaacaatctagtaccaaagttcgcattcatcaatggtggtgtagtggctaattgcattgcatccacatgtacatgcgggacaaacagaataccagtgaatcaagatcgctcaagaggagttacattcttgacctataccaattgcggtttaataggtataaatggaatagaactatatgccaataaaaggggacgagacactacttgggggaatcaaatcatcaaagtgggtccagcagtctccattagacctgtagacatttctttaaatcttgcatctgccacaaatttcctagaggaatccaagacagagctcatgaaggcaagggcaatcatatcagcagttggaggatggcacaacacagagagtactcagataatcatgataataattgtgtgcatacttataataatcatatgtggtatattatactatctatacagggttagaagactattagtaatgattaattcaactcataattcacctgttaatgcttatactctggagtcaagaatgagaaatccctacatgggtaacaactccaattaaaaaatcagatcaagtacattgtagcatacatacaacaatcaaatctatccacaacttcaccaatcaggtgtacaacaagtaagaaaaacttagggttaaagacaatccagtcaacctataaggcaacagcatccgattatacaaacgatggctgaaaaagggaaaacaaatagttcatattggtctacaacccgaaatgacaattccacggtaaacacacacattaatacaccagcaggaaggacacacatctggctactgattgcaacaacaatgcatacagtattgtccttcattatcatgatcctatgcattgacctaattataaaacaagacacttgtatgaagacaaacatcatgacagtatcctccatgaacgaaagtgccaaaataatcaaagagacaatcacagaattaatcagacaagaagtaatatcaaggaccataaacatacaaagttcagtacaaagcgggatcccaatattgttaaacaagcaaagcagagatctcacacaattaatagagaagtcatgcaacagacaggaattggctcagatatgcgaaaacaccattgctattcaccatgcagacggcatatctcctctggacccacacgatttctggagatgtcccgtaggggaacccctactgagcaacaaccccaatatctcattattacctggaccaagtctactttctggatccaccacaatttcaggatgtgttagactaccttcattatcaattggtgatgcaatatatgcgtattcatcaaacttaatcactcaaggatgtgcagatatagggaagtcatatcaggttttacaattaggttacatatccttaaattcagatatgtatcctgatttaaacccggtaatttctcatacctatgacatcaacgacaacaggaaatcatgttctgtaatagctgcaggaacaaggggttatcagttatgctccttgcccactgtgaatgagactacagactactcgagtgaaggtatagaagatttagtatttgacatattagatctcaagggaaagaccaaatctcatcgatacaaaaatgaagatataacttttgaccatcctttttctgcaatgtatccgagtgtaggaagtgggataaaaattgaaaatacactcattttcctagggtacggtggcttaacaactccgctccaaggcgacactaagtgtgtgataaacagatgtaccaatgttaatcagagtgtttgcaatgatgctcttaagataacttggctaaagaaaagacaagttgtcaatgtcttaattcgtatcaataattatttatctgataggccaaagattgttgtcgagacaattccaataactcaaaattacttaggtgccgaaggtaggctacttaaactaggtaaaaagatctacatatatactagatcttcaggttggcactccaacctgcaaataggatcattagatatcaacaaccccatgaccattaaatgggcgcctcatgaagtcctgtctcgaccaggaaaccaagactgcaactggtacaacagatgtccgagagaatgcatatcaggtgtatatactgatgcatatccactatctcctgatgcagtcaatgttgctacaaccacactgtacgcaaacacatcacgtgttaatcccaccataatgtactcaaatacctcagaaattatcaacatgctaagactcaagaatgtacaactagaggcagcatacactactacatcatgtatcactcatttcgggaagggctactgcttccacattgttgaaatcaaccaagccagccttaataccttacaacctatgttgttcaagacaagtatccctaaaatatgtaaaatcacatcttgagcagatcaagacccaacactatatcaattatgtgaaaaccagatatgatgtataaaaatttaaaaacaaagcatgaatagacatttatatgacaaatagaataagaaaaacttagggttaatgcctgcctatttgtcaaatatggataaacaggagtcaactcagaattcctcagacatcttatatccagaatgtcacttgaactctccgattgtaaaaagcaagattgctcaacttcacgttttgctagatatcaatcaaccctatgatttaaaagataacagtataataaatatcaccaaatacaaaatcagaaatggaggtttatcgccccggcagatcaaaatcagatcgctaggcaaaatccttaaacaagaaattaaggatattgatcgttacacttttgaaccttatccgattttctcattagagttactcagactggatatcccagaaatatgtgacaaaataagatccattttttcagtctctgatagattaataagagaactatcatctggatttcaagaattgtggttaaatattcttagacaattaggctgtgttgaagggaaagagggatttgactcattaaaggatgtagatatcatcccagatataactgataaatataataaaaacacatggtatcgcccattcttaacatggtttagcatcaaatatgatatgagatggatgcaaaagaataagtcggggaaccatttagatgtctcaaattctcacaattttcttgactgtaaatcatatattttgattatatatagagatttagtgataataataaataaattaaaattaaccggttatgtccttacacctgaattagtattaatgtattgtgatgttgtcgaaggaagatggaatatgtcttcagctggacgactcgataaaaggtcatcaaaaataacatgtaagggggaagaattatgggagcttatcgactctttatttcccaatcttggtgaggatgtatataatattatatcactactagaacctttatcacttgctttaatacagttggatgaccctgtaactaatttaaaaggagctttcatgagacatgttttgactgagctacatacaattttaataaaagataatatatacacagattcagaagcagacagcataatggaatcattgataaagattttcagagagacatcaattgatgaaaaagcagaaattttctccttttttagaacgtttggacatcctagcttagaagcaataactgctgccgataaagtaaggacacatatgtattcctccaaaaaaatcatactaaagacactatatgagtgtcatgcaatcttctgtgcaattataataaacggatatagagaaagacacggtggtcaatggccgccatgcgaattccccaatcatgtatgtcttgaactcaagaatgcacaaggatccaactctgcaatttcgtatgaatgtgccgtagacaattatagtagttttataggatttaaatttttaaaatttattgagcctcaattagatgaagatttgacaatttatatgaaggataaggctctatcacctaggaaagcagcatgggattcagtatatcccgacagtaatttatattacaaagtccctgaatcagaagagactcgtaggttaatcgaggtttttataaatgataataattttaaccctgcggatattattaattatgtagagtcaggagaatggttaaatgacgatagcttcaacatatcttacagtctcaaagaaaaagaaattaaacaagagggtcgactctttgccaagatgacatataagatgagagcagtccaggtattagcagaaacactactagcaaaaggagtaggtgagttattcagtgaaaatgggatggtaaagggagaaattgacctactaaagagactgactacattatctgtctcaggtgttccaagatccaactcagtttacaataatcccatattacatgagaaattgatcaaaaatatgaataagtgcaattcaaatgggtattgggatgaaagaaagaaatctaaaaatgaattcaaagccgcagactcatcaaccgaggggtatgagactctgagctgttttttaaccaccgatttgaaaaaatactgtctcaactggagatttgaaagtacagcgttgttcggtcaaagatgtaatgagatattcgggtttaaaactttctttaactggatgcaccctattctagaaaaaagtacaatttatgtaggagatccttactgtccagtacctgatagaatgcacaaagaactccaagatcatgatgataccggaatctttatccataatccaagagggggaatagagggttattgccagaaattatggacactaatctctattagtgcaatccatcttgcagctgttaaagttggtgtcagagtgtcagcaatggtacaaggagacaatcaagctatagcagtgacatccagagttcctgtcacacaaacctataagcaaaaaaagactcacgtctatgaagaaatcacaagatatttcggtgccttgagagaagttatgtttgatattggacatgaattaaaattaaatgagaccattataagtagcaaaatgtttgtatacagcaaacggatatattatgatgggaaaatcctcccacagtgcctcaaagctttaacaagatgtgtattttggtcagagactcttgtagatgaaaacaggtcagcatgctcaaacattgcaacatctatagccaaagctattgagaatggatattcacctatcttaggctattgtattgctctttttaaaacttgccaacaggtatgtatatcattaggaatgaccattaatcctactattacgtcaactatcaaagatcaatattttaaagggaaaaattggttaagatgtgcaatattgatcccagctaacataggagggttcaactatatgtctacagctagatgttttgtcagaaatataggtgatccagcagttgcagctctagcagacttaaagagattcatcaaagcaggtctgttagataaacaggtattatatcgtgtgatgaatcaagaaccaggagactcaagcttcttagattgggcatcagacccttattcatgcaatctcccacactcacaaagtataacaactataatcaaaaatgtaacagctagatcagtattgcaggaatcacctaatcctctcctatcaggtctcttttcagaatcaagtagtgaagaagatctcaacttagcatcatttttgatggataggaaagccatattgcccagagtagctcacgagatcttagataactcacttacaggtgtaagagaagctatagccgggatgcttgatacaacgaaatctctagtaagagctagtgtcaggagaggaggattatcatatagtatcttaagaagacttataaattatgatctattacaatatgagaccttaacaaggacactcagaaaaccggttaaggataatatagaatatgagtatatgtgttcagtagaattggcaataggattgaggcaaaaaatgtggtttcatctaacttatggaagaccaatccacggtttagaaactccagacccgttagaattattaagaggatcatttattgaaggctcagaaatatgtaaattttgtagatcagaagggaataaccctatgtatacttggttctatcttcctgacaacatcgacttagatacacttagcaatggaagtcctgccatacgtatcccttattttggttctgctactgatgaaagatcagaggctcaactaggttatgttaagaacttaagcaagccggcaaaagcagcaataagaatcgcaatggtttacacttgggcttatggaactgatgaaatatcatggatggaagcagcacttatagctcaaaccagggctaacttaagtttagagaatttgaagttactcacccctgtatcgacttctacaaatttgtcccacagattgagagatactgctacacagatgaaattttcaagtgctactttagttcgagcgagtcgatttattaccatatctaatgataatatggcattaaaagaggcaggagagtctaaagatactaatttagtttatcaacaaattatgttaaccggattgagcttatttgaattcaatatgaggtataaacaaggatcattatctaaacctatgatattacacttacatttgaataataaatgctgtatcatagaatctcctcaagaattgaatattcctcctagatctacattggacttagagatcactcaggaaaataacaagttaatctatgatcctgatcctctcaaggacatagatctagagttatttagtaaggttagggatgtagtacacacaattgatatgaattattggtctgatgatgaaataattagagcaactagtatatgtacagctatgactattgcagacacaatgtctcaattagatagagacaatcttaaagaaatgatagcactgataaatgatgatgatataaatagtttaatcaccgaatttatggttattgatatacccttattttgttccactttcgggggtattctaatcaatcaatttgcatattcactttacgggttaaacgtcagagggagggatgaaatatggggatatgtgatacgcataattaaagacacatcacatgcagtcctaaaagtactgtccaatgcattatcacatcctaaaatattcaaacgattctgggatgcaggagttgtagagcctgtttatggacctaacttgtccaatcaagacaagatactgttagccatttcagtatgtgaatactctgttgacctcttcatgcgtgattggcaagagggcataccgcttgaaatatttatttgtgataacgacccaaatatagcagaaatgagaaaactttcatttttagctagacatctagcatacttgtgtagtttggcagagatagctaaagagggaccaaaattggaatctatgacatctctcgaacgactcgaatcattgaaagagtatctagaacttacttttttagacgatcctatattaagatatagtcaattgacaggcttagttattaagatattcccttcaacgttaacttacatcaggaaatcttcaattaaggtgttgagagtaagaggtatagggataccagaagtcttagaggactgggatcctgatgccgatagtatgctactagataatataactgctgaggttcaacacaatatacctttaaagaagaacgaaagaactcccttctgggggttaagggtatcaaaatcacaagttctgcgacttagaggttatgaagagataaaaagggaagaaagaggaagatcaggtgtaggattaactctaccttttgatgggcgatatttatcacaccaattgagacttttcgggattaatagcaccagttgtttgaaagcattggaacttacctatttactgaatcctctagtcaataaggataaagatagattatatctcggagaaggtgcaggtgcaatgctgtcttgttatgatgctacattaggaccctgcatgaactattataattcaggtgttaattcttgcgatctcaacggacaaagagaattaaatatttatccttcagaagtggcactggtagggaagaaattgaataatgtcacgagtttatgtcaaagagttaaggttttattcaatgggaatcctggatcaacttggatagggaatgatgaatgtgaaacactaatctggaatgaattacagaataattcaatagggtttattcattgtgacatggaaggtggagaacacaaatgtgatcaggtggtcttacatgaacattatagtgtgatcaggattgcataccttgttggggataaggacgttatcttagtaagcaaaattgcaccaagattaggtacagactggacaaaacaattaagtttgtatttaagatactggagagatgtcagcttaatagtgttgaaaacatctaacccagcctctacagaaatgtatctgatatcaaaagatcctaaatctgatattatagaggatagtaatacagtattggcaaaccttcttccattatctaaagaggatagtattaagatagaaaaatggattctagttgagaaagccaaagttcatgattggatagttagagaattaaaggaagggagtgcatcgtcaggtatgctaagaccttaccatcaagcattacaaatcttcggatttgagcctaatttaaacaaattatgtagagatttcttatctacactaaatatagtagacacaaaaaattgtattatcacatttgatagagtattaagagatacaatctttgagtggactcggataaaagacgcagataagaagctaagacttacaggtaaatatgatctatatcctcttagagattcaggtaagttaaaagttatttctagaaggcttgtaatatcttggatagcattgtctatgtctacaagactagtaacagggtcatttccagacattaaatttgaatcaagactccaattaggtatagtatcaatatcctctcgtgaaatcaaaaatcttagggttatatcaaagattgtcattgacaaatttgaagatattatacatagtgtgacctataggttcttgactaaagaaataaaaatattgatgaaaattttgggagcagtcaaattatttggggcaagacagagcacatctgctgatatcactaatatcgatacatcggactccatacaatgatcttatatcttctcatctttattatctaatttgtttaaagagatgagttaacaagataagaaatccctttaactgactcataaaaacatagtaagaaaaacttacaacagacaagagtattaataatatatcgatatttcttaaactcttgtctggt

SEQ ID NO: 4 - DNA, 16236 bp, Human parainfluenza virus
accaaacaagaggaaaaacttgtttggaatatataataatattaaatagtattttagggttaaagtaatactttaaagggacaagtcacagacatttgatcttagtataaatacgcgtaatggccgggctactaagtacttttgacacatttagttccaggagaagtgagagcatcaataagtctggcggaggagcaattatacctggtcaaagaagtaccgtttctgtcttcacattaggcccgagtgtgacagatgatgcagataaattattaatagcaaccactttcttagcccactcattggacacagataaacaacactctcaaagaggaggatttttagtatcactccttgcaatggcctacagtagtccggagttatatctcactacaaacggtgtcaatgctgatgtcaagtatgtgatatacaatatagagagagatcctaaaagaacaaaaacagatgggttcattgtcaaaacaagagacatggagtatgaaagaacaacagagtggttgtttggacctatgatcaacaagaacccattgttccaagggcaaagagagaatgcggatctagaggcattgcttcagacatatggatatcctgcatgtctcggagctataatagttcaagtttggatagtgttggttaaagccataacaagtagtgctggtctaagaaaaggattcttcaatagattagaagcattcagacaggatggaaccgttaaaagtgccctggtcttcacaggagacacagttgaaggtattggtgcagtgatgaggtcacaacaaagcttagtatctcttatggtagaaactctggtgactatgaacacatccaggtcagatttaactacattagagaagaacattcagattgtaggaaattacataagagatgcaggattagcatctttcatgaacaccatcaagtatggtgtagaaacgaagatggccgccctgacactatcaaatctgagaccagatataaacaaattgagaagccttgttgatatctatctatcaaagggagcccgagccccttttatatgtatactcagagacccagttcatggagactttgcccctggaaactatccagcactgtggagctacgcaatgggcgttgctgtggtacaaaacaaagctatgcaacagtatgtaactggaagaacatatttggacatggaaatgttcctacttggacaagctgtagctaaagatgctgattccaaaatcagcagtgctctggaggaagaactaggtgtgacagatacagcaaaagagagactaagacaccatctgacaaacctttcaggaggggatggtgcgtaccacaagcctacaggtggtggagctatagaagtggcaattgatcatacagacataacatttggagtcgaggacactgctgatcgggacaacaagaactggacaaatgacagcaatgaaagatggatgaatcactcgatcagcaaccacacaatcacgattcgtggtgcagaagaacttgaagaagagacaaatgatgaagacatcactgatatagaaaacaagattgcacgaaggctggccgacagaaaacagagactaagccaggcaaacaataaacgagacaccagcagtgatgctgactatgagaatgatgatgatgctacagcggctgcagggataggaggaatttaacaggatacttggacaatagaagccagatcaaaagtaagaaaaacttagggtgaatgacaattcacagatcagctcaaccagggcgcgccagcatacacgaaaccaaccttcacagtggatacctcagcatccaaaactctccttcccgagtggattaggatgcctagttttttgagagggatcctgaagccgaaggagaggcaccacgaaaacaagaatcactctcagatgtcatcggactccttgacgtcgtcttatcctacaagcccacagaaattggagaagacagaagctggctccatggtatcatcgacaacccaaaagaaaacaagccatcatgcaaagccgacgataacaacaaagacagagcaatctcaacgtcgacccaagatcatagatcaagtgagggaatctctaggagaacaagtgagtcaaaaacagagacacatgctagaatccttgatcaacaaggtatacacagggcctctaggcgaggaactagtccaaaccctctacctgagaatatgggcaatgaaagaaacaccagaatcgacgaagattctccaaatgagagaagacatcagagatcagtacttacggatgaagacagaaagatggctgagaactctaataagagggaagaagaccaagttgagggatttccagaagaggtacgaagaagtacacccttatctgatgatggagagggtagaacaaataataatggaagaagcatggaaactagcagcacacatagtacaagaataactgatgtcattaccaacccaagtttaattaagaaaaacttagggttaaagcccaacatggatcaggacgccttcttttttgagagggatcctgaagccgaaggagaggcaccacgaaaacaagaatcactctcagatgtgatcggactccttgacgtagtcttatcctacaagcccacagaaataggagaagacagaagctggctccatggtatcatcgacaacccaaaagaaaacaagccatcatgcaaagccgacgataacaacaaagacagagcaatctcaacgtcgacccaagatcatagatcaagtgaggagagtggaatctctaggagaacaagtgagtcaaaaacagagacacatgctagaatccttgatcaacaaggtatacacagggcctctaggcgaggaactagtccaaaccctctacctgagaatatgggcaatgaaagaaacaccagaatcgacgaagattctccaaatgagagaagacatcagagatcagtacttacggatgaagacagaaagatggctgagaactctaataagagggaagaagaccaagttgagggatttccagaagaggtacgaagaagtacacccttatctgatgatggagagggtagaacaaataataatggaagaagcatggaaactagcagcacacatagtacaagaataactgatgtcattaccaacccaagtccagagcttgaagatgccgttctacaaaggaacaaaagacggccgacgaccatcaagcgtaaccaaacaagatcagagagaacacagagttcagaactccacaaatcaacaagtgaaaatagctccaacctcgaagaccacaacaccaaaaccagcccaaaagttccaccgtcaaagaacgaagagtcagcagccactccaaagaacaaccacaaccacagaaaaacaagatacacaacaaacaatgcaaacaacaacacaaaaagtccaccaactcccgaacacgacgcaaccgcaaatgaagaggaaaccagcaacacatcggtcgatgagatggccaagttattagtaagtcttggtgtaatgaaatcacaacatgaatttgaattatctaggagtgcaagtcatgtatttgctaagcgcatgttaaaatctgcaaattacaaagaaatgacatttaatctctgtggtatgcttatatcagttgaaaaatcacttgagaataaagtagaagaaaatagaacattacttaaacaaattcaagaggaaataaattcatccagggatcttcacaaacggttctcggaataccaaaaagaacagaactcactcatgatggccaatctatccacactccatataattacagatagaggcgggaaaacgggaaatcccagtgatactacaaggtcaccatcagtcttcacaaaagggaaagacaataaggtcaaaaagacaaggtttgacccctctatggaagctctaggaggtcaagagttcaagcctgacttgataagagaggatgaactgagagatgacatcaaaaatccggtactagaagaaaacaacaatgagcctcaagcatccaatgcatcacgcctgattccgtccactgaaaaacacactctgcactcactcaaactagttatcgaaaacagtcctctaagcagagtagagaagaaggcttacatcaaatccctttataagtgtcggacaaaccaagaggttaaaaatgtaatggagctattcgaggaagacatagattcactaactaactaaacatgaatctacagctagcgcggccgcaaaacaattaagaaaaacttagggtcaaagaaattttgcccggagaaaggaaatggctgaaacatacaggttccccagattctcacacgaagaaaatgggacagtagaacctctccctctcaaaacaggtcctgacaaaaaagcaatccctcacatcagaatagtcaaggtaggagatcctccaaaacatggagtcaggtatcttgatgtgctactattgggattctttgaaacacctaagcaaggacctctatctggcagcatatctgatctcacagaatcaaccagttattcaatctgtggatccggatccttaccaattggcatagccaagtattacggcacagatcaagaattattaaaagcctgcattgacctcaaaataactgtacgaagaacagttagatctggagaaatgatagtatacatggtagattcgatccatgctcctctactaccatggtccagccgactgagacaagggatgatatataatgccaataaagtagctctagcacctcaatgtctcccagtcgacaaagacatcagattcagggttgtatttgtcaatggaacatcactaggtacaattacaattgctaaggtcccaaaaactcttgcagatcttgcattaccgaactcaatatcagtgaatctgctggttacacttagggcaggagtatcaacggaacaaaaaggaatcctccccgttctagacgatgatggagaaaagaagctcaacttcatggtacacctaggaatcataagaagaaaagttgggaagatatattcagttgaatactgcaaaaataaaattgagaagatgaagctaatattctctctcgggcttgtaggtggaataagtttccatgtacatgcaacaggcacattatccaaaactctaatgagccaacttgcatggaaaaaagcagtttgctatcctttaatggatgtaaatccacatatgaatctagtcatctgggcagcttcagtagaaatcacaagtgtcgatgctgtgttccaacctgcaattccgaaagaatttcgctattacccaaatgttgttgcaaaaagcatcgggaaaatcaggaggatataagtctacactcctcaataatgacacccattagctctaaatcgtaccattaatcaaatacagatcaattcgatacaatcagttcaaataagaaaaacgtagggacaaagtcctctaccaacatcaaggaagacaagagtctcaaaaagctcagcctaagcagagagaaaaacaacaacacaaagaaagaaaaggacaagatcacaaacaagaacaaaagcaaaaacaaaaacaagaacaaaaaagggaagaaaaacaaaagtatacacaaaaaccaaaaaagaaaaaaggccagagacaaaaacggaggcaagaacaaaaatttaaacaaaaacagaatttaaattcataataaacaccaagatagagacaaaaatgcaaaaatcagagatcctcttcttagtatactcaagcttgctattatcttcatcattatgtcaaattccggtagaaaaactttcaaatgtaggggttataatcaatgagggcaaattacttaaaatagcaggatcttatgaatctagatacatagtgttaagcttggtaccttcaattgacctacaagatggatgtggaacaactcaaattattcaatacaagaatttattaaatagacttctaattcctctgaaggatgccttggatcttcaggaatccctgataacaataactaatgacaccactgtgacaaatgataatccacaaactagattctttggtgctgtcattggtaccatagcactaggagtagccacagctgctcaaataactgcgggcattgcattagctgaagcacgagaagccaggaaggacatagcactaataaaagattccatagtcaagacacacaattctgtagaactcattcaaagaggtataggagaacagataattgcattaaagacattacaagattttgtaaatgacgagataagacctgcaataggagaactaaggtgtgagactacggcattgaaactagggatcaagctcacccaacactactctgaattagcaacagcattcagctccaatcttgggactataggagaaaaaagtcttaccttgcaggcattatcatctctctactctgctaatataacagaaattctaagtacaactaaaaaggataaatcagatatatatgacatcatttacactgaacaggttaagggaactgtgatagatgttgatttggaaaaatacatggttaccctcttagttaaaataccaattttatcagaaataccaggcgtgttgatatacagagcttcatctatatcttataatattgaaggagaagaatggcatgtcgcaatcccaaattacataatcaataaggcatcatccttaggaggtgcagatgtcacaaactgtatagaatcaaaattggcatatatatgtcctagagatcctacacaattaatacctgataaccaacagaagtgtatactcggggatgtatcaaagtgccctgtgactaaagtaataaacaatctagtaccaaagttcgcattcatcaatggtggtgtagtggctaattgcattgcatccacatgtacatgcgggacaaacagaataccagtgaatcaagatcgctcaagaggagttacattcttgacctataccaattgcggtttaataggtataaatggaatagaactatatgccaataaaaggggacgagacactacttgggggaatcaaatcatcaaagtgggtccagcagtctccattagacctgtagacatttctttaaatcttgcatctgccacaaatttcctagaggaatccaagacagagctcatgaaggcaagggcaatcatatcagcagttggaggatggcacaacacagagagtactcagataatcatgataataattgtgtgcatacttataataatcatatgtggtatattatactatctatacagggttagaagactattagtaatgattaattcaactcataattcacctgttaatgcttatactctggagtcaagaatgagaaatccctacatgggtaacaactccaattaaaaaatcagatcaagtacattgtagcatacatacaacaatcaaatctatccacaacttcaccaatcaggtgtacaacaagtaagaaaaacttagggttaaagacaatccagtcaacctataaggcaacagcatccgattatacaaacgatggctgaaaaagggaaaacaaatagttcatattggtctacaacccgaaatgacaattccacggtaaacacacacattaatacaccagcaggaaggacacacatctggctactgattgcaacaacaatgcatacagtattgtccttcattatcatgatcctatgcattgacctaattataaaacaagacacttgtatgaagacaaacatcatgacagtatcctccatgaacgaaagtgccaaaataatcaaagagacaatcacagaattaatcagacaagaagtaatatcaaggaccataaacatacaaagttcagtacaaagcgggatcccaatattgttaaacaagcaaagcagagatctcacacaattaatagagaagtcatgcaacagacaggaattggctcagatatgcgaaaacaccattgctattcaccatgcagacggcatatctcctctggacccacacgatttctggagatgtcccgtaggggaacccctactgagcaacaaccccaatatctcattattacctggaccaagtctactttctggatccaccacaatttcaggatgtgttagactaccttcattatcaattggtgatgcaatatatgcgtattcatcaaacttaatcactcaaggatgtgcagatatagggaagtcatatcaggttttacaattaggttacatatccttaaattcagatatgtatcctgatttaaacccggtaatttctcatacctatgacatcaacgacaacaggaaatcatgttctgtaatagctgcaggaacaaggggttatcagttatgctccttgcccactgtgaatgagactacagactactcgagtgaaggtatagaagatttagtatttgacatattagatctcaagggaaagaccaaatctcatcgatacaaaaatgaagatataacttttgaccatcctttttctgcaatgtatccgagtgtaggaagtgggataaaaattgaaaatacactcattttcctagggtacggtggcttaacaactccgctccaaggcgacactaagtgtgtgataaacagatgtaccaatgttaatcagagtgtttgcaatgatgctcttaagataacttggctaaagaaaagacaagttgtcaatgtcttaattcgtatcaataattatttatctgataggccaaagattgttgtcgagacaattccaataactcaaaattacttaggtgccgaaggtaggctacttaaactaggtaaaaagatctacatatatactagatcttcaggttggcactccaacctgcaaataggatcattagatatcaacaaccccatgaccattaaatgggcgcctcatgaagtcctgtctcgaccaggaaaccaagactgcaactggtacaacagatgtccgagagaatgcatatcaggtgtatatactgatgcatatccactatctcctgatgcagtcaatgttgctacaaccacactgtacgcaaacacatcacgtgttaatcccaccataatgtactcaaatacctcagaaattatcaacatgctaagactcaagaatgtacaactagaggcagcatacactactacatcatgtatcactcatttcgggaagggctactgcttccacattgttgaaatcaaccaagccagccttaataccttacaacctatgttgttcaagacaagtatccctaaaatatgtaaaatcacatcttgagcagatcaagacccaacactatatcaattatgtgaaaaccagatatgatgtataaaaatttaaaaacaaagcatgaatagacatttatatgacaaatagaataagaaaaacttagggttaatgcctgcctatttgtcaaatatggataaacaggagtcaactcagaattcctcagacatcttatatccagaatgtcacttgaactctccgattgtaaaaagcaagattgctcaacttcacgttttgctagatatcaatcaaccctatgatttaaaagataacagtataataaatatcaccaaatacaaaatcagaaatggaggtttatcgccccggcagatcaaaatcagatcgctaggcaaaatccttaaacaagaaattaaggatattgatcgttacacttttgaaccttatccgattttctcattagagttactcagactggatatcccagaaatatgtgacaaaataagatccattttttcagtctctgatagattaataagagaactatcatctggatttcaagaattgtggttaaatattcttagacaattaggctgtgttgaagggaaagagggatttgactcattaaaggatgtagatatcatcccagatataactgataaatataataaaaacacatggtatcgcccattcttaacatggtttagcatcaaatatgatatgagatggatgcaaaagaataagtcggggaaccatttagatgtctcaaattctcacaattttcttgactgtaaatcatatattttgattatatatagagatttagtgataataataaataaattaaaattaaccggttatgtccttacacctgaattagtattaatgtattgtgatgttgtcgaaggaagatggaatatgtcttcagctggacgactcgataaaaggtcatcaaaaataacatgtaagggggaagaattatgggagcttatcgactctttatttcccaatcttggtgaggatgtatataatattatatcactactagaacctttatcacttgctttaatacagttggatgaccctgtaactaatttaaaaggagctttcatgagacatgttttgactgagctacatacaattttaataaaagataatatatacacagattcagaagcagacagcataatggaatcattgataaagattttcagagagacatcaattgatgaaaaagcagaaattttctccttttttagaacgtttggacatcctagcttagaagcaataactgctgccgataaagtaaggacacatatgtattcctccaaaaaaatcatactaaagacactatatgagtgtcatgcaatcttctgtgcaattataataaacggatatagagaaagacacggtggtcaatggccgccatgcgaattccccaatcatgtatgtcttgaactcaagaatgcacaaggatccaactctgcaatttcgtatgaatgtgccgtagacaattatagtagttttataggatttaaatttttaaaatttattgagcctcaattagatgaagatttgacaatttatatgaaggataaggctctatcacctaggaaagcagcatgggattcagtatatcccgacagtaatttatattacaaagtccctgaatcagaagagactcgtaggttaatcgaggtttttataaatgataataattttaaccctgcggatattattaattatgtagagtcaggagaatggttaaatgacgatagcttcaacatatcttacagtctcaaagaaaaagaaattaaacaagagggtcgactctttgccaagatgacatataagatgagagcagtccaggtattagcagaaacactactagcaaaaggagtaggtgagttattcagtgaaaatgggatggtaaagggagaaattgacctactaaagagactgactacattatctgtctcaggtgttccaagatccaactcagtttacaataatcccatattacatgagaaattgatcaaaaatatgaataagtgcaattcaaatgggtattgggatgaaagaaagaaatctaaaaatgaattcaaagccgcagactcatcaaccgaggggtatgagactctgagctgttttttaaccaccgatttgaaaaaatactgtctcaactggagatttgaaagtacagcgttgttcggtcaaagatgtaatgagatattcgggtttaaaactttctttaactggatgcaccctattctagaaaaaagtacaatttatgtaggagatccttactgtccagtacctgatagaatgcacaaagaactccaagatcatgatgataccggaatctttatccataatccaagagggggaatagagggttattgccagaaattatggacactaatctctattagtgcaatccatcttgcagctgttaaagttggtgtcagagtgtcagcaatggtacaaggagacaatcaagctatagcagtgacatccagagttcctgtcacacaaacctataagcaaaaaaagactcacgtctatgaagaaatcacaagatatttcggtgccttgagagaagttatgtttgatattggacatgaattaaaattaaatgagaccattataagtagcaaaatgtttgtatacagcaaacggatatattatgatgggaaaatcctcccacagtgcctcaaagctttaacaagatgtgtattttggtcagagactcttgtagatgaaaacaggtcagcatgctcaaacattgcaacatctatagccaaagctattgagaatggatattcacctatcttaggctattgtattgctctttttaaaacttgccaacaggtatgtatatcattaggaatgaccattaatcctactattacgtcaactatcaaagatcaatattttaaagggaaaaattggttaagatgtgcaatattgatcccagctaacataggagggttcaactatatgtctacagctagatgttttgtcagaaatataggtgatccagcagttgcagctctagcagacttaaagagattcatcaaagcaggtctgttagataaacaggtattatatcgtgtgatgaatcaagaaccaggagactcaagcttcttagattgggcatcagacccttattcatgcaatctcccacactcacaaagtataacaactataatcaaaaatgtaacagctagatcagtattgcaggaatcacctaatcctctcctatcaggtctcttttcagaatcaagtagtgaagaagatctcaacttagcatcatttttgatggataggaaagccatattgcccagagtagctcacgagatcttagataactcacttacaggtgtaagagaagctatagccgggatgcttgatacaacgaaatctctagtaagagctagtgtcaggagaggaggattatcatatagtatcttaagaagacttataaattatgatctattacaatatgagaccttaacaaggacactcagaaaaccggttaaggataatatagaatatgagtatatgtgttcagtagaattggcaataggattgaggcaaaaaatgtggtttcatctaacttatggaagaccaatccacggtttagaaactccagacccgttagaattattaagaggatcatttattgaaggctcagaaatatgtaaattttgtagatcagaagggaataaccctatgtatacttggttctatcttcctgacaacatcgacttagatacacttagcaatggaagtcctgccatacgtatcccttattttggttctgctactgatgaaagatcagaggctcaactaggttatgttaagaacttaagcaagccggcaaaagcagcaataagaatcgcaatggtttacacttgggcttatggaactgatgaaatatcatggatggaagcagcacttatagctcaaaccagggctaacttaagtttagagaatttgaagttactcacccctgtatcgacttctacaaatttgtcccacagattgagagatactgctacacagatgaaattttcaagtgctactttagttcgagcgagtcgatttattaccatatctaatgataatatggcattaaaagaggcaggagagtctaaagatactaatttagtttatcaacaaattatgttaaccggattgagcttatttgaattcaatatgaggtataaacaaggatcattatctaaacctatgatattacacttacatttgaataataaatgctgtatcatagaatctcctcaagaattgaatattcctcctagatctacattggacttagagatcactcaggaaaataacaagttaatctatgatcctgatcctctcaaggacatagatctagagttatttagtaaggttagggatgtagtacacacaattgatatgaattattggtctgatgatgaaataattagagcaactagtatatgtacagctatgactattgcagacacaatgtctcaattagatagagacaatcttaaagaaatgatagcactgataaatgatgatgatataaatagtttaatcaccgaatttatggttattgatatacccttattttgttccactttcgggggtattctaatcaatcaatttgcatattcactttacgggttaaacgtcagagggagggatgaaatatggggatatgtgatacgcataattaaagacacatcacatgcagtcctaaaagtactgtccaatgcattatcacatcctaaaatattcaaacgattctgggatgcaggagttgtagagcctgtttatggacctaacttgtccaatcaagacaagatactgttagccatttcagtatgtgaatactctgttgacctcttcatgcgtgattggcaagagggcataccgcttgaaatatttatttgtgataacgacccaaatatagcagaaatgagaaaactttcatttttagctagacatctagcatacttgtgtagtttggcagagatagctaaagagggaccaaaattggaatctatgacatctctcgaacgactcgaatcattgaaagagtatctagaacttacttttttagacgatcctatattaagatatagtcaattgacaggcttagttattaagatattcccttcaacgttaacttacatcaggaaatcttcaattaaggtgttgagagtaagaggtatagggataccagaagtcttagaggactgggatcctgatgccgatagtatgctactagataatataactgctgaggttcaacacaatatacctttaaagaagaacgaaagaactcccttctgggggttaagggtatcaaaatcacaagttctgcgacttagaggttatgaagagataaaaagggaagaaagaggaagatcaggtgtaggattaactctaccttttgatgggcgatatttatcacaccaattgagacttttcgggattaatagcaccagttgtttgaaagcattggaacttacctatttactgaatcctctagtcaataaggataaagatagattatatctcggagaaggtgcaggtgcaatgctgtcttgttatgatgctacattaggaccctgcatgaactattataattcaggtgttaattcttgcgatctcaacggacaaagagaattaaatatttatccttcagaagtggcactggtagggaagaaattgaataatgtcacgagtttatgtcaaagagttaaggttttattcaatgggaatcctggatcaacttggatagggaatgatgaatgtgaaacactaatctggaatgaattacagaataattcaatagggtttattcattgtgacatggaaggtggagaacacaaatgtgatcaggtggtcttacatgaacattatagtgtgatcaggattgcataccttgttggggataaggacgttatcttagtaagcaaaattgcaccaagattaggtacagactggacaaaacaattaagtttgtatttaagatactggagagatgtcagcttaatagtgttgaaaacatctaacccagcctctacagaaatgtatctgatatcaaaagatcctaaatctgatattatagaggatagtaatacagtattggcaaaccttcttccattatctaaagaggatagtattaagatagaaaaatggattctagttgagaaagccaaagttcatgattggatagttagagaattaaaggaagggagtgcatcgtcaggtatgctaagaccttaccatcaagcattacaaatcttcggatttgagcctaatttaaacaaattatgtagagatttcttatctacactaaatatagtagacacaaaaaattgtattatcacatttgatagagtattaagagatacaatctttgagtggactcggataaaagacgcagataagaagctaagacttacaggtaaatatgatctatatcctcttagagattcaggtaagttaaaagttatttctagaaggcttgtaatatcttggatagcattgtctatgtctacaagactagtaacagggtcatttccagacattaaatttgaatcaagactccaattaggtatagtatcaatatcctctcgtgaaatcaaaaatcttagggttatatcaaagattgtcattgacaaatttgaagatattatacatagtgtgacctataggttcttgactaaagaaataaaaatattgatgaaaattttgggagcagtcaaattatttggggcaagacagagcacatctgctgatatcactaatatcgatacatcggactccatacaatgatcttatatcttctcatctttattatctaatttgtttaaagagatgagttaacaagataagaaatccctttaactgactcataaaaacatagtaagaaaaacttacaacagacaagagtattaataatatatcgatatttcttaaactcttgtctggt

SEQ ID NO: 5 - DNA, 16243 bp, Human parainfluenza virus
accaaacaagaggaaaaacttgtttggaatatataataatattaaatagtattttagggttaaagtaatactttaaagggacaagtcacagacatttgatcttagtataaatacgcgtaatggccgggctactaagtacttttgacacatttagttccaggagaagtgagagcatcaataagtctggcggaggagcaattatacctggtcaaagaagtaccgtttctgtcttcacattaggcccgagtgtgacagatgatgcagataaattattaatagcaaccactttcttagcccactcattggacacagataaacaacactctcaaagaggaggatttttagtatcactccttgcaatggcctacagtagtccggagttatatctcactacaaacggtgtcaatgctgatgtcaagtatgtgatatacaatatagagagagatcctaaaagaacaaaaacagatgggttcattgtcaaaacaagagacatggagtatgaaagaacaacagagtggttgtttggacctatgatcaacaagaacccattgttccaagggcaaagagagaatgcggatctagaggcattgcttcagacatatggatatcctgcatgtctcggagctataatagttcaagtttggatagtgttggttaaagccataacaagtagtgctggtctaagaaaaggattcttcaatagattagaagcattcagacaggatggaaccgttaaaagtgccctggtcttcacaggagacacagttgaaggtattggtgcagtgatgaggtcacaacaaagcttagtatctcttatggtagaaactctggtgactatgaacacatccaggtcagatttaactacattagagaagaacattcagattgtaggaaattacataagagatgcaggattagcatctttcatgaacaccatcaagtatggtgtagaaacgaagatggccgccctgacactatcaaatctgagaccagatataaacaaattgagaagccttgttgatatctatctatcaaagggagcccgagccccttttatatgtatactcagagacccagttcatggagactttgcccctggaaactatccagcactgtggagctacgcaatgggcgttgctgtggtacaaaacaaagctatgcaacagtatgtaactggaagaacatatttggacatggaaatgttcctacttggacaagctgtagctaaagatgctgattccaaaatcagcagtgctctggaggaagaactaggtgtgacagatacagcaaaagagagactaagacaccatctgacaaacctttcaggaggggatggtgcgtaccacaagcctacaggtggtggagctatagaagtggcaattgatcatacagacataacatttggagtcgaggacactgctgatcgggacaacaagaactggacaaatgacagcaatgaaagatggatgaatcactcgatcagcaaccacacaatcacgattcgtggtgcagaagaacttgaagaagagacaaatgatgaagacatcactgatatagaaaacaagattgcacgaaggctggccgacagaaaacagagactaagccaggcaaacaataaacgagacaccagcagtgatgctgactatgagaatgatgatgatgctacagcggctgcagggataggaggaatttaacaggatacttggacaatagaagccagatcaaaagtaagaaaaacttagggtgaatgacaattcacagatcagctcaaccagggcgcgccagcatacacgaaaccaaccttcacataggatacctcagcatccaaaactctccttcccgagtggattaggacgcctagttttttgagagggatcctgaagccgaaggagaggcaccacgaaaacaagaatcactctcagatgtgatcggactccttgacgtagtcttatcctacaagcccacagaaatagtgagaagacagaagctggctcctaggtatcatcgacaacccaaaagaaaacaagccatcatgcaaagccgacgataacaacaaagacagagcaatctcaacgtcgacccaagatcatagatcaagtgaggagagtggaatctctaggagaacaagtgagtcaaaaacagagacacatgctagaatccttgatcaacaaggtatacacagggcctctaggcgagtaactagtccaaaccctctacctgagaattagggcatagaaagaaacaccagaatcgacgaagattctccaaatgagagaagacatcagagatcagtacttacggatgaagacagaaagatggctgagaactctaataagagggaagaagaccaagttgagggatttccagtagaggtacgaagaagtacacccttatctgatgatggagagggttgaacaaataataatggaagaagcatggaaactagcagcacacatagtacaagaataactgatgtcattaccaacccaagtttaattaagaaaaacttagggttaaagcccaacatggatcaggacgccttcttttttgagagggatcctgaagccgaaggagaggcaccacgaaaacaagaatcactctcagatgtgatcggactccttgacgtagtcttatcctacaagcccacagaaataggagaagacagaagctggctccatggtatcatcgacaacccaaaagaaaacaagccatcatgcaaagccgacgataacaacaaagacagagcaatctcaacgtcgacccaagatcatagatcaagtgaggagagtggaatctctaggagaacaagtgagtcaaaaacagagacacatgctagaatccttgatcaacaaggtatacacagggcctctaggcgaggaactagtccaaaccctctacctgagaatatgggcaatgaaagaaacaccagaatcgacgaagattctccaaatgagagaagacatcagagatcagtacttacggatgaagacagaaagatggctgagaactctaataagagggaagaagaccaagttgagggatttccagaagaggtacgaagaagtacacccttatctgatgatggagagggtagaacaaataataatggaagaagcatggaaactagcagcacacatagtacaagaataactgatgtcattaccaacccaagtccagagcttgaagatgccgttctacaaaggaacaaaagacggccgacgaccatcaagcgtaaccaaacaagatcagagagaacacagagttcagaactccacaaatcaacaagtgaaaatagctccaacctcgaagaccacaacaccaaaaccagcccaaaagttccaccgtcaaagaacgaagagtcagcagccactccaaagaacaaccacaaccacagaaaaacaagatacacaacaaacaatgcaaacaacaacacaaaaagtccaccaactcccgaacacgacgcaaccgcaaatgaagaggaaaccagcaacacatcggtcgatgagatggccaagttattagtaagtcttggtgtaatgaaatcacaacatgaatttgaattatctaggagtgcaagtcatgtatttgctaagcgcatgttaaaatctgcaaattacaaagaaatgacatttaatctctgtggtatgcttatatcagttgaaaaatcacttgagaataaagtagaagaaaatagaacattacttaaacaaattcaagaggaaataaattcatccagggatcttcacaaacggttctcggaataccaaaaagaacagaactcactcatgatggccaatctatccacactccatataattacagatagaggcgggaaaacgggaaatcccagtgatactacaaggtcaccatcagtcttcacaaaagggaaagacaataaggtcaaaaagacaaggtttgacccctctatggaagctctaggaggtcaagagttcaagcctgacttgataagagaggatgaactgagagatgacatcaaaaatccggtactagaagaaaacaacaatgagcctcaagcatccaatgcatcacgcctgattccgtccactgaaaaacacactctgcactcactcaaactagttatcgaaaacagtcctctaagcagagtagagaagaaggcttacatcaaatccctttataagtgtcggacaaaccaagaggttaaaaatgtaatggagctattcgaggaagacatagattcactaactaactaaacatgaatctacagctagcgcggccgcaaaacaattaagaaaaacttagggtcaaagaaattttgcccggagaaaggaaatggctgaaacatacaggttccccagattctcacacgaagaaaatgggacagtagaacctctccctctcaaaacaggtcctgacaaaaaagcaatccctcacatcagaatagtcaaggtaggagatcctccaaaacatggagtcaggtatcttgatgtgctactattgggattctttgaaacacctaagcaaggacctctatctggcagcatatctgatctcacagaatcaaccagttattcaatctgtggatccggatccttaccaattggcatagccaagtattacggcacagatcaagaattattaaaagcctgcattgacctcaaaataactgtacgaagaacagttagatctggagaaatgatagtatacatggtagattcgatccatgctcctctactaccatggtccagccgactgagacaagggatgatatataatgccaataaagtagctctagcacctcaatgtctcccagtcgacaaagacatcagattcagggttgtatttgtcaatggaacatcactaggtacaattacaattgctaaggtcccaaaaactcttgcagatcttgcattaccgaactcaatatcagtgaatctgctggttacacttagggcaggagtatcaacggaacaaaaaggaatcctccccgttctagacgatgatggagaaaagaagctcaacttcatggtacacctaggaatcataagaagaaaagttgggaagatatattcagttgaatactgcaaaaataaaattgagaagatgaagctaatattctctctcgggcttgtaggtggaataagtttccatgtacatgcaacaggcacattatccaaaactctaatgagccaacttgcatggaaaaaagcagtttgctatcctttaatggatgtaaatccacatatgaatctagtcatctgggcagcttcagtagaaatcacaagtgtagatgctgtgttccaacctgcaattccgaaagaatttcgctattacccaaatgttgttgcaaaaagcatcgggaaaatcaggaggatataagtctacactcctcaataatgacacccattagctctaaatcgtaccattaatcaaatacagatcaattcgatacaatcagttcaaataagaaaaacgtagggacaaagtcctctaccaacatcaaggaagacaagagtctcaaaaagctcagcctaagcagagagaaaaacaacaacacaaagaaagaaaaggacaagatcacaaacaagaacaaaagcaaaaacaaaaacaagaacaaaaaagggaagaaaaacaaaagtatacacaaaaaccaaaaaagaaaaaaggccagagacaaaaacggaggcaagaacaaaaatttaaacaaaaacagaatttaaattcataataaacaccaagatagagacaaaaatgcaaaaatcagagatcctcttcttagtatactcaagcttgctattatcttcatcattatgtcaaattccggtagaaaaactttcaaatgtaggggttataatcaatgagggcaaattacttaaaatagcaggatcttatgaatctagatacatagtgttaagcttggtaccttcaattgacctacaagatggatgtggaacaactcaaattattcaatacaagaatttattaaatagacttctaattcctctgaaggatgccttggatcttcaggaatccctgataacaataactaatgacaccactgtgacaaatgataatccacaaactagattctttggtgctgtcattggtaccatagcactaggagtagccacagctgctcaaataactgcgggcattgcattagctgaagcacgagaagccaggaaggacatagcactaataaaagattccatagtcaagacacacaattctgtagaactcattcaaagaggtataggagaacagataattgcattaaagacattacaagattttgtaaatgacgagataagacctgcaataggagaactaaggtgtgagactacggcattgaaactagggatcaagctcacccaacactactctgaattagcaacagcattcagctccaatcttgggactataggagaaaaaagtcttaccttgcaggcattatcatctctctactctgctaatataacagaaattctaagtacaactaaaaaggataaatcagatatatatgacatcatttacactgaacaggttaagggaactgtgatagatgttgatttggaaaaatacatggttaccctcttagttaaaataccaattttatcagaaataccaggcgtgttgatatacagagcttcatctatatcttataatattgaaggagaagaatggcatgtcgcaatcccaaattacataatcaataaggcatcatccttaggaggtgcagatgtcacaaactgtatagaatcaaaattggcatatatatgtcctagagatcctacacaattaatacctgataaccaacagaagtgtatactcggggatgtatcaaagtgccctgtgactaaagtaataaacaatctagtaccaaagttcgcattcatcaatggtggtgtagtggctaattgcattgcatccacatgtacatgcgggacaaacagaataccagtgaatcaagatcgctcaagaggagttacattcttgacctataccaattgcggtttaataggtataaatggaatagaactatatgccaataaaaggggacgagacactacttgggggaatcaaatcatcaaagtgggtccagcagtctccattagacctgtagacatttctttaaatcttgcatctgccacaaatttcctagaggaatccaagacagagctcatgaaggcaagggcaatcatatcagcagttggaggatggcacaacacagagagtactcagataatcatgataataattgtgtgcatacttataataatcatatgtggtatattatactatctatacagggttagaagactattagtaatgattaattcaactcataattcacctgttaatgcttatactctggagtcaagaatgagaaatccctacatgggtaacaactccaattaaaaaatcagatcaagtacattgtagcatacatacaacaatcaaatctatccacaacttcaccaatcaggtgtacaacaagtaagaaaaacttagggttaaagacaatccagtcaacctataaggcaacagcatccgattatacaaacgatggctgaaaaagggaaaacaaatagttcatattggtctacaacccgaaatgacaattccacggtaaacacacacattaatacaccagcaggaaggacacacatctggctactgattgcaacaacaatgcatacagtattgtccttcattatcatgatcctatgcattgacctaattataaaacaagacacttgtatgaagacaaacatcatgacagtatcctccatgaacgaaagtgccaaaataatcaaagagacaatcacagaattaatcagacaagaagtaatatcaaggaccataaacatacaaagttcagtacaaagcgggatcccaatattgttaaacaagcaaagcagagatctcacacaattaatagagaagtcatgcaacagacaggaattggctcagatatgcgaaaacaccattgctattcaccatgcagacggcatatctcctctggacccacacgatttctggagatgtcccgtaggggaacccctactgagcaacaaccccaatatctcattattacctggaccaagtctactttctggatccaccacaatttcaggatgtgttagactaccttcattatcaattggtgatgcaatatatgcgtattcatcaaacttaatcactcaaggatgtgcagatatagggaagtcatatcaggttttacaattaggttacatatccttaaattcagatatgtatcctgatttaaacccggtaatttctcatacctatgacatcaacgacaacaggaaatcatgttctgtaatagctgcaggaacaaggggttatcagttatgctccttgcccactgtgaatgagactacagactactcgagtgaaggtatagaagatttagtatttgacatattagatctcaagggaaagaccaaatctcatcgatacaaaaatgaagatataacttttgaccatcctttttctgcaatgtatccgagtgtaggaagtgggataaaaattgaaaatacactcattttcctagggtacggtggcttaacaactccgctccaaggcgacactaagtgtgtgataaacagatgtaccaatgttaatcagagtgtttgcaatgatgctcttaagataacttggctaaagaaaagacaagttgtcaatgtcttaattcgtatcaataattatttatctgataggccaaagattgttgtcgagacaattccaataactcaaaattacttaggtgccgaaggtaggctacttaaactaggtaaaaagatctacatatatactagatcttcaggttggcactccaacctgcaaataggatcattagatatcaacaaccccatgaccattaaatgggcgcctcatgaagtcctgtctcgaccaggaaaccaagactgcaactggtacaacagatgtccgagagaatgcatatcaggtgtatatactgatgcatatccactatctcctgatgcagtcaatgttgctacaaccacactgtacgcaaacacatcacgtgttaatcccaccataatgtactcaaatacctcagaaattatcaacatgctaagactcaagaatgtacaactagaggcagcatacactactacatcatgtatcactcatttcgggaagggctactgcttccacattgttgaaatcaaccaagccagccttaataccttacaacctatgttgttcaagacaagtatccctaaaatatgtaaaatcacatcttgagcagatcaagacccaacactatatcaattatgtgaaaaccagatatgatgtataaaaatttaaaaacaaagcatgaatagacatttatatgacaaatagaataagaaaaacttagggttaatgcctgcctatttgtcaaatatggataaacaggagtcaactcagaattcctcagacatcttatatccagaatgtcacttgaactctccgattgtaaaaagcaagattgctcaacttcacgttttgctagatatcaatcaaccctatgatttaaaagataacagtataataaatatcaccaaatacaaaatcagaaatggaggtttatcgccccggcagatcaaaatcagatcgctaggcaaaatccttaaacaagaaattaaggatattgatcgttacacttttgaaccttatccgattttctcattagagttactcagactggatatcccagaaatatgtgacaaaataagatccattttttcagtctctgatagattaataagagaactatcatctggatttcaagaattgtggttaaatattcttagacaattaggctgtgttgaagggaaagagggatttgactcattaaaggatgtagatatcatcccagatataactgataaatataataaaaacacatggtatcgcccattcttaacatggtttagcatcaaatatgatatgagatggatgcaaaagaataagtcggggaaccatttagatgtctcaaattctcacaattttcttgactgtaaatcatatattttgattatatatagagatttagtgataataataaataaattaaaattaaccggttatgtccttacacctgaattagtattaatgtattgtgatgttgtcgaaggaagatggaatatgtcttcagctggacgactcgataaaaggtcatcaaaaataacatgtaagggggaagaattatgggagcttatcgactctttatttcccaatcttggtgaggatgtatataatattatatcactactagaacctttatcacttgctttaatacagttggatgaccctgtaactaatttaaaaggagctttcatgagacatgttttgactgagctacatacaattttaataaaagataatatatacacagattcagaagcagacagcataatggaatcattgataaagattttcagagagacatcaattgatgaaaaagcagaaattttctccttttttagaacgtttggacatcctagcttagaagcaataactgctgccgataaagtaaggacacatatgtattcctccaaaaaaatcatactaaagacactatatgagtgtcatgcaatcttctgtgcaattataataaacggatatagagaaagacacggtggtcaatggccgccatgcgaattccccaatcatgtatgtcttgaactcaagaatgcacaaggatccaactctgcaatttcgtatgaatgtgccgtagacaattatagtagttttataggatttaaatttttaaaatttattgagcctcaattagatgaagatttgacaatttatatgaaggataaggctctatcacctaggaaagcagcatgggattcagtatatcccgacagtaatttatattacaaagtccctgaatcagaagagactcgtaggttaatcgaggtttttataaatgataataattttaaccctgcggatattattaattatgtagagtcaggagaatggttaaatgacgatagcttcaacatatcttacagtctcaaagaaaaagaaattaaacaagagggtcgactctttgccaagatgacatataagatgagagcagtccaggtattagcagaaacactactagcaaaaggagtaggtgagttattcagtgaaaatgggatggtaaagggagaaattgacctactaaagagactgactacattatctgtctcaggtgttccaagatccaactcagtttacaataatcccatattacatgagaaattgatcaaaaatatgaataagtgcaattcaaatgggtattgggatgaaagaaagaaatctaaaaatgaattcaaagctgcagactcatcaaccgaggggtatgagactctgagctgttttttaaccaccgatttgaaaaaatactgtctcaactggagatttgaaagtacagcgttgttcggtcaaagatgtaatgagatattcgggtttaaaactttctttaactggatgcaccctattctagaaaaaagtacaatttatgtaggagatccttactgtccagtacctgatagaatgcacaaagaactccaagatcatgatgataccggaatctttatccataatccaagagggggaatagagggttattgccagaaattatggacactaatctctattagtgcaatccatcttgcagctgttaaagttggtgtcagagtgtcagcaatggtacaaggagacaatcaagctatagcagtgacatccagagttcctgtcacacaaacctataagcaaaaaaagactcacgtctatgaagaaatcacaagatatttcggtgccttgagagaagttatgtttgatattggacatgaattaaaattaaatgagaccattataagtagcaaaatgtttgtatacagcaaacggatatattatgatgggaaaatcctcccacagtgcctcaaagctttaacaagatgtgtattttggtcagagactcttgtagatgaaaacaggtcagcatgctcaaacattgcaacatctatagccaaagctattgagaatggatattcacctatcttaggctattgtattgctctttttaaaacttgccaacaggtatgtatatcattaggaatgaccattaatcctactattacgtcaactatcaaagatcaatattttaaagggaaaaattggttaagatgtgcaatattgatcccagctaacataggagggttcaactatatgtctacagctagatgttttgtcagaaatataggtgatccagcagttgcagctctagcagacttaaagagattcatcaaagcaggtctgttagataaacaggtattatatcgtgtgatgaatcaagaaccaggagactcaagcttcttagattgggcatcagacccttattcatgcaatctcccacactcacaaagtataacaactataatcaaaaatgtaacagctagatcagtattgcaggaatcacctaatcctctcctatcaggtctcttttcagaatcaagtagtgaagaagatctcaacttagcatcatttttgatggataggaaagccatattgcccagagtagctcacgagatcttagataactcacttacaggtgtaagagaagctatagccgggatgcttgatacaacgaaatctctagtaagagctagtgtcaggagaggaggattatcatatagtatcttaagaagacttataaattatgatctattacaatatgagaccttaacaaggacactcagaaaaccggttaaggataatatagaatatgagtatatgtgttcagtagaattggcaataggattgaggcaaaaaatgtggtttcatctaacttatggaagaccaatccacggtttagaaactccagacccgttagaattattaagaggatcatttattgaaggctcagaaatatgtaaattttgtagatcagaagggaataaccctatgtatacttggttctatcttcctgacaacatcgacttagatacacttagcaatggaagtcctgccatacgtatcccttattttggttctgctactgatgaaagatcagaggctcaactaggttatgttaagaacttaagcaagccggcaaaagcagcaataagaatcgcaatggtttacacttgggcttatggaactgatgaaatatcatggatggaagcagcacttatagctcaaaccagggctaacttaagtttagagaatttgaagttactcacccctgtatcgacttctacaaatttgtcccacagattgagagatactgctacacagatgaaattttcaagtgctactttagttcgagcgagtcgatttattaccatatctaatgataatatggcattaaaagaggcaggagagtctaaagatactaatttagtttatcaacaaattatgttaaccggattgagcttatttgaattcaatatgaggtataaacaaggatcattatctaaacctatgatattacacttacatttgaataataaatgctgtatcatagaatctcctcaagaattgaatattcctcctagatctacattggacttagagatcactcaggaaaataacaagttaatctatgatcctgatcctctcaaggacatagatctagagttatttagtaaggttagggatgtagtacacacaattgatatgaattattggtctgatgatgaaataattagagcaactagtatatgtacagctatgactattgcagacacaatgtctcaattagatagagacaatcttaaagaaatgatagcactgataaatgatgatgatataaatagtttaatcaccgaatttatggttattgatatacccttattttgttccactttcgggggtattctaatcaatcaatttgcatattcactttacgggttaaacgtcagagggagggatgaaatatggggatatgtgatacgcataattaaagacacatcacatgcagtcctaaaagtactgtccaatgcattatcacatcctaaaatattcaaacgattctgggatgcaggagttgtagagcctgtttatggacctaacttgtccaatcaagacaagatactgttagccatttcagtatgtgaatactctgttgacctcttcatgcgtgattggcaagagggcataccgcttgaaatatttatttgtgataacgacccaaatatagcagaaatgagaaaactttcatttttagctagacatctagcatacttgtgtagtttggcagagatagctaaagagggaccaaaattggaatctatgacatctctcgaacgactcgaatcattgaaagagtatctagaacttacttttttagacgatcctatattaagatatagtcaattgacaggcttagttattaagatattcccttcaacgttaacttacatcaggaaatcttcaattaaggtgttgagagtaagaggtatagggataccagaagtcttagaggactgggatcctgatgccgatagtatgctactagataatataactgctgaggttcaacacaatatacctttaaagaagaacgaaagaactcccttctgggggttaagggtatcaaaatcacaagttctgcgacttagaggttatgaagagataaaaagggaagaaagaggaagatcaggtgtaggattaactctaccttttgatgggcgatatttatcacaccaattgagacttttcgggattaatagcaccagttgtttgaaagcattggaacttacctatttactgaatcctctagtcaataaggataaagatagattatatctcggagaaggtgcaggtgcaatgctgtcttgttatgatgctacattaggaccctgcatgaactattataattcaggtgttaattcttgtgatctcaacggacaaagagaattaaatatttatccttcagaagtggcactggtagggaagaaattgaataatgtcacgagtttatgtcaaagagttaaggttttattcaatgggaatcctggatcaacttggatagggaatgatgaatgtgaaacactaatctggaatgaattacagaataattcaatagggtttattcattgtgacatggaaggtggagaacacaaatgtgatcaggtggtcttacatgaacattatagtgtgatcaggattgcataccttgttggggataaggacgttatcttagtaagcaaaattgcaccaagattaggtacagactggacaaaacaattaagtttgtatttaagatactggagagatgtcagcttaatagtgttgaaaacatctaacccagcctctacagaaatgtatctgatatcaaaagatcctaaatctgatattatagaggatagtaatacagtattggcaaaccttcttccattatctaaagaggatagtattaagatagaaaaatggattctagttgagaaagccaaagttcatgattggatagttagagaattaaaggaagggagtgcatcgtcaggtatgctaagaccttaccatcaagcattacaaatcttcggatttgagcctaatttaaacaaattatgtagagatttcttatctacactaaatatagtagacacaaaaaattgtattatcacatttgatagagtattaagagatacaatctttgagtggactcggataaaagacgcagataagaagctaagacttacaggtaaatatgatctatatcctcttagagattcaggtaagttaaaagttatttctagaaggcttgtaatatcttggatagcattgtctatgtctacaagactagtaacagggtcatttccagacattaaatttgaatcaagactccaattaggtatagtatcaatatcctctcgtgaaatcaaaaatcttagggttatatcaaagattgtcattgacaaatttgaagatattatacatagtgtgacctataggttcttgactaaagaaataaaaatattgatgaaaattttgggagcagtcaaattatttggggcaagacagagcacatctgctgatatcactaatatcgatacatcggactccatacaatgatcttatatcttctcatctttattatctaatttgtttaaagagatgagttaacaagataagaaatccctttaactgactcataaaaacatagtaagaaaaacttacaacagacaagagtattaataatatatcgatatttcttaaactcttgtctggt

SEQ ID NO: 6 - DNA, 15474 bp, Human parainfluenza virus
accaaacaagaggaaaaacttgtttggaatatataataatattaaatagtattttagggttaaagtaatactttaaagggacaagtcacagacatttgatcttagtataaatacgcgtaatggccgggctactaagtacttttgacacatttagttccaggagaagtgagagcatcaataagtctggcggaggagcaattatacctggtcaaagaagtaccgtttctgtcttcacattaggcccgagtgtgacagatgatgcagataaattattaatagcaaccactttcttagcccactcattggacacagataaacaacactctcaaagaggaggatttttagtatcactccttgcaatggcctacagtagtccggagttatatctcactacaaacggtgtcaatgctgatgtcaagtatgtgatatacaatatagagagagatcctaaaagaacaaaaacagatgggttcattgtcaaaacaagagacatggagtatgaaagaacaacagagtggttgtttggacctatgatcaacaagaacccattgttccaagggcaaagagagaatgcggatctagaggcattgcttcagacatatggatatcctgcatgtctcggagctataatagttcaagtttggatagtgttggttaaagccataacaagtagtgctggtctaagaaaaggattcttcaatagattagaagcattcagacaggatggaaccgttaaaagtgccctggtcttcacaggagacacagttgaaggtattggtgcagtgatgaggtcacaacaaagcttagtatctcttatggtagaaactctggtgactatgaacacatccaggtcagatttaactacattagagaagaacattcagattgtaggaaattacataagagatgcaggattagcatctttcatgaacaccatcaagtatggtgtagaaacgaagatggccgccctgacactatcaaatctgagaccagatataaacaaattgagaagccttgttgatatctatctatcaaagggagcccgagccccttttatatgtatactcagagacccagttcatggagactttgcccctggaaactatccagcactgtggagctacgcaatgggcgttgctgtggtacaaaacaaagctatgcaacagtatgtaactggaagaacatatttggacatggaaatgttcctacttggacaagctgtagctaaagatgctgattccaaaatcagcagtgctctggaggaagaactaggtgtgacagatacagcaaaagagagactaagacaccatctgacaaacctttcaggaggggatggtgcgtaccacaagcctacaggtggtggagctatagaagtggcaattgatcatacagacataacatttggagtcgaggacactgctgatcgggacaacaagaactggacaaatgacagcaatgaaagatggatgaatcactcgatcagcaaccacacaatcacgattcgtggtgcagaagaacttgaagaagagacaaatgatgaagacatcactgatatagaaaacaagattgcacgaaggctggccgacagaaaacagagactaagccaggcaaacaataaacgagacaccagcagtgatgctgactatgagaatgatgatgatgctacagcggctgcagggataggaggaatttaacaggatacttggacaatagaagccagatcaaaagtaagaaaaacttagggttaaagcccaacatggatcaggacgccttcttttttgagagggatcctgaagccgaaggagaggcaccacgaaaacaagaatcactctcagatgtgatcggactccttgacgtagtcttatcctacaagcccacagaaataggagaagacagaagctggctccatggtatcatcgacaacccaaaagaaaacaagccatcatgcaaagccgacgataacaacaaagacagagcaatctcaacgtcgacccaagatcatagatcaagtgaggagagtggaatctctaggagaacaagtgagtcaaaaacagagacacatgctagaatccttgatcaacaaggtatacacagggcctctaggcgaggaactagtccaaaccctctacctgagaatatgggcaatgaaagaaacaccagaatcgacgaagattctccaaatgagagaagacatcagagatcagtacttacggatgaagacagaaagatggctgagaactctaataagagggaagaagaccaagttgagggatttccagaagaggtacgaagaagtacacccttatctgatgatggagagggtagaacaaataataatggaagaagcatggaaactagcagcacacatagtacaagaataactgatgtcattaccaacccaagtccagagcttgaagatgccgttctacaaaggaacaaaagacggccgacgaccatcaagcgtaaccaaacaagatcagagagaacacagagttcagaactccacaaatcaacaagtgaaaatagctccaacctcgaagaccacaacaccaaaaccagcccaaaagttccaccgtcaaagaacgaagagtcagcagccactccaaagaacaaccacaaccacagaaaaacaagatacacaacaaacaatgcaaacaacaacacaaaaagtccaccaactcccgaacacgacgcaaccgcaaatgaagaggaaaccagcaacacatcggtcgatgagatggccaagttattagtaagtcttggtgtaatgaaatcacaacatgaatttgaattatctaggagtgcaagtcatgtatttgctaagcgcatgttaaaatctgcaaattacaaagaaatgacatttaatctctgtggtatgcttatatcagttgaaaaatcacttgagaataaagtagaagaaaatagaacattacttaaacaaattcaagaggaaataaattcatccagggatcttcacaaacggttctcggaataccaaaaagaacagaactcactcatgatggccaatctatccacactccatataattacagatagaggcgggaaaacgggaaatcccagtgatactacaaggtcaccatcagtcttcacaaaagggaaagacaataaggtcaaaaagacaaggtttgacccctctatggaagctctaggaggtcaagagttcaagcctgacttgataagagaggatgaactgagagatgacatcaaaaatccggtactagaagaaaacaacaatgagcctcaagcatccaatgcatcacgcctgattccgtccactgaaaaacacactctgcactcactcaaactagttatcgaaaacagtcctctaagcagagtagagaagaaggcttacatcaaatccctttataagtgtcggacaaaccaagaggttaaaaatgtaatggagctattcgaggaagacatagattcactaactaactaaacatgaatctacagctagcgcggccgcaaaacaattaagaaaaacttagggtcaaagaaattttgcccggagaaaggaaatggctgaaacatacaggttccccagattctcacacgaagaaaatgggacagtagaacctctccctctcaaaacaggtcctgacaaaaaagcaatccctcacatcagaatagtcaaggtaggagatcctccaaaacatggagtcaggtatcttgatgtgctactattgggattctttgaaacacctaagcaaggacctctatctggcagcatatctgatctcacagaatcaaccagttattcaatctgtggatccggatccttaccaattggcatagccaagtattacggcacagatcaagaattattaaaagcctgcattgacctcaaaataactgtacgaagaacagttagatctggagaaatgatagtatacatggtagattcgatccatgctcctctactaccatggtccagccgactgagacaagggatgatatataatgccaataaagtagctctagcacctcaatgtctcccagtcgacaaagacatcagattcagggttgtatttgtcaatggaacatcactaggtacaattacaattgctaaggtcccaaaaactcttgcagatcttgcattaccgaactcaatatcagtgaatctgctggttacacttagggcaggagtatcaacggaacaaaaaggaatcctccccgttctagacgatgatggagaaaagaagctcaacttcatggtacacctaggaatcataagaagaaaagttgggaagatatattcagttgaatactgcaaaaataaaattgagaagatgaagctaatattctctctcgggcttgtaggtggaataagtttccatgtacatgcaacaggcacattatccaaaactctaatgagccaacttgcatggaaaaaagcagtttgctatcctttaatggatgtaaatccacatatgaatctagtcatctgggcagcttcagtagaaatcacaagtgtagatgctgtgttccaacctgcaattccgaaagaatttcgctattacccaaatgttgttgcaaaaagcatcgggaaaatcaggaggatataagtctacactcctcaataatgacacccattagctctaaatcgtaccattaatcaaatacagatcaattcgatacaatcagttcaaataagaaaaacgtagggacaaagtcctctaccaacatcaaggaagacaagagtctcaaaaagctcagcctaagcagagagaaaaacaacaacacaaagaaagaaaaggacaagatcacaaacaagaacaaaagcaaaaacaaaaacaagaacaaaaaagggaagaaaaacaaaagtatacacaaaaaccaaaaaagaaaaaaggccagagacaaaaacggaggcaagaacaaaaatttaaacaaaaacagaatttaaattcataataaacaccaagatagagacaaaaatgcaaaaatcagagatcctcttcttagtatactcaagcttgctattatcttcatcattatgtcaaattccggtagaaaaactttcaaatgtaggggttataatcaatgagggcaaattacttaaaatagcaggatcttatgaatctagatacatagtgttaagcttggtaccttcaattgacctacaagatggatgtggaacaactcaaattattcaatacaagaatttattaaatagacttctaattcctctgaaggatgccttggatcttcaggaatccctgataacaataactaatgacaccactgtgacaaatgataatccacaaactagattctttggtgctgtcattggtaccatagcactaggagtagccacagctgctcaaataactgcgggcattgcattagctgaagcacgagaagccaggaaggacatagcactaataaaagattccatagtcaagacacacaattctgtagaactcattcaaagaggtataggagaacagataattgcattaaagacattacaagattttgtaaatgacgagataagacctgcaataggagaactaaggtgtgagactacggcattgaaactagggatcaagctcacccaacactactctgaattagcaacagcattcagctccaatcttgggactataggagaaaaaagtcttaccttgcaggcattatcatctctctactctgctaatataacagaaattctaagtacaactaaaaaggataaatcagatatatatgacatcatttacactgaacaggttaagggaactgtgatagatgttgatttggaaaaatacatggttaccctcttagttaaaataccaattttatcagaaataccaggcgtgttgatatacagagcttcatctatatcttataatattgaaggagaagaatggcatgtcgcaatcccaaattacataatcaataaggcatcatccttaggaggtgcagatgtcacaaactgtatagaatcaaaattggcatatatatgtcctagagatcctacacaattaatacctgataaccaacagaagtgtatactcggggatgtatcaaagtgccctgtgactaaagtaataaacaatctagtaccaaagttcgcattcatcaatggtggtgtagtggctaattgcattgcatccacatgtacatgcgggacaaacagaataccagtgaatcaagatcgctcaagaggagttacattcttgacctataccaattgcggtttaataggtataaatggaatagaactatatgccaataaaaggggacgagacactacttgggggaatcaaatcatcaaagtgggtccagcagtctccattagacctgtagacatttctttaaatcttgcatctgccacaaatttcctagaggaatccaagacagagctcatgaaggcaagggcaatcatatcagcagttggaggatggcacaacacagagagtactcagataatcatgataataattgtgtgcatacttataataatcatatgtggtatattatactatctatacagggttagaagactattagtaatgattaattcaactcataattcacctgttaatgcttatactctggagtcaagaatgagaaatccctacatgggtaacaactccaattaaaaaatcagatcaagtacattgtagcatacatacaacaatcaaatctatccacaacttcaccaatcaggtgtacaacaagtaagaaaaacttagggttaaagacaatccagtcaacctataaggcaacagcatccgattatacaaacgatggctgaaaaagggaaaacaaatagttcatattggtctacaacccgaaatgacaattccacggtaaacacacacattaatacaccagcaggaaggacacacatctggctactgattgcaacaacaatgcatacagtattgtccttcattatcatgatcctatgcattgacctaattataaaacaagacacttgtatgaagacaaacatcatgacagtatcctccatgaacgaaagtgccaaaataatcaaagagacaatcacagaattaatcagacaagaagtaatatcaaggaccataaacatacaaagttcagtacaaagcgggatcccaatattgttaaacaagcaaagcagagatctcacacaattaatagagaagtcatgcaacagacaggaattggctcagatatgcgaaaacaccattgctattcaccatgcagacggcatatctcctctggacccacacgatttctggagatgtcccgtaggggaacccctactgagcaacaaccccaatatctcattattacctggaccaagtctactttctggatccaccacaatttcaggatgtgttagactaccttcattatcaattggtgatgcaatatatgcgtattcatcaaacttaatcactcaaggatgtgcagatatagggaagtcatatcaggttttacaattaggttacatatccttaaattcagatatgtatcctgatttaaacccggtaatttctcatacctatgacatcaacgacaacaggaaatcatgttctgtaatagctgcaggaacaaggggttatcagttatgctccttgcccactgtgaatgagactacagactactcgagtgaaggtatagaagatttagtatttgacatattagatctcaagggaaagaccaaatctcatcgatacaaaaatgaagatataacttttgaccatcctttttctgcaatgtatccgagtgtaggaagtgggataaaaattgaaaatacactcattttcctagggtacggtggcttaacaactccgctccaaggcgacactaagtgtgtgataaacagatgtaccaatgttaatcagagtgtttgcaatgatgctcttaagataacttggctaaagaaaagacaagttgtcaatgtcttaattcgtatcaataattatttatctgataggccaaagattgttgtcgagacaattccaataactcaaaattacttaggtgccgaaggtaggctacttaaactaggtaaaaagatctacatatatactagatcttcaggttggcactccaacctgcaaataggatcattagatatcaacaaccccatgaccattaaatgggcgcctcatgaagtcctgtctcgaccaggaaaccaagactgcaactggtacaacagatgtccgagagaatgcatatcaggtgtatatactgatgcatatccactatctcctgatgcagtcaatgttgctacaaccacactgtacgcaaacacatcacgtgttaatcccaccataatgtactcaaatacctcagaaattatcaacatgctaagactcaagaatgtacaactagaggcagcatacactactacatcatgtatcactcatttcgggaagggctactgcttccacattgttgaaatcaaccaagccagccttaataccttacaacctatgttgttcaagacaagtatccctaaaatatgtaaaatcacatcttgagcagatcaagacccaacactatatcaattatgtgaaaaccagatatgatgtataaaaatttaaaaacaaagcatgaatagacatttatatgacaaatagaataagaaaaacttagggttaatgcctgcctatttgtcaaatatggataaacaggagtcaactcagaattcctcagacatcttatatccagaatgtcacttgaactctccgattgtaaaaagcaagattgctcaacttcacgttttgctagatatcaatcaaccctatgatttaaaagataacagtataataaatatcaccaaatacaaaatcagaaatggaggtttatcgccccggcagatcaaaatcagatcgctaggcaaaatccttaaacaagaaattaaggatattgatcgttacacttttgaaccttatccgattttctcattagagttactcagactggatatcccagaaatatgtgacaaaataagatccattttttcagtctctgatagattaataagagaactatcatctggatttcaagaattgtggttaaatattcttagacaattaggctgtgttgaagggaaagagggatttgactcattaaaggatgtagatatcatcccagatataactgataaatataataaaaacacatggtatcgcccattcttaacatggtttagcatcaaatatgatatgagatggatgcaaaagaataagtcggggaaccatttagatgtctcaaattctcacaattttcttgactgtaaatcatatattttgattatatatagagatttagtgataataataaataaattaaaattaaccggttatgtccttacacctgaattagtattaatgtattgtgatgttgtcgaaggaagatggaatatgtcttcagctggacgactcgataaaaggtcatcaaaaataacatgtaagggggaagaattatgggagcttatcgactctttatttcccaatcttggtgaggatgtatataatattatatcactactagaacctttatcacttgctttaatacagttggatgaccctgtaactaatttaaaaggagctttcatgagacatgttttgactgagctacatacaattttaataaaagataatatatacacagattcagaagcagacagcataatggaatcattgataaagattttcagagagacatcaattgatgaaaaagcagaaattttctccttttttagaacgtttggacatcctagcttagaagcaataactgctgccgataaagtaaggacacatatgtattcctccaaaaaaatcatactaaagacactatatgagtgtcatgcaatcttctgtgcaattataataaacggatatagagaaagacacggtggtcaatggccgccatgcgaattccccaatcatgtatgtcttgaactcaagaatgcacaaggatccaactctgcaatttcgtatgaatgtgccgtagacaattatagtagttttataggatttaaatttttaaaatttattgagcctcaattagatgaagatttgacaatttatatgaaggataaggctctatcacctaggaaagcagcatgggattcagtatatcccgacagtaatttatattacaaagtccctgaatcagaagagactcgtaggttaatcgaggtttttataaatgataataattttaaccctgcggatattattaattatgtagagtcaggagaatggttaaatgacgatagcttcaacatatcttacagtctcaaagaaaaagaaattaaacaagagggtcgactctttgccaagatgacatataagatgagagcagtccaggtattagcagaaacactactagcaaaaggagtaggtgagttattcagtgaaaatgggatggtaaagggagaaattgacctactaaagagactgactacattatctgtctcaggtgttccaagatccaactcagtttacaataatcccatattacatgagaaattgatcaaaaatatgaataagtgcaattcaaatgggtattgggatgaaagaaagaaatctaaaaatgaattcaaagctgcagactcatcaaccgaggggtatgagactctgagctgttttttaaccaccgatttgaaaaaatactgtctcaactggagatttgaaagtacagcgttgttcggtcaaagatgtaatgagatattcgggtttaaaactttctttaactggatgcaccctattctagaaaaaagtacaatttatgtaggagatccttactgtccagtacctgatagaatgcacaaagaactccaagatcatgatgataccggaatctttatccataatccaagagggggaatagagggttattgccagaaattatggacactaatctctattagtgcaatccatcttgcagctgttaaagttggtgtcagagtgtcagcaatggtacaaggagacaatcaagctatagcagtgacatccagagttcctgtcacacaaacctataagcaaaaaaagactcacgtctatgaagaaatcacaagatatttcggtgccttgagagaagttatgtttgatattggacatgaattaaaattaaatgagaccattataagtagcaaaatgtttgtatacagcaaacggatatattatgatgggaaaatcctcccacagtgcctcaaagctttaacaagatgtgtattttggtcagagactcttgtagatgaaaacaggtcagcatgctcaaacattgcaacatctatagccaaagctattgagaatggatattcacctatcttaggctattgtattgctctttttaaaacttgccaacaggtatgtatatcattaggaatgaccattaatcctactattacgtcaactatcaaagatcaatattttaaagggaaaaattggttaagatgtgcaatattgatcccagctaacataggagggttcaactatatgtctacagctagatgttttgtcagaaatataggtgatccagcagttgcagctctagcagacttaaagagattcatcaaagcaggtctgttagataaacaggtattatatcgtgtgatgaatcaagaaccaggagactcaagcttcttagattgggcatcagacccttattcatgcaatctcccacactcacaaagtataacaactataatcaaaaatgtaacagctagatcagtattgcaggaatcacctaatcctctcctatcaggtctcttttcagaatcaagtagtgaagaagatctcaacttagcatcatttttgatggataggaaagccatattgcccagagtagctcacgagatcttagataactcacttacaggtgtaagagaagctatagccgggatgcttgatacaacgaaatctctagtaagagctagtgtcaggagaggaggattatcatatagtatcttaagaagacttataaattatgatctattacaatatgagaccttaacaaggacactcagaaaaccggttaaggataatatagaatatgagtatatgtgttcagtagaattggcaataggattgaggcaaaaaatgtggtttcatctaacttatggaagaccaatccacggtttagaaactccagacccgttagaattattaagaggatcatttattgaaggctcagaaatatgtaaattttgtagatcagaagggaataaccctatgtatacttggttctatcttcctgacaacatcgacttagatacacttagcaatggaagtcctgccatacgtatcccttattttggttctgctactgatgaaagatcagaggctcaactaggttatgttaagaacttaagcaagccggcaaaagcagcaataagaatcgcaatggtttacacttgggcttatggaactgatgaaatatcatggatggaagcagcacttatagctcaaaccagggctaacttaagtttagagaatttgaagttactcacccctgtatcgacttctacaaatttgtcccacagattgagagatactgctacacagatgaaattttcaagtgctactttagttcgagcgagtcgatttattaccatatctaatgataatatggcattaaaagaggcaggagagtctaaagatactaatttagtttatcaacaaattatgttaaccggattgagcttatttgaattcaatatgaggtataaacaaggatcattatctaaacctatgatattacacttacatttgaataataaatgctgtatcatagaatctcctcaagaattgaatattcctcctagatctacattggacttagagatcactcaggaaaataacaagttaatctatgatcctgatcctctcaaggacatagatctagagttatttagtaaggttagggatgtagtacacacaattgatatgaattattggtctgatgatgaaataattagagcaactagtatatgtacagctatgactattgcagacacaatgtctcaattagatagagacaatcttaaagaaatgatagcactgataaatgatgatgatataaatagtttaatcaccgaatttatggttattgatatacccttattttgttccactttcgggggtattctaatcaatcaatttgcatattcactttacgggttaaacgtcagagggagggatgaaatatggggatatgtgatacgcataattaaagacacatcacatgcagtcctaaaagtactgtccaatgcattatcacatcctaaaatattcaaacgattctgggatgcaggagttgtagagcctgtttatggacctaacttgtccaatcaagacaagatactgttagccatttcagtatgtgaatactctgttgacctcttcatgcgtgattggcaagagggcataccgcttgaaatatttatttgtgataacgacccaaatatagcagaaatgagaaaactttcatttttagctagacatctagcatacttgtgtagtttggcagagatagctaaagagggaccaaaattggaatctatgacatctctcgaacgactcgaatcattgaaagagtatctagaacttacttttttagacgatcctatattaagatatagtcaattgacaggcttagttattaagatattcccttcaacgttaacttacatcaggaaatcttcaattaaggtgttgagagtaagaggtatagggataccagaagtcttagaggactgggatcctgatgccgatagtatgctactagataatataactgctgaggttcaacacaatatacctttaaagaagaacgaaagaactcccttctgggggttaagggtatcaaaatcacaagttctgcgacttagaggttatgaagagataaaaagggaagaaagaggaagatcaggtgtaggattaactctaccttttgatgggcgatatttatcacaccaattgagacttttcgggattaatagcaccagttgtttgaaagcattggaacttacctatttactgaatcctctagtcaataaggataaagatagattatatctcggagaaggtgcaggtgcaatgctgtcttgttatgatgctacattaggaccctgcatgaactattataattcaggtgttaattcttgtgatctcaacggacaaagagaattaaatatttatccttcagaagtggcactggtagggaagaaattgaataatgtcacgagtttatgtcaaagagttaaggttttattcaatgggaatcctggatcaacttggatagggaatgatgaatgtgaaacactaatctggaatgaattacagaataattcaatagggtttattcattgtgacatggaaggtggagaacacaaatgtgatcaggtggtcttacatgaacattatagtgtgatcaggattgcataccttgttggggataaggacgttatcttagtaagcaaaattgcaccaagattaggtacagactggacaaaacaattaagtttgtatttaagatactggagagatgtcagcttaatagtgttgaaaacatctaacccagcctctacagaaatgtatctgatatcaaaagatcctaaatctgatattatagaggatagtaatacagtattggcaaaccttcttccattatctaaagaggatagtattaagatagaaaaatggattctagttgagaaagccaaagttcatgattggatagttagagaattaaaggaagggagtgcatcgtcaggtatgctaagaccttaccatcaagcattacaaatcttcggatttgagcctaatttaaacaaattatgtagagatttcttatctacactaaatatagtagacacaaaaaattgtattatcacatttgatagagtattaagagatacaatctttgagtggactcggataaaagacgcagataagaagctaagacttacaggtaaatatgatctatatcctcttagagattcaggtaagttaaaagttatttctagaaggcttgtaatatcttggatagcattgtctatgtctacaagactagtaacagggtcatttccagacattaaatttgaatcaagactccaattaggtatagtatcaatatcctctcgtgaaatcaaaaatcttagggttatatcaaagattgtcattgacaaatttgaagatattatacatagtgtgacctataggttcttgactaaagaaataaaaatattgatgaaaattttgggagcagtcaaattatttggggcaagacagagcacatctgctgatatcactaatatcgatacatcggactccatacaatgatcttatatcttctcatctttattatctaatttgtttaaagagatgagttaacaagataagaaatccctttaactgactcataaaaacatagtaagaaaaacttacaacagacaagagtattaataatatatcgatatttcttaaactcttgtctggt

SEQ ID NO: 7 - DNA, 49 bp, Human parainfluenza virus
aagaatcactctcagatgtcatcggactccttgacgtcgtcttatccta

SEQ ID NO: 8 - DNA, 49 bp, Human parainfluenza virus
taggataagacgacgtcaaggagtccgatgacatctgagagtgattctt

SEQ ID NO: 9 - DNA, 49 bp, Artificial Sequence
  Description of Artificial Sequence Synthetic oligonucleotide primer derived from Human parainfluenza virus
aagaatcactctcagctgtcatcggactccttgtcgtcgtcttatccta

SEQ ID NO: 10 - DNA, 49 bp, Artificial Sequence
  Description of Artificial Sequence Synthetic oligonucleotide primer derived from Human parainfluenza virus
taggataagacgacgacaaggagtccgatgacagctgagagtgattctt

SEQ ID NO: 11 - DNA, 40 bp, Artificial Sequence
  Description of Artificial Sequence Synthetic oligonucleotide primer derived from Human parainfluenza virus
aacttagggtgaatgacaattcacagatcagctcaaccag

SEQ ID NO: 12 - DNA, 40 bp, Artificial Sequence
  Description of Artificial Sequence Synthetic oligonucleotide primer derived from Human parainfluenza virus
ttaattctttttgaatcccaatttcgggttgtacctagtc

SEQ ID NO: 13 - DNA, 39 bp, Human parainfluenza virus
ccttcccgagtggattaggatgcctagttttttgagagg

SEQ ID NO: 14 - DNA, 39 bp, Human parainfluenza virus
cctctcaaaaaactaggcatcctaatccactcgggaagg

SEQ ID NO: 15 - DNA, 39 bp, Artificial Sequence
  Description of Artificial Sequence Synthetic oligonucleotide primer derived from Human parainfluenza virus
ccttcccgagtggattaggctgcctagttttttgagagg

SEQ ID NO: 16 - DNA, 39 bp, Artificial Sequence
  Description of Artificial Sequence Synthetic oligonucleotide primer derived from Human parainfluenza virus
cctctcaaaaaactaggcagcctaatccactcgggaagg

SEQ ID NO: 17 - DNA, 40 bp, Artificial Sequence; identical to SEQ ID NO: 11
  Description of Artificial Sequence Synthetic oligonucleotide primer derived from Human parainfluenza virus

SEQ ID NO: 18 - DNA, 40 bp, Artificial Sequence; identical to SEQ ID NO: 12
  Description of Artificial Sequence Synthetic oligonucleotide primer derived from Human parainfluenza virus

SEQ ID NO: 19 - DNA, 96 bp, Human parainfluenza virus
ctcaaccagggcgcgccagcatacacgaaaccaaccttcacagtggatacctcagcatccaaaactctccttcccgagtggattaggatgcctagt

SEQ ID NO: 20 - DNA, 33 bp, Human parainfluenza virus
accaaagcacatacgaccgcgcgggaccaactc

SEQ ID NO: 21 - DNA, 15 bp, Human parainfluenza virus
tgatccgtaggatta

SEQ ID NO: 22 - DNA, 48 bp, Artificial Sequence
  Description of Artificial Sequence Synthetic oligonucleotide primer derived from Human parainfluenza virus
ctcaaccagggcgcgccagcatacacgaaaccaattaggatgcctagt

SEQ ID NO: 23 - DNA, 40 bp, Artificial Sequence; identical to SEQ ID NO: 12
  Description of Artificial Sequence Synthetic oligonucleotide primer derived from Human parainfluenza virus

SEQ ID NO: 24 - DNA, 54 bp, Human parainfluenza virus
acgaaaccaaccttcacagtggatacctcagcatccaaaactctccttcccgag

SEQ ID NO: 25 - DNA, 18 bp, Human parainfluenza virus
acacttccaaccaaagca

SEQ ID NO: 26 - DNA, 24 bp, Human parainfluenza virus
gagcccttcctctcaaaacctacg

SEQ ID NO: 27 - DNA, 42 bp, Artificial Sequence
  Description of Artificial Sequence Synthetic oligonucleotide primer derived from Human parainfluenza virus
acgaaaccaaccttcacagcatccaaaactctccttcccgag

SEQ ID NO: 28 - DNA, 42 bp, Artificial Sequence
  Description of Artificial Sequence Synthetic oligonucleotide primer derived from Human parainfluenza virus
tgctttggttggaagtgtcgtaggttttgagaggaagggctc

SEQ ID NO: 29 - DNA, 40 bp, Artificial Sequence; identical to SEQ ID NO: 11
  Description of Artificial Sequence Synthetic oligonucleotide primer derived from Human parainfluenza virus

SEQ ID NO: 30 - DNA, 40 bp, Artificial Sequence; identical to SEQ ID NO: 12
  Description of Artificial Sequence Synthetic oligonucleotide primer derived from Human parainfluenza virus

SEQ ID NO: 31 - DNA, 170 bp, Human parainfluenza virus
ggcgcgccagcatacacgaaaccaaccttcacagtggatacctcagcatccaaaactctccttcccgagtggattaggatgcctagttttttgagagggatcctgaagccgaaggagaggcaccacgaaaacaagaatcactctcagatgtcatcggactccttgacgtc

SEQ ID NO: 32 - DNA, 30 bp, Human parainfluenza virus
ctgcagttcctcaggctactgtagactctc

SEQ ID NO: 33 - DNA, 38 bp, Artificial Sequence
  Description of Artificial Sequence Synthetic oligonucleotide primer derived from Human parainfluenza virus
ggcgcgccctctcagatgtcatcggactccttgacgtc

SEQ ID NO: 34 - DNA, 38 bp, Artificial Sequence
  Description of Artificial Sequence Synthetic oligonucleotide primer derived from Human parainfluenza virus
gacgtcaaggagtccgatgacatctgagagggcgcgcc

SEQ ID NO: 35 - DNA, 36 bp, Artificial Sequence
  Description of Artificial Sequence Synthetic oligonucleotide primer derived from Human parainfluenza virus
gcgcgccctctcagatgtcatcggactccttgacgt

SEQ ID NO: 36 - DNA, 27 bp, Artificial Sequence
  Description of Artificial Sequence Synthetic oligonucleotide primer derived from Human parainfluenza virus
caaggagtccgatgacatctgagaggg

SEQ ID NO: 37 - DNA, 48 bp, Human parainfluenza virus
agggcgcgccctctcagatgtcatcggactccttgacgtcgtcttatc

SEQ ID NO: 38 - DNA, 48 bp, Human parainfluenza virus
gataagacgacgtcaaggagtccgatgacatctgagagggcgcgccct

SEQ ID NO: 39 - DNA, 48 bp, Artificial Sequence
  Description of Artificial Sequence Synthetic oligonucleotide primer derived from Human parainfluenza virus
agggcgcgccctctaagatgtcatcggactccttgtcgtcgtcttatc

SEQ ID NO: 40 - DNA, 48 bp, Artificial Sequence
  Description of Artificial Sequence Synthetic oligonucleotide primer derived from Human parainfluenza virus
gataagacgacgacaaggagtccgatgacatcttagagggcgcgccct

SEQ ID NO: 41 - DNA, 40 bp, Artificial Sequence; identical to SEQ ID NO: 11
  Description of Artificial Sequence Synthetic oligonucleotide primer derived from Human parainfluenza virus

SEQ ID NO: 42 - DNA, 40 bp, Artificial Sequence; identical to SEQ ID NO: 12
  Description of Artificial Sequence Synthetic oligonucleotide primer derived from Human parainfluenza virus

SEQ ID NO: 43 - DNA, 46 bp, Human parainfluenza virus
cgcgccctctcagatgtcatcggactccttgacgtcgtcttatcct

SEQ ID NO: 44 - DNA, 46 bp, Human parainfluenza virus
aggataagacgacgtcaaggagtccgatgacatctgagagggcgcg

SEQ ID NO: 45 - DNA, 46 bp, Artificial Sequence
  Description of Artificial Sequence Synthetic oligonucleotide primer derived from Human parainfluenza virus
cgcgccctctcagctgtcatcggactccgtgatgtcgtcttatcct

SEQ ID NO: 46 - DNA, 46 bp, Artificial Sequence
  Description of Artificial Sequence Synthetic oligonucleotide primer derived from Human parainfluenza virus
aggataagacgacatcacggagtccgatgacagctgagagggcgcg

SEQ ID NO: 47 - DNA, 40 bp, Artificial Sequence; identical to SEQ ID NO: 11
  Description of Artificial Sequence Synthetic oligonucleotide primer derived from Human parainfluenza virus

SEQ ID NO: 48 - DNA, 40 bp, Artificial Sequence; identical to SEQ ID NO: 12
  Description of Artificial Sequence Synthetic oligonucleotide primer derived from Human parainfluenza virus

SEQ ID NO: 49 - DNA, 46 bp, Human parainfluenza virus; identical to SEQ ID NO: 43

SEQ ID NO: 50 - DNA, 46 bp, Human parainfluenza virus; identical to SEQ ID NO: 44

SEQ ID NO: 51 - DNA, 46 bp, Artificial Sequence
  Description of Artificial Sequence Synthetic oligonucleotide primer derived from Human parainfluenza virus
cgcgccctctcagctgtcatcggactccgtgacgtcgtcttatcct

SEQ ID NO: 52 - DNA, 46 bp, Artificial Sequence
  Description of Artificial Sequence Synthetic oligonucleotide primer derived from Human parainfluenza virus
aggataagacgacgtcacggagtccgatgacagctgagagggcgcg

SEQ ID NO: 53 - DNA, 40 bp, Artificial Sequence; identical to SEQ ID NO: 11
  Description of Artificial Sequence Synthetic oligonucleotide primer derived from Human parainfluenza virus

SEQ ID NO: 54 - DNA, 40 bp, Artificial Sequence
  Description of Artificial Sequence Synthetic oligonucleotide primer derived from Human parainfluenza virus
ctgatccatgttgggctttaaccctaagtttttcttaatt

SEQ ID NO: 55 - DNA, 41 bp, Artificial Sequence
  Description of Artificial Sequence Synthetic oligonucleotide primer derived from Human parainfluenza virus
accttcacagtggataccccagcatccaaaactctccttcc

SEQ ID NO: 56 - DNA, 41 bp, Artificial Sequence
  Description of Artificial Sequence Synthetic oligonucleotide primer derived from Human parainfluenza virus
ggaaggagagttttggatgctggggtatccactgtgaaggt

SEQ ID NO: 57 - DNA, 25 bp, Artificial Sequence
  Description of Artificial Sequence Synthetic oligonucleotide primer derived from Human parainfluenza virus
ctgctgatcgggacaacaagaactg

SEQ ID NO: 58 - DNA, 40 bp, Artificial Sequence; identical to SEQ ID NO: 12
  Description of Artificial Sequence Synthetic oligonucleotide primer derived from Human parainfluenza virus